(19) United States
(12) Patent Application Publication
(10) Pub. No.: US 2026/0123962 A1
(43) Pub. Date: May 7, 2026

(54) HEAT TREATMENT OF A METAL ALLOY

(71) Applicant: MiRus LLC, Marietta, GA (US)

(72) Inventors: Noah Roth, Marietta, GA (US); Jay Yadav, Marietta, GA (US); Ravi Enneti, Marietta, GA (US); Jordan Bauman, Smyrna, GA (US)

(21) Appl. No.: 19/425,047

(22) Filed: Dec. 18, 2025

Related U.S. Application Data
(63) Continuation of application No. 18/222,237, filed on Jul. 14, 2023.
Application No. 18/222,237 is a continuation-in-part of application No. 17/586,270, filed on Jan. 27, 2022.
Application No. 18/222,237 is a continuation-in-part of application No. 18/116,677, filed on Mar. 2, 2023.
Application No. 18/222,237 is a continuation-in-part of application No. 17/876,282, filed on Jul. 28, 2022.
(60) Provisional application No. 63/226,270, filed on Jul. 28, 2021.
(60) Provisional application No. 63/247,540, filed on Sep. 23, 2021.
(60) Provisional application No. 63/316,077, filed on Mar. 3, 2022.
(60) Provisional application No. 63/389,267, filed on Jul. 14, 2022.

(51) Int. Cl. A61B 17/70; A61B 17/00; A61B 17/56; A61B 90/00; A61L 31/02; A61L 31/14; C22F 1/02

(52) U.S. Cl. CPC A61B 17/7031; A61B 17/7032; A61L 31/022; A61L 31/14; C22F 1/02; A61B 2017/00526; A61B 2017/564; A61B 2090/3937

(57) ABSTRACT

A medical device that includes special heat treated components and a method for heat treating the metal alloy for the medical device.

The present disclosure is a continuation of U.S. patent application Ser. No. 18/222,237 filed Jul. 14, 2023, which in turn is continuation in part of U.S. patent application Ser. No. 17/586,270 filed Jan. 27, 2022, which in turn claims priority on U.S. Provisional Application Ser. No. 63/226,270 filed Jul. 28, 2021, which is incorporated herein by reference.

The present disclosure is a continuation of U.S. patent application Ser. No. 18/222,237 filed Jul. 14, 2023, which in turn is a continuation in part of U.S. patent application Ser. No. 18/116,677 filed Mar. 2, 2023, which in turn claims priority on U.S. Provisional Application Ser. No. 63/316,077 filed Mar. 3, 2022, which is incorporated herein by reference.

The present disclosure is a continuation of U.S. patent application Ser. No. 18/222,237 filed Jul. 14, 2023, which in turn is a continuation in part of U.S. patent application Ser. No. 17/876,282 filed Jul. 28, 2022, which in turn claims priority on U.S. Provisional Application Ser. No. 63/247,540 filed Sep. 23, 2021, which is incorporated herein by reference.

The present disclosure is a continuation of U.S. patent application Ser. No. 18/222,237 filed Jul. 14, 2023, which in turn claims priority on U.S. Provisional Application Ser. No. 63/389,267 filed Jul. 14, 2022, which is incorporated herein by reference.

The disclosure relates generally to heat treatment of metals, more particularly to the heat treatment of refractory metal alloys or metal alloys that include at least 15 atomic weight percent (awt. %) rhenium, still more particularly to the heat treatment of refractory metal alloys or metal alloys that include at least 15 awt. % rhenium wherein the heat treated of the refractory metal alloys or the metal alloy that include at least 15 awt. % rhenium is to be used to partially or fully for a medical device, and yet more particularly to the heat treatment of refractory metal alloys or metal alloys that include at least 15 awt. % rhenium to produce a variable yield strength and/or ultimate tensile strength along the longitudinal length of the metal alloy and wherein the heat treated of the refractory metal alloy or metal alloy that include at least 15 awt. % rhenium is to be used to partially or fully for a medical device.

### BACKGROUND OF DISCLOSURE

Stainless steel, cobalt-chromium alloys, and TiAlV alloys are some of the more common metal alloys used for medical devices. Although these alloys have been successful in forming a variety of medical devices, these alloys have several deficiencies.

Refractory metal alloys and metal alloys that include at least 15 awt. % rhenium have been found to overcome many of these deficiencies. Refractory metal alloys and metal alloys that include at least 15 awt. % rhenium can be harder and stronger that other metal alloys. In some applications such as medical application for spinal surgery, the medical device such as a spinal rod that is formed of the metal alloy needs to have a certain degree of flexibility or bendability. To increase the flexibility or bendability of metal alloy rods, the rod is commonly ground to reduce the cross-sectional area of the rod to thereby make the rod more flexibly. As such, different sized rods are provided to surgeon during a spinal procedure. However, the issue with the use of rods of differing diameter is that different screws are commonly required for the different sized rods, which can complicate the surgery by having to keep track of the rods used in a medical procedure and can also potentially lead to the use of incorrect screws during a procedure.

### SUMMARY OF THE DISCLOSURE

The disclosure relates generally to heat treatment of metals, more particularly to the heat treatment of refractory metal alloys or metal alloys that include at least 15 atomic weight percent (awt. %) rhenium, still more particularly to the heat treatment of refractory metal alloys or metal alloys that include at least 15 awt. % rhenium wherein the heat treated of the refractory metal alloys or the metal alloy that include at least 15 awt. % rhenium is to be used to partially or fully for a medical device, and yet more particularly to the heat treatment of refractory metal alloys or metal alloys that include at least 15 awt. % rhenium to produce a variable yield strength and/or ultimate tensile strength along the longitudinal length of the metal alloy and wherein the heat treated of the refractory metal alloy or metal alloy that include at least 15 awt. % rhenium is to be used to partially or fully for a medical device. As defined herein, a refractory metal alloy is a metal alloy that includes at least 20 wt. % of one or more of molybdenum, rhenium, niobium, tantalum or tungsten. Non-limiting refractory metal alloys include MoRe alloy, ReW alloy, MoReCr alloy, MoReTa alloy, MoReTi alloy, WCu alloy, ReCr, molybdenum alloy, rhenium alloy, tungsten alloy, tantalum alloy, niobium alloy, etc.

In accordance with one non-limiting aspect of the present disclosure, the medical device can include, but is not limited to, an orthopedic device, PFO (patent foramen ovale) device, stent, valve (e.g., heart valve, TAVR valve, mitral valve replacement, tricuspid valve replacement, pulmonary valve replacement, etc.), spinal implant, spinal discs, frame and other structures for use with a spinal implant, vascular implant, graft, guide wire, sheath, catheter, needle, stent catheter, electrophysiology catheter, hypotube, staple, cutting device, any type of implant, pacemaker, dental implant, dental crown, dental braces, wire used in medical procedures, bone implant, artificial disk, artificial spinal disk, prosthetic implant or device to repair, replace and/or support a bone (e.g., acromion, atlas, axis, calcaneus, carpus, clavicle, coccyx, epicondyle, epitrochlea, femur, fibula, frontal bone, greater trochanter, humerus, ilium, ischium, mandible, maxilla, metacarpus, metatarsus, occipital bone, olecranon, parietal bone, patella, phalanx, radius, ribs, sacrum, scapula, sternum, talus, tarsus, temporal bone, tibia, ulna, zygomatic bone, etc.) and/or cartilage, bone plate, knee replacement, hip replacement, shoulder replacement, ankle replacement, nail, rod, screw, post, cage, plate, pedicle screw, cap, hinge, joint system, anchor, spacer, shaft, anchor, disk, ball, tension band, locking connector other structural assembly that is used in a body to support a structure, mount a structure, and/or repair a structure in a body such as, but not limited to, a human body, animal body, etc.

In accordance with one non-limiting aspect of the present disclosure, the metal alloy used to partially or fully form the medical device includes standard stainless steel, standard CoCr alloy, standard TiAlV alloy, standard aluminum alloy, standard nickel alloy, standard titanium alloy, standard tungsten alloy, standard molybdenum alloy, standard copper alloy, standard MP35N alloy, standard beryllium-copper alloy, refractory metal alloy, or a metal alloy that includes at least 15 atomic weight percent (awt. %) rhenium. As defined herein, a standard stainless-steel alloy includes 10-28 wt. % chromium, 0-35 wt. % nickel, 0-4 wt. % molybdenum, 0-2 wt. % manganese, 0-0.75 wt. % silicon, 0-0.3 wt. % carbon, 0-5 wt. % titanium, 0-10 wt. % niobium, 0-5 wt. % copper, 0-4 wt. % aluminum, 0-10 wt. % tantalum, 0-1 wt. % Se, 0-2 wt. % vanadium, 0-2 wt. % tungsten, and at least 50 wt. % iron. A standard 316L alloy includes 17-19 wt. % chromium, 13-15 wt. % nickel, 2-4 wt. % molybdenum, 2 wt. % max manganese, 0.75 wt. % max silicon, 0.03 wt. % max carbon, balance iron. As defined herein, a standard CoCr alloy includes 15-32 wt. % chromium, 1-36% wt. % nickel, 2-18 wt. % molybdenum, 0-18 wt. % iron, 0-1 wt. % titanium, 0-0.15 wt. % manganese, 0-0.15 wt. % silver, 0-0.025 wt. % carbon, 0-16 wt. % tungsten, 0-2 wt. % Si, 0-2 wt. % aluminum, 0-1 wt. % iron, 30-68 wt. % cobalt. As defined herein, a standard MP35N alloy includes 19-21 wt. % chromium, 34-36 wt. % nickel, 9-11 wt. % molybdenum, 1 wt. % max iron, 1 wt. % max titanium, 0.15 wt. % max manganese, 0.15 wt. % max silver, 0.025 wt. % max carbon, balance cobalt. As defined herein, a standard Phynox and standard Elgiloy alloy includes 38-42 wt. % cobalt, 18-22 wt. % chromium, 14-18 wt. % iron, 13-17 wt. % nickel, 6-8 wt. % molybdenum. As defined herein, a standard L605 alloy includes 18-22 wt. % chromium, 14-16 wt. % tungsten, 9-11 wt. % nickel, balance cobalt. As defined herein, a standard TiAlV alloy includes 5.5-6.75 wt. % aluminum, 3.5-4.5 wt. % vanadium, 85-93 wt. % titanium, 0-0.4 wt. % iron, 0-0.2 wt. % carbon. A standard Ti-6Al-4V alloy incudes 3.5-4.5 wt. % vanadium, 5.5-6.75 wt. % aluminum, 0.3 wt. % max iron, 0.2 wt. % max oxygen, 0.08 wt. % max carbon, 0.05 wt. % max nitrogen, 0.015 wt. % max hydrogen H, 0.05 wt. % max yttrium, balance titanium. As defined herein, a standard aluminum alloy includes 80-99 wt. % aluminum, 0-12 wt. % silicon, 0-5 wt. % magnesium, 0-1 wt. % manganese, 0-0.5 wt. % scandium, 0-0.5 wt. % beryllium, 0-0.5 wt. % yttrium, 0-0.5 wt. % cerium, 0-0.5 wt. % chromium, 0-3 wt. % iron, 0-0.5, 0-9 wt. % zinc, 0-0.5 wt. % titanium, 0-3 wt. % lithium, 0-0.5 wt. % silver, 0-0.5 wt. % calcium, 0-0.5 wt. % zirconium, 0-1 wt. % lead, 0-0.5 wt. % cadmium, 0-0.05 wt. % bismuth, 0-1 wt. % nickel, 0-0.2 wt. % vanadium, 0-0.1 wt. % gallium, and 0-7 wt. % copper. As defined herein, a standard nickel alloy includes 30-98 wt. % nickel, 5-25 wt. % chromium, 0-65 wt. % iron, 0-30 wt. % molybdenum, 0-32% wt. % copper, 0-32% wt. % cobalt, 2-2 wt. % aluminum, 0-6 wt. % tantalum, 0-15% wt. % tungsten, 0-5 wt. % titanium, 0-6 wt. % niobium, 0-3 wt. % silicon. As defined herein, a standard titanium alloy includes 80-99 wt. % titanium, 0-6 wt. % aluminum, 0-3 wt. % tin, 0-1 wt. % palladium, 0-8 wt. % vanadium, 0-15% wt. % molybdenum, 0-1 wt. % nickel, 0-0.3 wt. % ruthenium, 0-6 wt. % chromium, 0-4 wt. % zirconium, 0-4 wt. % niobium, 0-1 wt. % silicon, 0.0.5 wt. % cobalt, 0-2 wt. % iron. As defined herein, a standard tungsten alloy includes 85-98 wt. % tungsten, 0-8 wt. % nickel, 0-5 wt. % copper, 0-5 wt. % molybdenum, 0-4 wt. % iron. As defined herein, a standard molybdenum alloy includes 90-99.5 wt. % molybdenum, 0-1 wt. % nickel, 0-1 wt. % titanium, 0-1 wt. % zirconium, 0-30 wt. % tungsten, 0-2 wt. % hafnium, 0-2 wt. % lanthanum. As defined herein, a standard copper alloy includes 55-95 wt. % copper, 0-40 wt. % zinc, 0-10 wt. % tin, 0-10 wt. % lead, 0-1 wt. % iron, 0-5 wt. % silicon, 0-12 wt. % manganese, 0-12 wt. % aluminum, 0-3 wt. % beryllium, 0-1 Wt. % cobalt, 0-20% wt. % nickel. As defined herein, a standard MP35N alloy includes 32-38 wt. % nickel, 18-22 wt. % chromium, 8-12 wt. % molybdenum, 0-2 wt. % iron, 0-0.5 wt. % silicon, 0-0.5 wt. % manganese, 0-0.2 wt. % carbon, 0-2 wt. % titanium, 0-0.1 wt. % phosphorous, 0-0.1 wt. % boron, 0-0.1 wt. % sulfur, and the balance cobalt. As defined herein, a standard beryllium-copper alloy includes 95-98.5 wt. % copper, 1-4 wt. % beryllium, 0-1 wt. % cobalt, and 0-0.5 wt. % silicon. As defined herein, a refractory metal alloy is a metal alloy that includes at least 20 wt. % of one or more of molybdenum, rhenium, niobium, tantalum or tungsten. Non-limiting refractory metal alloys include MoRe alloy, ReW alloy, MoReCr alloy, MoReTa alloy, MoReTi alloy, WCu alloy, ReCr, molybdenum alloy, rhenium alloy, tungsten alloy, tantalum alloy, niobium alloy, etc.

In accordance with another and/or alternative aspect of the present disclosure, the metal alloy includes at least 15 awt. % rhenium, and at least 0.1 wt. % (e.g., 0.1 wt. % to 96 wt. % and all values and ranges therebetween) of one or more metals aluminum, bismuth, chromium, cobalt, copper, hafnium, iridium, iron, magnesium, manganese, molybdenum, nickel, niobium, osmium, rhodium, ruthenium, silicon, silver, tantalum, technetium, titanium, tungsten, vanadium, yttrium, zirconium, and which metal alloy exhibits a rhenium effect. As defined herein, a “rhenium effect” is a) an increase of at least 10% in ductility of the metal alloy caused by the addition of rhenium to the metal alloy, and/or b) an increase of at least 10% in tensile strength of the metal alloy caused by the addition of rhenium to the metal alloy. It has been found for many metal alloys (e.g., standard stainless steel, standard CoCr alloys, standard TiAlV alloys, standard aluminum alloys, standard nickel alloys, standard titanium alloys, standard tungsten alloys, standard molybdenum alloys, standard copper alloys, standard MP35N alloys, standard beryllium-copper alloys, etc.) results in improved ductility and/or tensile strength. It has been found that the addition of rhenium to a metal alloy can result in the formation of a twining alloy in the metal alloy that results in the overall ductility of the metal alloy to increase as the yield and tensile strength increases as a result of reduction and/or work hardening of the metal alloy that includes the rhenium addition. The rhenium effect occurs when the atomic weight of rhenium in the metal alloy is at least 15% (e.g., 15-99 awt. % rhenium in the metal alloy and all values and ranges therebetween). For example, for standard stainless-steel alloys, the rhenium effect can begin to be present when the stainless steel alloy is modified to include a rhenium amount of at least 5-10 wt. % (and all values and ranges therebetween) of the stainless steel alloy. For standard CoCr alloys, the rhenium effect can begin to be present when the CoCr alloy is modified to include a rhenium amount of at least 4.8-9.5 wt. % (and all values and ranges therebetween) of the CoCr alloy. For standard TiAlV alloys, the rhenium effect can begin to be present when the TiAlV alloy is modified to include a rhenium amount of at least 4.5-9 wt. % (and all values and ranges therebetween) of the TiAlV alloy. At can be appreciated, the rhenium content in the above examples can be greater than the minimum amount to create the rhenium effect in the metal alloy.

In accordance with another and/or alternative aspect of the present disclosure, the metal alloy includes at least 15 awt. % rhenium, and at least 0.1 wt. % (e.g., 0.1 wt. % to 96 wt. % and all values and ranges therebetween) of one or more metals aluminum, bismuth, chromium, cobalt, copper, hafnium, iridium, iron, magnesium, manganese, molybdenum, nickel, niobium, osmium, rhodium, ruthenium, silicon, silver, tantalum, technetium, titanium, tungsten, vanadium, yttrium, zirconium, and which metal alloy exhibits a rhenium effect.

In accordance with another and/or alternative aspect of the present disclosure, the metal alloy is a refractory metal alloy or a metal alloy that includes rhenium in a sufficient amount to create a rhenium effect in the metal alloy, and the metal alloy is a refractory metal alloy.

In accordance with another and/or alternative aspect of the present disclosure, the metal alloy is a stainless-steel alloy or a metal alloy that includes that includes rhenium in a sufficient amount to create a rhenium effect in the metal alloy, and the metal alloy is a standard stainless steel alloy that has been modified to include at least 15 awt. % rhenium.

In accordance with another and/or alternative aspect of the present disclosure, the metal alloy is a standard cobalt-chromium alloy or a metal alloy that includes that includes rhenium in a sufficient amount to create a rhenium effect in the metal alloy, and the metal alloy is a standard cobalt chromium alloy that has been modified to include at least 15 awt. % rhenium.

In accordance with another and/or alternative aspect of the present disclosure, the metal alloy is a standard TiAlV alloy or a metal alloy that includes that includes rhenium in a sufficient amount to create a rhenium effect in the metal alloy, and the metal alloy is a standard TiAlV alloy that has been modified to include at least 15 awt. % rhenium.

In accordance with another and/or alternative aspect of the present disclosure, the metal alloy is a standard aluminum alloy or a metal alloy that includes that includes rhenium in a sufficient amount to create a rhenium effect in the metal alloy, and the metal alloy is a standard aluminum alloy that has been modified to include at least 15 awt. % rhenium.

In accordance with another and/or alternative aspect of the present disclosure, the metal alloy is a standard nickel alloy or a metal alloy that includes that includes rhenium in a sufficient amount to create a rhenium effect in the metal alloy, and the metal alloy is a standard nickel alloy that has been modified to include at least 15 awt. % rhenium.

In accordance with another and/or alternative aspect of the present disclosure, the metal alloy is a standard titanium alloy or a metal alloy that includes that includes rhenium in a sufficient amount to create a rhenium effect in the metal alloy, and the metal alloy is a standard titanium alloy that has been modified to include at least 15 awt. % rhenium.

In accordance with another and/or alternative aspect of the present disclosure, the metal alloy is a standard tungsten alloy or a metal alloy that includes that includes rhenium in a sufficient amount to create a rhenium effect in the metal alloy, and the metal alloy is a standard tungsten alloy that has been modified to include at least 15 awt. % rhenium.

In accordance with another and/or alternative aspect of the present disclosure, the metal alloy is a standard molybdenum alloy or a metal alloy that includes that includes rhenium in a sufficient amount to create a rhenium effect in the metal alloy, and the metal alloy is a standard molybdenum alloy that has been modified to include at least 15 awt. % rhenium.

In accordance with another and/or alternative aspect of the present disclosure, the metal alloy is a standard copper alloy or a metal alloy that includes that includes rhenium in a sufficient amount to create a rhenium effect in the metal alloy, and the metal alloy is a standard copper alloy that has been modified to include at least 15 awt. % rhenium.

In accordance with another and/or alternative aspect of the present disclosure, the metal alloy is a standard MP35N alloy or a metal alloy that includes that includes rhenium in a sufficient amount to create a rhenium effect in the metal alloy, and the metal alloy is a standard MP35N alloy that has been modified to include at least 15 awt. % rhenium.

In accordance with another and/or alternative aspect of the present disclosure, the metal alloy is a standard beryllium-copper alloy alloy or a metal alloy that includes that includes rhenium in a sufficient amount to create a rhenium effect in the metal alloy, and the metal alloy is a standard beryllium-copper alloy that has been modified to include at least 15 awt. % rhenium.

Several non-limiting examples of metal alloys that can be used to partially or fully form the frame of the medical device are set forth below in weight percent:

| Component/Wt. % | Ex. 1 | Ex. 2 | Ex. 3 | Ex. 4 |
|---|---|---|---|---|
| Ag | 0-40% | 0-40% | 0-40% | 0-40% |
| Al | 0-95% | 0-95% | 0-40% | 0-40% |
| B | 0-3% | 0-2% | 0-1% | 0-1% |
| Be | 0-10% | 0-8% | 0-5% | 0-5% |
| Bi | 0-40% | 0-40% | 0-40% | 0-40% |
| Ca | 0-5% | 0-4% | 0-2% | 0-2% |
| Cd | 0-4% | 0-3% | 0-3% | 0-2% |
| Ce | 0-8% | 0-5% | 0-3% | 0-3% |
| Cr | 0-40% | 0-40% | 0-40% | 0-40% |
| Cu | 0-95% | 0-80% | 0-40% | 0-40% |
| Co | 0-70% | 0-70% | 0-70% | 0-60% |
| Fe | 0-90% | 0-80% | 0-80% | 0-80% |
| Ga | 0-3% | 0-2% | 0-1% | 0-1% |
| Hf | 0-40% | 0-40% | 0-40% | 0-40% |
| Ir | 0-40% | 0-40% | 0-40% | 0-40% |
| La | 0-5% | 0-5% | 0-4% | 0-3% |
| Li | 0-8% | 0-6% | 0-4% | 0-3% |
| Mg | 0-40% | 0-40% | 0-40% | 0-40% |
| Mn | 0-40% | 0-40% | 0-40% | 0-40% |
| Mo | 0-90% | 10-90% | 20-85% | 40-80% |
| Nb | 0-80% | 0-80% | 0-80% | 0-80% |
| Ni | 0-90% | 0-90% | 0-60% | 0-60% |
| Os | 0-40% | 0-40% | 0-40% | 0-40% |
| Pb | 0-5% | 0-4% | 0-45 | 0-2% |
| Pd | 0-4% | 0-3% | 0-2% | 0-2% |
| Pt | 0-40% | 0-40% | 0-40% | 0-40% |
| Re | 0-98% | 10-90% | 20-80% | 30-70% |
| Rh | 0-40% | 0-40% | 0-40% | 0-40% |
| Ru | 0-4% | 0-2% | 0-2% | 0-1% |
| Sc | 0-8% | 0-5% | 0-4% | 0-4% |
| Si | 0-40% | 0-40% | 0-40% | 0-40% |
| Sn | 0-40% | 0-40% | 0-40% | 0-40% |
| Ta | 0-80% | 0-60% | 0-80% | 0-80% |
| Tc | 0-40% | 0-40% | 0-40% | 0-40% |
| Ti | 0-90% | 0-80% | 0-60% | 0-60% |
| V | 0-40% | 0-40% | 0-40% | 0-40% |
| W | 0-94% | 0-94% | 0-90% | 0-90% |
| Y | 0-40% | 0-40% | 0-40% | 0-40% |
| Zr | 0-40% | 0-40% | 0-40% | 0-40% |
| Cs₂O | 0-1% | 0-1% | 0-1% | 0-1% |
| La₂O₃ | 0-3% | 0.1-2% | 0-2% | 0-2% |
| Y₂O₃ | 0-1% | 0-1% | 0.1-1% | 0-1% |
| ZrO₂ | 0-3% | 0-3% | 0-3% | 0-3% |
| C | <0.4% | <0.3% | <0.06% | <0.06% |
| N | <0.06% | <0.06% | <0.06% | <0.06% |
| O | <0.06% | <0.06% | <0.06% | <0.06% |
| P | <0.2% | <0.1% | <0.05% | <0.05% |
| S | <0.2% | <0.1% | <0.05% | <0.05% |
| H | <0.2% | <0.1% | <0.05% | <0.05% |
| Component/Wt. % | Ex. 5 | Ex. 6 | Ex. 7 | Ex. 8 |
| Ag | 0-20% | 0-20% | 0-20% | 0-20% |
| Al | 0-35% | 0-30% | 5-30% | 0-25% |
| Bi | 0-20% | 0-20% | 0-20% | 0-20% |
| Cr | 10-40% | 0-40% | 0-40% | 0-40% |
| Cu | 0-20% | 0-20% | 0-20% | 0-20% |
| Co | 10-60% | 0-60% | 0-60% | 0-60% |
| Fe | 0-80% | 30-80% | 0-80% | 0-70% |
| Hf | 0-20% | 0-20% | 0-20% | 0-20% |
| Ir | 0-20% | 0-20% | 0-20% | 0-20% |
| Mg | 0-20% | 0-20% | 0-20% | 0-20% |
| Mn | 0-20% | 0-40% | 0-20% | 0-20% |
| Mo | 0-60% | 0-60% | 0-80% | 0-70% |
| Nb | 0-60% | 0-60% | 0-65% | 20-60% |
| Ni | 0-60% | 5-55% | 0-52% | 0-50% |
| Os | 0-20% | 0-20% | 0-20% | 0-20% |
| Pt | 0-20% | 0-20% | 0-20% | 0-20% |
| Re | 4.5-98% | 4.5-90% | 4.5-80% | 4.5-70% |
| Rh | 0-20% | 0-20% | 0-20% | 0-20% |
| Si | 0-20% | 0-20% | 0-20% | 0-20% |
| Sn | 0-20% | 0-20% | 0-20% | 0-20% |
| Ta | 0-60% | 0-60% | 5-65% | 0-60% |
| Tc | 0-20% | 0-20% | 0-20% | 0-20% |
| Ti | 0-60% | 0-55% | 0-53% | 0-50% |
| V | 0-20% | 0-20% | 2-20% | 0-20% |
| W | 0-60% | 0-60% | 0-80% | 0-70% |
| Y | 0-20% | 0-20% | 0-20% | 0-20% |
| Zr | 0-20% | 0-20% | 0-20% | 5-20% |
| Cs₂O | 0-1% | 0-1% | 0-1% | 0-1% |
| La₂O₃ | 0-3% | 0.1-2% | 0-2% | 0-2% |
| Y₂O₃ | 0-1% | 0-1% | 0.1-1% | 0-1% |
| ZrO₂ | 0-3% | 0-3% | 0-3% | 0-3% |
| C | <0.06% | <0.06% | <0.06% | <0.06% |
| N | <0.06% | <0.06% | <0.06% | <0.06% |
| O | <0.06% | <0.06% | <0.06% | <0.06% |

| Component/Wt. % | Ex. 9 | Ex. 10 | Ex. 11 | Ex. 12 |
|---|---|---|---|---|
| Ag | 0-5% | 0-5% | 0-5% | 0-5% |
| Al | 0-5% | 0-5% | 1-15% | 0-20% |
| Bi | 0-5% | 0-5% | 0-5% | 0-5% |
| Cr | 1-28% | 1-30% | 0-5% | 0-30% |
| Cu | 0-20% | 0-5% | 0-5% | 0-25% |
| Co | 0-5% | 1-60% | 0-5% | 0-60% |
| Fe | 10-80% | 0-25% | 0-5% | 0-80% |
| Hf | 0-5% | 0-5% | 0-5% | 0-5% |
| Ir | 0-5% | 0-5% | 0-5% | 0-5% |
| Mg | 0-5% | 0-5% | 0-5% | 0-5% |
| Mn | 0-5% | 0-5% | 0-5% | 0-5% |
| Mo | 0-8% | 0-25% | 0-5% | 0-98% |
| Nb | 0-5% | 0-5% | 0-5% | 0-95% |
| Ni | 1-20% | 1-45% | 0-5% | 0-50% |
| Os | 0-5% | 0-5% | 0-5% | 0-5% |
| Pt | 0-5% | 0-5% | 0-5% | 0-5% |
| Re | 5-20% | 4.8-20% | 4.5-20% | 4.5-20% |
| Rh | 0-5% | 0-5% | 0-5% | 0-5% |
| Si | 0-5% | 0-5% | 0-5% | 0-5% |
| Sn | 0-5% | 0-5% | 0-5% | 0-5% |
| Ta | 0-5% | 0-5% | 0-5% | 0-98% |
| Tc | 0-5% | 0-5% | 0-5% | 0-5% |
| Ti | 0-5% | 0-5% | 40-93% | 0-93% |
| V | 0-5% | 0-5% | 1-10% | 0-20% |
| W | 0-5% | 0-20% | 0-5% | 0-98% |
| Y | 0-5% | 0-5% | 0-5% | 0-5% |
| Zr | 0-5% | 0-5% | 0-5% | 0-5% |
| Cs₂O | 0-1% | 0-1% | 0-1% | 0-1% |
| La₂O₃ | 0-3% | 0.1-2% | 0-2% | 0-2% |
| Y₂O₃ | 0-1% | 0-1% | 0.1-1% | 0-1% |
| ZrO₂ | 0-3% | 0-3% | 0-3% | 0-3% |
| C | <0.06% | <0.06% | <0.06% | <0.06% |
| N | <0.06% | <0.06% | <0.06% | <0.06% |
| O | <0.06% | <0.06% | <0.06% | <0.06% |

| Component/Wt. % | Ex. 13 | Ex. 14 | Ex. 15 | Ex. 16 |
|---|---|---|---|---|
| Mo | 40-80% | 40-80% | 40-80% | 40-80% |
| C | 0.01-0.3% | 0-0.3% | 0-0.3% | 0-0.3% |
| Co | ≤0.002% | ≤0.002% | ≤0.002% | ≤0.002% |
| Cs₂O | 0-0.2% | 0-0.2% | 0.01-0.2% | 0-0.2% |
| Fe | ≤0.02% | ≤0.02% | ≤0.02% | ≤0.02% |
| H | ≤0.002% | ≤0.002% | ≤0.002% | ≤0.002% |
| Hf | 0.1-2.5% | 0-2.5% | 0-2.5% | 0-2.5% |
| O | ≤0.06% | ≤0.06% | ≤0.06% | ≤0.06% |
| Os | ≤1% | ≤1% | ≤1% | ≤1% |
| La₂O₃ | 0-32% | 0.1-2% | 0-2% | 0-2% |
| N | ≤20 ppm | ≤20 ppm | ≤20 ppm | ≤20 ppm |
| Nb | ≤0.01% | ≤0.01% | ≤0.01% | ≤0.01% |
| Pt | ≤1% | ≤1% | ≤1% | ≤1% |
| Re | 7-49% | 7.5-49% | 7.5-49% | 7.5-49% |
| S | ≤0.008% | ≤0.008% | ≤0.008% | ≤0.008% |
| Sn | ≤0.002% | ≤0.002% | ≤0.002% | ≤0.002% |
| Ta | 0-50% | 0-50% | 0-50% | 0-50% |
| Tc | ≤1% | ≤1% | ≤1% | ≤1% |
| Ti | ≤1% | ≤1% | ≤1% | ≤1% |
| V | ≤1% | ≤1% | ≤1% | ≤1% |
| W | 0-50% | 0-50% | 0-50% | 0.5-50% |
| Y₂O₃ | 0-1% | 0-1% | 0.1-1% | 0-1% |
| Zr | ≤1% | ≤1% | ≤1% | ≤1% |
| ZrO₂ | 0-3% | 0-3% | 0-3% | 0-3% |
| CNT | 0-10% | 0-10% | 0-10% | 0-10% |
| C | <0.06% | <0.06% | <0.06% | <0.06% |
| N | <0.06% | <0.06% | <0.06% | <0.06% |
| O | <0.06% | <0.06% | <0.06% | <0.06% |

| Component/Wt. % | Ex. 17 | Ex. 18 | Ex. 19 |
|---|---|---|---|
| Mo | 40-80% | 40-80% | 40-80% |
| C | 0-0.3% | 0-0.3% | 0-0.3% |
| Co | ≤0.002% | ≤0.002% | ≤0.002% |
| Cs₂O | 0-0.2% | 0-0.2% | 0-0.2% |
| H | ≤0.002% | ≤0.002% | ≤0.002% |
| Hf | 0-2.5% | 0-2.5% | 0-2.5% |
| O | ≤0.06% | ≤0.06% | ≤0.06% |
| Os | ≤1% | ≤1% | ≤1% |
| La₂O₃ | 0-2% | 0-2% | 0-2% |
| N | ≤20 ppm | ≤20 ppm | ≤20 ppm |
| Nb | ≤0.01% | ≤0.01% | ≤0.01% |
| Pt | ≤1% | ≤1% | ≤1% |
| Re | 7-49% | 7.5-49% | 7.5-49% |
| S | ≤0.008% | ≤0.008% | ≤0.008% |
| Sn | ≤0.002% | ≤0.002% | ≤0.002% |
| Ta | 0-50% | 0.5-50% | 0-50% |
| Tc | ≤1% | ≤1% | ≤1% |
| Ti | ≤1% | ≤1% | ≤1% |
| V | ≤1% | ≤1% | ≤1% |
| W | 0-50% | 0-50% | 0-50% |
| Y₂O₃ | 0-1% | 0-1% | 0-1% |
| ZrO₂ | 0.1-3% | 0-3% | 0-3% |
| CNT | 0-10% | 0-10% | 0-10% |
| C | <0.06% | <0.06% | <0.06% |
| N | <0.06% | <0.06% | <0.06% |
| O | <0.06% | <0.06% | <0.06% |
| Component/Wt. % | Ex. 20 | Ex. 21 | Ex. 22 |
| Mo | 45-78% | 45-75% | 45-70% |
| C | 0-0.3% | 0-0.3% | 0-0.3% |
| Co | ≤0.002% | ≤0.002% | ≤0.002% |
| Cs₂O | 0-0.2% | 0-0.2% | 0-0.2% |
| H | ≤0.002% | ≤0.002% | ≤0.002% |
| Hf | 0-2.5% | 0-2.5% | 0-2.5% |
| O | ≤0.06% | ≤0.06% | ≤0.06% |
| Os | ≤1% | ≤1% | ≤1% |
| La₂O₃ | 0-2% | 0-2% | 0-2% |
| N | ≤20 ppm | ≤20 ppm | ≤20 ppm |
| Nb | ≤0.01% | ≤0.01% | ≤0.01% |
| Pt | ≤1% | ≤1% | ≤1% |
| Re | 7-49% | 7.5-49% | 7.5-49% |
| S | ≤0.008% | ≤0.008% | ≤0.008% |
| Sn | ≤0.002% | ≤0.002% | ≤0.002% |
| Ta | 0-50% | 0.5-50% | 0-50% |
| Tc | ≤1% | ≤1% | ≤1% |
| Ti | ≤1% | ≤1% | ≤1% |
| V | ≤1% | ≤1% | ≤1% |
| W | 0-50% | 0-50% | 0-50% |
| Y₂O₃ | 0-1% | 0-1% | 0-1% |
| ZrO₂ | 0.1-3% | 0-3% | 0-3% |
| CNT | 0-10% | 0-10% | 0-10% |
| C | <0.06% | <0.06% | <0.06% |
| N | <0.06% | <0.06% | <0.06% |
| O | <0.06% | <0.06% | <0.06% |

| Component/Wt. % | Ex. 23 | Ex. 24 | Ex. 25 | Ex. 26 |
|---|---|---|---|---|
| Mo | 35-80% | 35-80% | 35-70% | 35-65% |
| C | 0.05-0.15% | 0-0.15% | 0-0.15% | 0-0.15% |
| Cs₂O | 0-0.2% | 0-0.2% | 0.04-0.1% | 0-0.2% |
| Hf | 0.8-1.4% | 0-2% | 0-2.5% | 0-2.5% |
| La₂O₃ | 0-2% | 0.3-0.7% | 0-2% | 0-2% |
| Re | 7-49% | 7-49% | 7.5-49% | 7.5-49% |
| Ta | 0-2% | 0-2% | 0-50% | 0-50% |
| W | 0-2% | 0-2% | 0-50% | 20-50% |
| Y₂O₃ | 0-1% | 0-1% | 0.3-0.5% | 0-1% |
| ZrO₂ | 0-3% | 0-3% | 0-3% | 0-3% |

| Component/Wt. % | Ex. 27 | Ex. 28 | Ex. 29 |
|---|---|---|---|
| Mo | 40-60% | 35-60% | 30-60% |
| C | 0-0.15% | 0-0.15% | 0-0.15% |
| Cs₂O | 0-0.2% | 0-0.2% | 0-0.2% |
| Hf | 0-2.5% | 0-2.5% | 0-2.5% |
| La₂O₃ | 0-2% | 0-2% | 0-2% |
| Re | 7-60% | 7.5-65% | 7.5-70% |
| Ta | 0-3% | 10-50% | 0-40% |
| W | 0-3% | 0-50% | 0-40% |
| Y₂O₃ | 0-1% | 0-1% | 0-1% |
| ZrO₂ | 1.2-1.8% | 0-3% | 0-3% |

| Component/Wt. % | Ex. 30 | Ex. 31 | Ex. 32 |
|---|---|---|---|
| W | 20-80% | 60-80% | 20-78% |
| Re | 7.5-47.5% | 10-40% | 8-47.5% |
| Mo | 0-47.5% | <0.5% | 1-47.5% |
| Cu | <0.5% | <0.5% | <0.5% |
| C | ≤0.15% | ≤0.15% | ≤0.15% |
| Co | ≤0.002% | ≤0.002% | ≤0.002% |
| Cs₂O | ≤0.2% | ≤0.2% | ≤0.2% |
| Fe | ≤0.02% | ≤0.02% | ≤0.02% |
| H | ≤0.002% | ≤0.002% | ≤0.002% |
| Hf | <0.5% | <0.5% | <0.5% |
| La₂O₃ | <0.5% | <0.5% | <0.5% |
| O | ≤0.06% | ≤0.06% | ≤0.06% |
| Os | <0.5% | <0.5% | <0.5% |
| N | ≤20 ppm | ≤20 ppm | ≤20 ppm |
| Nb | ≤0.01% | ≤0.01% | ≤0.01% |
| Pt | <0.5% | <0.5% | <0.5% |
| S | ≤0.008% | ≤0.008% | ≤0.008% |
| Sn | ≤0.002% | ≤0.002% | ≤0.002% |
| Ta | <0.5% | <0.5% | <0.5% |
| Tc | <0.5% | <0.5% | <0.5% |
| Ti | <0.5% | <0.5% | <0.5% |
| V | <0.5% | <0.5% | <0.5% |
| Y₂O₃ | <0.5% | <0.5% | <0.5% |
| Zr | <0.5% | <0.5% | <0.5% |
| ZrO₂ | <0.5% | <0.5% | <0.5% |
| CNT | 0-10% | 0-10% | <0.5%. |
| C | <0.06% | <0.06% | <0.06% |
| N | <0.06% | <0.06% | <0.06% |
| O | <0.06% | <0.06% | <0.06% |

| Component/Wt. % | Ex. 33 | Ex. 34 | Ex. 35 |
|---|---|---|---|
| W | 20-80% | 60-80% | 20-75% |
| Re | 7.5-47.5% | 10-40% | 7.5-47.5% |
| Mo | 0-47.5% | <0.5% | 1-47.5% |
| Component/Wt. % | Ex. 36 | Ex. 37 | Ex. 38 |
| W | 50.1-80% | 65-80% | 50.1-79% |
| Re | 10-40% | 10-35% | 10-40% |
| Mo | 0-40% | <0.5% | 1-30% |
| Component/Wt. % | Ex. 39 | Ex. 40 | Ex. 41 |
| W | 20-49% | 20-49% | 20-49% |
| Re | 7.5-60% | 7.5-60% | 7.5-60% |
| Mo | 0-40% | 0-40% | 0-39% |
| Component/Wt. % | Ex. 42 | Ex. 43 | Ex. 44 |
| Re | 5-98% | 60-95% | 80-90% |
| Mo | 0-80% | 0-40% | 0-20% |
| W | 0-80% | 0-40% | 0-20% |
| Component/Wt. % | Ex. 45 | Ex. 46 | Ex. 47 |
| W | 20-49% | 20-49% | 20-49% |
| Re | 6-40% | 6-40% | 6-39% |
| Mo | 20-60% | 30-60% | 40-60% |
| Component/Wt. % | Ex. 48 | Ex. 49 | Ex. 50 |
| W | 20-40% | 20-35% | 20-30% |
| Re | 6-40% | 6-40% | 6-40% |
| Mo | 0-40% | 10-40% | 31-40% |

| Component/Wt. % | Ex. 51 | Ex. 52 | Ex. 53 | Ex. 54 |
|---|---|---|---|---|
| Re | 5-60% | 5-60% | 5-60% | 5-60% |
| Mo | 0-55% | 10-55% | 10-55% | 10-55% |
| Bi | 1-42% | 0-32% | 0-32% | 0-32% |
| Cr | 0-32% | 1-42% | 0-32% | 0-32% |
| Ir | 0-32% | 0-32% | 1-42% | 0-32% |
| Nb | 0-32% | 0-32% | 0-32% | 1-42% |
| Ta | 0-32% | 0-32% | 0-32% | 0-32% |
| Ti | 0-32% | 0-32% | 0-32% | 0-32% |
| Y | 0-32% | 0-32% | 0-32% | 0-32% |
| Zr | 0-32% | 0-32% | 0-32% | 0-32% |
| C | <0.06% | <0.06% | <0.06% | <0.06% |
| N | <0.06% | <0.06% | <0.06% | <0.06% |
| O | <0.06% | <0.06% | <0.06% | <0.06% |
| Component/Wt. % | Ex. 55 | Ex. 56 | Ex. 57 | Ex. 58 |
| Re | 5-60% | 5-60% | 5-60% | 5-60% |
| Mo | 15-55% | 15-55% | 15-55% | 15-55% |
| Bi | 0-32% | 0-32% | 0-32% | 0-32% |
| Cr | 0-32% | 0-32% | 0-32% | 0-32% |
| Ir | 0-32% | 0-32% | 0-32% | 0-32% |
| Nb | 0-32% | 0-32% | 0-32% | 0-32% |
| Ta | 1-42% | 0-32% | 0-32% | 0-32% |
| Ti | 0-32% | 1-42% | 0-32% | 0-32% |
| Y | 0-32% | 0-32% | 1-42% | 0-32% |
| Zr | 0-32% | 0-32% | 0-32% | 1-42% |
| C | <0.06% | <0.06% | <0.06% | <0.06% |
| N | <0.06% | <0.06% | <0.06% | <0.06% |
| O | <0.06% | <0.06% | <0.06% | <0.06% |
| Component/Wt. % | Ex. 59 | Ex. 60 | Ex. 61 | Ex. 62 |
| Re | 41-59% | 41-59% | 41-59% | 41-59% |
| Mo | 18-45% | 18-45% | 18-45% | 18-45% |
| Bi | 1-42% | 0-32% | 0-32% | 0-32% |
| Cr | 0-32% | 1-42% | 0-32% | 0-32% |
| Ir | 0-32% | 0-32% | 1-42% | 0-32% |
| Nb | 0-32% | 0-32% | 0-32% | 1-42% |
| Ta | 0-32% | 0-32% | 0-32% | 0-32% |
| Ti | 0-32% | 0-32% | 0-32% | 0-32% |
| Y | 0-32% | 0-32% | 0-32% | 0-32% |
| Zr | 0-32% | 0-32% | 0-32% | 0-32% |
| C | <0.06% | <0.06% | <0.06% | <0.06% |
| N | <0.06% | <0.06% | <0.06% | <0.06% |
| O | <0.06% | <0.06% | <0.06% | <0.06% |
| Component/Wt. % | Ex. 63 | Ex. 64 | Ex. 65 | Ex. 66 |
| Re | 41-59% | 41-59% | 41-59% | 41-59% |
| Mo | 18-45% | 18-45% | 18-45% | 18-45% |
| Bi | 0-32% | 0-32% | 0-32% | 0-32% |
| Cr | 0-32% | 0-32% | 0-32% | 0-32% |
| Ir | 0-32% | 0-32% | 0-32% | 0-32% |
| Nb | 0-32% | 0-32% | 0-32% | 0-32% |
| Ta | 1-42% | 0-32% | 0-32% | 0-32% |
| Ti | 0-32% | 1-42% | 0-32% | 0-32% |
| Y | 0-32% | 0-32% | 1-42% | 0-32% |
| Zr | 0-32% | 0-32% | 0-32% | 1-42% |
| C | <0.06% | <0.06% | <0.06% | <0.06% |
| N | <0.06% | <0.06% | <0.06% | <0.06% |
| O | <0.06% | <0.06% | <0.06% | <0.06% |
| Component/Wt. % | Ex. 67 | Ex. 68 | Ex. 69 | Ex. 70 |
| Re | 41-59% | 41-59% | 41-59% | 41-59% |
| Mo | 18-45% | 18-45% | 18-45% | 18-45% |
| Bi | 0-15% | 0-15% | 1-36% | 0-15% |
| Cr | 1-20% | 1-20% | 1-20% | 1-20% |
| Ir | 0-15% | 0-15% | 0-15% | 0-15% |
| Nb | 1-36% | 0-15% | 0-15% | 0-15% |
| Ta | 0-15% | 1-36% | 0-15% | 0-15% |
| Ti | 0-15% | 0-15% | 0-15% | 0-15% |
| Y | 0-15% | 0-15% | 0-15% | 0-15% |
| Zr | 0-15% | 0-15% | 0-15% | 1-36% |
| C | <0.06% | <0.06% | <0.06% | <0.06% |
| N | <0.06% | <0.06% | <0.06% | <0.06% |
| O | <0.06% | <0.06% | <0.06% | <0.06% |
| Component/Wt. % | Ex. 71 | Ex. 72 | Ex. 73 | Ex. 74 |
| Re | 41-59% | 41-59% | 41-59% | 41-59% |
| Mo | 18-45% | 18-45% | 18-45% | 18-45% |
| Bi | 1-36% | 0-15% | 0-15% | 0-15% |
| Cr | 1-20% | 1-20% | 1-20% | 1-20% |
| Ir | 0-15% | 1-36% | 0-15% | 0-15% |
| Nb | 0-15% | 0-15% | 0-15% | 0-15% |
| Ta | 0-15% | 0-15% | 0-15% | 0-15% |
| Ti | 0-15% | 0-15% | 1-36% | 0-15% |
| Y | 0-15% | 0-15% | 0-15% | 1-36% |
| Zr | 0-15% | 0-15% | 0-15% | 0-15% |
| C | <0.06% | <0.06% | <0.06% | <0.06% |
| N | <0.06% | <0.06% | <0.06% | <0.06% |
| O | <0.06% | <0.06% | <0.06% | <0.06% |
| Component/Wt. % | Ex. 75 | Ex. 76 | Ex. 77 | Ex. 78 |
| Re | 41-59% | 41-59% | 41-59% | 41-59% |
| Mo | 18-45% | 18-45% | 18-45% | 18-45% |
| Bi | 1-34% | 0-15% | 0-15% | 0-15% |
| Cr | 0-15% | 0-15% | 0-15% | 0-15% |
| Ir | 0-15% | 0-15% | 0-15% | 1-34% |
| Nb | 3-27% | 3-27% | 3-27% | 3-27% |
| Ta | 0-42 | 1-34% | 0-15% | 0-15% |
| Ti | 0-15% | 0-15% | 0-15% | 0-15% |
| Y | 0-15% | 0-15% | 0-15% | 0-15% |
| Zr | 0-15% | 0-15% | 3-27% | 0-15% |
| C | <0.06% | <0.06% | <0.06% | <0.06% |
| N | <0.06% | <0.06% | <0.06% | <0.06% |
| O | <0.06% | <0.06% | <0.06% | <0.06% |
| Component/Wt. % | Ex. 79 | Ex. 80 | Ex. 81 | Ex. 82 |
| Re | 41-59% | 41-59% | 41-59% | 41-59% |
| Mo | 18-45% | 18-45% | 18-45% | 18-45% |
| Bi | 0-15% | 0-15% | 0-15% | 0-15% |
| Cr | 0-15% | 0-15% | 0-15% | 0-15% |
| Ir | 0-15% | 1-34% | 0-15% | 0-15% |
| Nb | 0-15% | 0-15% | 0-15% | 0-15% |
| Ta | 1-34% | 0-15% | 3-27% | 0-15% |
| Ti | 0-15% | 0-15% | 0-15% | 0-15% |
| Y | 0-15% | 0-15% | 0-15% | 3-27% |
| Zr | 3-27% | 3-27% | 3-27% | 3-27% |
| C | <0.06% | <0.06% | <0.06% | <0.06% |
| N | <0.06% | <0.06% | <0.06% | <0.06% |
| O | <0.06% | <0.06% | <0.06% | <0.06% |
| Component/Wt. % | Ex. 83 | Ex. 84 | Ex. 85 | Ex. 86 |
| Re | 41-59% | 41-59% | 41-59% | 41-59% |
| Mo | 18-45% | 18-45% | 18-45% | 18-45% |
| Bi | 0-15% | 0-15% | 0-15% | 0-15% |
| Cr | 0-15% | 0-15% | 0-15% | 1-10% |
| Ir | 1-34% | 0-25 | 3-27% | 0-15% |
| Nb | 0-15% | 3-27% | 0-15% | 0-15% |
| Ta | 0-15% | 0-15% | 1-34% | 0-15% |
| Ti | 0-15% | 0-15% | 0-15% | 0-15% |
| Y | 3-27% | 3-27% | 0-15% | 0-15% |
| Zr | 0-15% | 0-15% | 3-27% | 1-12% |
| C | <0.06% | <0.06% | <0.06% | <0.06% |
| N | <0.06% | <0.06% | <0.06% | <0.06% |
| O | <0.06% | <0.06% | <0.06% | <0.06% |
| Component/Wt. % | Ex. 87 | Ex. 88 | Ex. 89 | Ex. 90 |
| Re | 50-75% | 55-75% | 60-75% | 65-75% |
| Cr | 25-50% | 25-45% | 25-40% | 25-35% |
| Mo | 0-25% | 0-25% | 0-25% | 0-25% |
| Bi | 0-25% | 0-25% | 0-25% | 0-25% |
| Cr | 0-25% | 0-25% | 0-25% | 0-25% |
| Ir | 0-25% | 0-25% | 0-25% | 0-25% |
| Nb | 0-25% | 0-25% | 0-25% | 0-25% |
| Ta | 0-25% | 0-25% | 0-25% | 0-25% |
| V | 0-25% | 0-25% | 0-25% | 0-25% |
| W | 0-25% | 0-25% | 0-25% | 0-25% |
| Mn | 0-25% | 0-25% | 0-25% | 0-25% |
| Tc | 0-25% | 0-25% | 0-25% | 0-25% |
| Ru | 0-25% | 0-25% | 0-25% | 0-25% |
| Rh | 0-25% | 0-25% | 0-25% | 0-25% |
| Hf | 0-25% | 0-25% | 0-25% | 0-25% |
| Os | 0-25% | 0-25% | 0-25% | 0-25% |
| Cu | 0-25% | 0-25% | 0-25% | 0-25% |
| Ir | 0-25% | 0-25% | 0-25% | 0-25% |
| Ti | 0-25% | 0-25% | 0-25% | 0-25% |
| Y | 0-25% | 0-25% | 0-25% | 0-25% |
| Zr | 0-25% | 0-25% | 0-25% | 0-25% |
| Ag | 0-25% | 0-25% | 0-25% | 0-25% |
| Al | 0-25% | 0-25% | 0-25% | 0-22% |
| Co | 0-25% | 0-25% | 0-25% | 0-25% |
| Fe | 0-25% | 0-25% | 0-25% | 0-25% |
| Mg | 0-25% | 0-25% | 0-25% | 0-25% |
| Ni | 0-25% | 0-25% | 0-25% | 0-25% |
| Pt | 0-25% | 0-25% | 0-25% | 0-25% |
| Si | 0-25% | 0-25% | 0-25% | 0-25% |
| Sn | 0-25% | 0-25% | 0-25% | 0-25% |
| Cs₂O | 0-1% | 0-1% | 0-1% | 0-1% |
| La₂O₃ | 0-3% | 0.1-2% | 0-2% | 0-2% |
| Y₂O₃ | 0-1% | 0-1% | 0.1-1% | 0-1% |
| ZrO₂ | 0-3% | 0-3% | 0-3% | 0-3% |
| C | <0.06% | <0.06% | <0.06% | <0.06% |
| N | <0.06% | <0.06% | <0.06% | <0.06% |
| O | <0.06% | <0.06% | <0.06% | <0.06% |
| Component/Wt. % | Ex. 91 | Ex. 92 | Ex. 93 | Ex. 94 |
| Re | 50-72% | 55-72% | 60-72% | 65-72% |
| Cr | 28-50% | 28-45% | 28-40% | 28-35% |
| Mo | 0-25% | 0-25% | 0-25% | 0-25% |
| Bi | 0-10% | 0-10% | 0-10% | 0-10% |
| Cr | 0-10% | 0-10% | 0-10% | 0-10% |
| Ir | 0-10% | 0-10% | 0-10% | 0-10% |
| Nb | 0-10% | 0-10% | 0-10% | 0-10% |
| Ta | 0-10% | 0-10% | 0-10% | 0-10% |
| V | 0-10% | 0-10% | 0-10% | 0-10% |
| W | 0-10% | 0-10% | 0-10% | 0-10% |
| Mn | 0-10% | 0-10% | 0-10% | 0-10% |
| Tc | 0-10% | 0-10% | 0-10% | 0-10% |
| Ru | 0-10% | 0-10% | 0-10% | 0-10% |
| Rh | 0-10% | 0-10% | 0-10% | 0-10% |
| Hf | 0-10% | 0-10% | 0-10% | 0-10% |
| Os | 0-10% | 0-10% | 0-10% | 0-10% |
| Cu | 0-10% | 0-10% | 0-10% | 0-10% |
| Ir | 0-10% | 0-10% | 0-10% | 0-10% |
| Ti | 0-10% | 0-10% | 0-10% | 0-10% |
| Y | 0-10% | 0-10% | 0-10% | 0-10% |
| Zr | 0-10% | 0-10% | 0-10% | 0-10% |
| Ag | 0-10% | 0-10% | 0-10% | 0-10% |
| Al | 0-10% | 0-10% | 0-10% | 0-10% |
| Co | 0-10% | 0-10% | 0-10% | 0-10% |
| Fe | 0-10% | 0-10% | 0-10% | 0-10% |
| Mg | 0-10% | 0-10% | 0-10% | 0-10% |
| Ni | 0-10% | 0-10% | 0-10% | 0-10% |
| Pt | 0-10% | 0-10% | 0-10% | 0-10% |
| Si | 0-10% | 0-10% | 0-10% | 0-10% |
| Sn | 0-10% | 0-10% | 0-10% | 0-10% |
| Cs₂O | 0-1% | 0-1% | 0-1% | 0-1% |
| La₂O₃ | 0-3% | 0.1-2% | 0-2% | 0-2% |
| Y₂O₃ | 0-1% | 0-1% | 0.1-1% | 0-1% |
| ZrO₂ | 0-3% | 0-3% | 0-3% | 0-3% |
| C | <0.06% | <0.06% | <0.06% | <0.06% |
| N | <0.06% | <0.06% | <0.06% | <0.06% |
| O | <0.06% | <0.06% | <0.06% | <0.06% |
| Component/Wt. % | Ex. 95 | Ex. 96 | Ex. 97 | Ex. 98 |
| Re | 50-70% | 55-70% | 60-70% | 65-70% |
| Cr | 30-50% | 30-45% | 30-40% | 30-35% |
| Mo | 0-10% | 0-10% | 0-10% | 0-10% |
| Bi | 0-10% | 0-10% | 0-10% | 0-10% |
| Cr | 0-10% | 0-10% | 0-10% | 0-10% |
| Ir | 0-10% | 0-10% | 0-10% | 0-10% |
| Nb | 0-10% | 0-10% | 0-10% | 0-10% |
| Ta | 0-10% | 0-10% | 0-10% | 0-10% |
| V | 0-10% | 0-10% | 0-10% | 0-10% |
| W | 0-10% | 0-10% | 0-10% | 0-10% |
| Mn | 0-10% | 0-10% | 0-10% | 0-10% |
| Tc | 0-10% | 0-10% | 0-10% | 0-10% |
| Ru | 0-10% | 0-10% | 0-10% | 0-10% |
| Rh | 0-10% | 0-10% | 0-10% | 0-10% |
| Hf | 0-10% | 0-10% | 0-10% | 0-10% |
| Os | 0-10% | 0-10% | 0-10% | 0-10% |
| Cu | 0-10% | 0-10% | 0-10% | 0-10% |
| Ir | 0-10% | 0-10% | 0-10% | 0-10% |
| Ti | 0-10% | 0-10% | 0-10% | 0-10% |
| Y | 0-10% | 0-10% | 0-10% | 0-10% |
| Zr | 0-10% | 0-10% | 0-10% | 0-10% |
| Ag | 0-10% | 0-10% | 0-10% | 0-10% |
| Al | 0-10% | 0-10% | 0-10% | 0-10% |
| Co | 0-10% | 0-10% | 0-10% | 0-10% |
| Fe | 0-10% | 0-10% | 0-10% | 0-10% |
| Mg | 0-10% | 0-10% | 0-10% | 0-10% |
| Ni | 0-10% | 0-10% | 0-10% | 0-10% |
| Pt | 0-10% | 0-10% | 0-10% | 0-10% |
| Si | 0-10% | 0-10% | 0-10% | 0-10% |
| Sn | 0-10% | 0-10% | 0-10% | 0-10% |
| Cs₂O | 0-1% | 0-1% | 0-1% | 0-1% |
| La₂O₃ | 0-3% | 0.1-2% | 0-2% | 0-2% |
| Y₂O₃ | 0-1% | 0-1% | 0.1-1% | 0-1% |
| ZrO₂ | 0-3% | 0-3% | 0-3% | 0-3% |
| C | <0.06% | <0.06% | <0.06% | <0.06% |
| N | <0.06% | <0.06% | <0.06% | <0.06% |
| O | <0.06% | <0.06% | <0.06% | <0.06% |

| Component/Wt. % | Ex. 99 | Ex. 100 | Ex. 101 | Ex. 102 |
|---|---|---|---|---|
| Re | 50-67.5% | 55-67.5% | 60-67.5% | 65-67.5% |
| Cr | 32.5-50% | 32.5-45% | 32.5-40% | 32.5-35% |
| Mo | 0-10% | 0-10% | 0-10% | 0-10% |
| Bi | 0-10% | 0-10% | 0-10% | 0-10% |
| Cr | 0-10% | 0-10% | 0-10% | 0-10% |
| Ir | 0-10% | 0-10% | 0-10% | 0-10% |
| Nb | 0-10% | 0-10% | 0-10% | 0-10% |
| Ta | 0-10% | 0-10% | 0-10% | 0-10% |
| V | 0-10% | 0-10% | 0-10% | 0-10% |
| W | 0-10% | 0-10% | 0-10% | 0-10% |
| Mn | 0-10% | 0-10% | 0-10% | 0-10% |
| Tc | 0-10% | 0-10% | 0-10% | 0-10% |
| Ru | 0-10% | 0-10% | 0-10% | 0-10% |
| Rh | 0-10% | 0-10% | 0-10% | 0-10% |
| Hf | 0-10% | 0-10% | 0-10% | 0-10% |
| Os | 0-10% | 0-10% | 0-10% | 0-10% |
| Cu | 0-10% | 0-10% | 0-10% | 0-10% |
| Ir | 0-10% | 0-10% | 0-10% | 0-10% |
| Ti | 0-10% | 0-10% | 0-10% | 0-10% |
| Y | 0-10% | 0-10% | 0-10% | 0-10% |
| Zr | 0-10% | 0-10% | 0-10% | 0-10% |
| Ag | 0-10% | 0-10% | 0-10% | 0-10% |
| Al | 0-10% | 0-10% | 0-10% | 0-10% |
| Co | 0-10% | 0-10% | 0-10% | 0-10% |
| Fe | 0-10% | 0-10% | 0-10% | 0-10% |
| Mg | 0-10% | 0-10% | 0-10% | 0-10% |
| Ni | 0-10% | 0-10% | 0-10% | 0-10% |
| Pt | 0-10% | 0-10% | 0-10% | 0-10% |
| Si | 0-10% | 0-10% | 0-10% | 0-10% |
| Sn | 0-10% | 0-10% | 0-10% | 0-10% |
| Cs₂O | 0-1% | 0-1% | 0-1% | 0-1% |
| La₂O₃ | 0-3% | 0.1-2% | 0-2% | 0-2% |
| Y₂O₃ | 0-1% | 0-1% | 0.1-1% | 0-1% |
| ZrO₂ | 0-3% | 0-3% | 0-3% | 0-3% |
| C | <0.06% | <0.06% | <0.06% | <0.06% |
| N | <0.06% | <0.06% | <0.06% | <0.06% |
| O | <0.06% | <0.06% | <0.06% | <0.06% |

| Component/Wt. % | Ex. 103 | Ex. 104 | Ex. 105 | Ex. 106 |
|---|---|---|---|---|
| Re | 50-67.5% | 55-67.5% | 60-67.5% | 65-67.5% |
| Cr | 32.5-50% | 32.5-45% | 32.5-40% | 32.5-35% |
| Mo | 0-5% | 0-5% | 0-5% | 0-5% |
| Bi | 0-5% | 0-5% | 0-5% | 0-5% |
| Cr | 0-5% | 0-5% | 0-5% | 0-5% |
| Ir | 0-5% | 0-5% | 0-5% | 0-5% |
| Nb | 0-5% | 0-5% | 0-5% | 0-5% |
| Ta | 0-5% | 0-5% | 0-5% | 0-5% |
| V | 0-5% | 0-5% | 0-5% | 0-5% |
| W | 0-5% | 0-5% | 0-5% | 0-5% |
| Mn | 0-5% | 0-5% | 0-5% | 0-5% |
| Tc | 0-5% | 0-5% | 0-5% | 0-5% |
| Ru | 0-5% | 0-5% | 0-5% | 0-5% |
| Rh | 0-5% | 0-5% | 0-5% | 0-5% |
| Hf | 0-5% | 0-5% | 0-5% | 0-5% |
| Os | 0-5% | 0-5% | 0-5% | 0-5% |
| Cu | 0-5% | 0-5% | 0-5% | 0-5% |
| Ir | 0-5% | 0-5% | 0-5% | 0-5% |
| Ti | 0-5% | 0-5% | 0-5% | 0-5% |
| Y | 0-5% | 0-5% | 0-5% | 0-5% |
| Zr | 0-5% | 0-5% | 0-5% | 0-5% |
| Ag | 0-5% | 0-5% | 0-5% | 0-5% |
| Al | 0-5% | 0-5% | 0-5% | 0-5% |
| Co | 0-5% | 0-5% | 0-5% | 0-5% |
| Fe | 0-5% | 0-5% | 0-5% | 0-5% |
| Mg | 0-5% | 0-5% | 0-5% | 0-5% |
| Ni | 0-5% | 0-5% | 0-5% | 0-5% |
| Pt | 0-5% | 0-5% | 0-5% | 0-5% |
| Si | 0-5% | 0-5% | 0-5% | 0-5% |
| Sn | 0-5% | 0-5% | 0-5% | 0-5% |
| Cs₂O | 0-1% | 0-1% | 0-1% | 0-1% |
| La₂O₃ | 0-3% | 0.1-2% | 0-2% | 0-2% |
| Y₂O₃ | 0-1% | 0-1% | 0.1-1% | 0-1% |
| ZrO₂ | 0-3% | 0-3% | 0-3% | 0-3% |
| C | <0.06% | <0.06% | <0.06% | <0.06% |
| N | <0.06% | <0.06% | <0.06% | <0.06% |
| O | <0.06% | <0.06% | <0.06% | <0.06% |

| Component/Wt. % | Ex. 107 | Ex. 108 | Ex. 109 | Ex. 110 |
|---|---|---|---|---|
| Re | 50-75% | 55-72% | 60-70% | 62-70% |
| Cr | 24-49% | 27-44% | 29-39% | 29-37% |
| Mo | 1-15% | 1-10% | 1-8% | 1-5% |
| Bi | 0-15% | 0-10% | 0-8% | 0-5% |
| Ir | 0-15% | 0-10% | 0-8% | 0-5% |
| Nb | 0-15% | 0-10% | 0-8% | 0-5% |
| Ta | 0-15% | 0-10% | 0-8% | 0-5% |
| V | 0-15% | 0-10% | 0-8% | 0-5% |
| W | 0-15% | 0-10% | 0-8% | 0-5% |
| Mn | 0-15% | 0-10% | 0-8% | 0-5% |
| Tc | 0-15% | 0-10% | 0-8% | 0-5% |
| Ru | 0-15% | 0-10% | 0-8% | 0-5% |
| Rh | 0-15% | 0-10% | 0-8% | 0-5% |
| Hf | 0-15% | 0-10% | 0-8% | 0-5% |
| Os | 0-15% | 0-10% | 0-8% | 0-5% |
| Cu | 0-15% | 0-10% | 0-8% | 0-5% |
| Ir | 0-15% | 0-10% | 0-8% | 0-5% |
| Ti | 0-15% | 0-10% | 0-8% | 0-5% |
| Y | 0-15% | 0-10% | 0-8% | 0-5% |
| Zr | 0-15% | 0-10% | 0-8% | 0-5% |
| Ag | 0-15% | 0-10% | 0-8% | 0-5% |
| Al | 0-15% | 0-10% | 0-8% | 0-5% |
| Co | 0-15% | 0-10% | 0-8% | 0-5% |
| Fe | 0-15% | 0-10% | 0-8% | 0-5% |
| Mg | 0-15% | 0-10% | 0-8% | 0-5% |
| Ni | 0-15% | 0-10% | 0-8% | 0-5% |
| Pt | 0-15% | 0-10% | 0-8% | 0-5% |
| Si | 0-15% | 0-10% | 0-8% | 0-5% |
| Sn | 0-15% | 0-10% | 0-8% | 0-5% |
| Cs₂O | 0-1% | 0-1% | 0-1% | 0-1% |
| La₂O₃ | 0-1% | 0-1% | 0-1% | 0-1% |
| Y₂O₃ | 0-1% | 0-1% | 0-1% | 0-1% |
| ZrO₂ | 0-1% | 0-1% | 0-1% | 0-1% |
| C | <0.06% | <0.06% | <0.06% | <0.06% |
| N | <0.06% | <0.06% | <0.06% | <0.06% |
| O | <0.06% | <0.06% | <0.06% | <0.06% |

| Component/Wt. % | Ex. 111 | Ex. 112 | Ex. 113 | Ex. 114 |
|---|---|---|---|---|
| Mo | 40-95% | 40-95% | 40-95% | 40-95% |
| C | 0.01-0.3% | 0-0.3% | 0-0.3% | 0-0.3% |
| Co | ≤0.002% | ≤0.002% | ≤0.002% | ≤0.002% |
| Cs₂O | 0-0.2% | 0-0.2% | 0.01-0.2% | 0-0.2% |
| Fe | ≤0.02% | ≤0.02% | ≤0.02% | ≤0.02% |
| H | ≤0.002% | ≤0.002% | ≤0.002% | ≤0.002% |
| Hf | 0.1-2.5% | 0-2.5% | 0-2.5% | 0-2.5% |
| O | ≤0.06% | ≤0.06% | ≤0.06% | ≤0.06% |
| Os | ≤1% | ≤1% | ≤1% | ≤1% |
| La₂O₃ | 0-2% | 0.1-2% | 0-2% | 0-2% |
| N | ≤20 ppm | ≤20 ppm | ≤20 ppm | ≤20 ppm |
| Nb | ≤0.01% | ≤0.01% | ≤0.01% | ≤0.01% |
| Pt | ≤1% | ≤1% | ≤1% | ≤1% |
| Re | 5-40% | 5-40% | 5-40% | 5-40% |
| S | ≤0.008% | ≤0.008% | ≤0.008% | ≤0.008% |
| Sn | ≤0.002% | ≤0.002% | ≤0.002% | ≤0.002% |
| Ta | 0-50% | 0-50% | 0-50% | 0-50% |
| Tc | ≤1% | ≤1% | ≤1% | ≤1% |
| Ti | ≤1% | ≤1% | ≤1% | ≤1% |
| V | ≤1% | ≤1% | ≤1% | ≤1% |
| W | 0-50% | 0-50% | 0-50% | 0.5-50% |
| Y₂O₃ | 0-1% | 0-1% | 0.1-1% | 0-1% |
| Zr | ≤1% | ≤1% | ≤1% | ≤1% |
| ZrO₂ | 0-3% | 0-3% | 0-3% | 0-3% |
| Ag | 0-5% | 0-5% | 0-5% | 0-5% |
| Al | 0-5% | 0-5% | 0-5% | 0-5% |
| Co | 0-5% | 0-5% | 0-5% | 0-5% |
| Mg | 0-5% | 0-5% | 0-5% | 0-5% |
| Ni | 0-5% | 0-5% | 0-5% | 0-5% |
| Si | 0-5% | 0-5% | 0-5% | 0-5% |
| Sn | 0-5% | 0-5% | 0-5% | 0-5% |
| CNT | 0-10% | 0-10% | 0-10% | 0-10% |

| Component/Wt. % | Ex. 115 | Ex. 116 | Ex. 117 |
|---|---|---|---|
| Mo | 40-95% | 40-95% | 40-95% |
| C | 0-0.3% | 0-0.3% | 0-0.3% |
| Co | ≤0.002% | ≤0.002% | ≤0.002% |
| Cs₂O | 0-0.2% | 0-0.2% | 0-0.2% |
| H | ≤0.002% | ≤0.002% | ≤0.002% |
| Hf | 0-2.5% | 0-2.5% | 0-2.5% |
| O | ≤0.06% | ≤0.06% | ≤0.06% |
| Os | ≤1% | ≤1% | ≤1% |
| La₂O₃ | 0-2% | 0-2% | 0-2% |
| N | ≤20 ppm | ≤20 ppm | ≤20 ppm |
| Nb | ≤0.01% | ≤0.01% | ≤0.01% |
| Pt | ≤1% | ≤1% | ≤1% |
| Re | 5-40% | 5-40% | 5-40% |
| S | ≤0.008% | ≤0.008% | ≤0.008% |
| Sn | ≤0.002% | ≤0.002% | ≤0.002% |
| Ta | 0-50% | 0.5-50% | 0-50% |
| Tc | ≤1% | ≤1% | ≤1% |
| Ti | ≤1% | ≤1% | ≤1% |
| V | ≤1% | ≤1% | ≤1% |
| W | 0-50% | 0-50% | 0-50% |
| Y₂O₃ | 0-1% | 0-1% | 0-1% |
| ZrO₂ | 0.1-3% | 0-3% | 0-3% |
| Ag | 0-5% | 0-5% | 0-5% |
| Al | 0-5% | 0-5% | 0-5% |
| Fe | 0-5% | 0-5% | 0-5% |
| Mg | 0-5% | 0-5% | 0-5% |
| Ni | 0-5% | 0-5% | 0-5% |
| Si | 0-5% | 0-5% | 0-5% |
| CNT | 0-10% | 0-10% | 0-10% |
| Component/Wt. % | Ex. 118 | Ex. 119 | Ex. 120 |
| Mo | 60-95% | 60-95% | 60-90% |
| C | 0-0.3% | 0-0.3% | 0-0.3% |
| Co | ≤0.002% | ≤0.002% | ≤0.002% |
| Cs₂O | 0-0.2% | 0-0.2% | 0-0.2% |
| H | ≤0.002% | ≤0.002% | ≤0.002% |
| Hf | 0-2.5% | 0-2.5% | 0-2.5% |
| O | ≤0.06% | ≤0.06% | ≤0.06% |
| Os | ≤1% | ≤1% | ≤1% |
| La₂O₃ | 0-2% | 0-2% | 0-2% |
| N | ≤20 ppm | ≤20 ppm | ≤20 ppm |
| Nb | ≤0.01% | ≤0.01% | ≤0.01% |
| Pt | ≤1% | ≤1% | ≤1% |
| Re | 5-40% | 5-40% | 10-40% |
| S | ≤0.008% | ≤0.008% | ≤0.008% |
| Sn | ≤0.002% | ≤0.002% | ≤0.002% |
| Ta | 0-50% | 0.5-50% | 0-50% |
| Tc | ≤1% | ≤1% | ≤1% |
| Ti | ≤1% | ≤1% | ≤1% |
| V | ≤1% | ≤1% | ≤1% |
| W | 0-50% | 0-50% | 0-50% |
| Y₂O₃ | 0-1% | 0-1% | 0-1% |
| ZrO₂ | 0.1-3% | 0-3% | 0-3% |
| Ag | 0-5% | 0-5% | 0-5% |
| Al | 0-5% | 0-5% | 0-5% |
| Fe | 0-5% | 0-5% | 0-5% |
| Mg | 0-5% | 0-5% | 0-5% |
| Ni | 0-5% | 0-5% | 0-5% |
| Si | 0-5% | 0-5% | 0-5% |
| CNT | 0-10% | 0-10% | 0-10% |

| Component/Wt. % | Ex. 121 | Ex. 122 | Ex. 123 | Ex. 124 |
|---|---|---|---|---|
| Mo | 60-95% | 60-95% | 50-95% | 40-80% |
| C | 0.05-0.15% | 0-0.15% | 0-0.15% | 0-0.15% |
| Cs₂O | 0-0.2% | 0-0.2% | 0.04-0.1% | 0-0.2% |
| Hf | 0.8-1.4% | 0-2% | 0-2.5% | 0-2.5% |
| La₂O₃ | 0-2% | 0.3-0.7% | 0-2% | 0-2% |
| Re | 5-40% | 5-40% | 5-40% | 5-40% |
| Ta | 0-2% | 0-2% | 0-50% | 0-50% |
| W | 0-2% | 0-2% | 0-50% | 20-50% |
| Y₂O₃ | 0-1% | 0-1% | 0.3-0.5% | 0-1% |
| ZrO₂ | 0-3% | 0-3% | 0-3% | 0-3% |

| Component/Wt. % | Ex. 125 | Ex. 126 | Ex. 127 |
|---|---|---|---|
| Mo | 97-95% | 50-90% | 60-95% |
| C | 0-0.15% | 0-0.15% | 0-0.15% |
| Cs₂O | 0-0.2% | 0-0.2% | 0-0.2% |
| Hf | 0-2.5% | 0-2.5% | 0-2.5% |
| La₂O₃ | 0-2% | 0-2% | 0-2% |
| Re | 5-30 | 5-40% | 5-40% |
| Ta | 0-3% | 10-50% | 0-40% |
| W | 0-3% | 0-50% | 0-40% |
| Y₂O₃ | 0-1% | 0-1% | 0-1% |
| ZrO₂ | 1.2-1.8% | 0-3% | 0-3% |
| Component/Wt. % | Ex. 128 | Ex. 129 | Ex. 130 |
| W | 20-95% | 60-95% | 20-80% |
| Re | 5-47.5% | 5-40% | 5-47.5% |
| Mo | 0-47.5% | <0.5% | 1-47.5% |
| Cu | <0.5% | <0.5% | <0.5% |
| C | ≤0.15% | ≤0.15% | ≤0.15% |
| Co | ≤0.002% | ≤0.002% | ≤0.002% |
| Cs₂O | ≤0.2% | ≤0.2% | ≤0.2% |
| Fe | ≤0.02% | ≤0.02% | ≤0.02% |
| H | ≤0.002% | ≤0.002% | ≤0.002% |
| Hf | <0.5% | <0.5% | <0.5% |
| La₂O₃ | <0.5% | <0.5% | <0.5% |
| O | ≤0.06% | ≤0.06% | ≤0.06% |
| Os | <0.5% | <0.5% | <0.5% |
| N | ≤20 ppm | ≤20 ppm | ≤20 ppm |
| Nb | ≤0.01% | ≤0.01% | ≤0.01% |
| Pt | <0.5% | <0.5% | <0.5% |
| S | ≤0.008% | ≤0.008% | ≤0.008% |
| Sn | ≤0.002% | ≤0.002% | ≤0.002% |
| Ta | <0.5% | <0.5% | <0.5% |
| Tc | <0.5% | <0.5% | <0.5% |
| Ti | <0.5% | <0.5% | <0.5% |
| V | <0.5% | <0.5% | <0.5% |
| Y₂O₃ | <0.5% | <0.5% | <0.5% |
| Zr | <0.5% | <0.5% | <0.5% |
| ZrO₂ | <0.5% | <0.5% | <0.5% |
| Ag | 0-5% | 0-5% | 0-5% |
| Al | 0-5% | 0-5% | 0-5% |
| Mg | 0-5% | 0-5% | 0-5% |
| Ni | 0-5% | 0-5% | 0-5% |
| Si | 0-5% | 0-5% | 0-5% |
| CNT | 0-10% | 0-10% | <0.5%. |

| Component/Wt. % | Ex. 131 | Ex. 132 | Ex. 133 | Ex. 134 |
|---|---|---|---|---|
| W | 1-94.9% | 1-94.9% | 1-94.9% | 10-95% |
| Cu | 0.1-94% | 0.1-94% | 0.1-94% | 1-84% |
| C | 0.01-0.3% | 0-0.3% | 0-0.3% | 0-0.3% |
| Co | ≤0.002% | ≤0.002% | ≤0.002% | ≤0.002% |
| Cs₂O | 0-0.2% | 0-0.2% | 0.01-0.2% | 0-0.2% |
| Fe | ≤0.02% | ≤0.02% | ≤0.02% | ≤0.02% |
| H | ≤0.002% | ≤0.002% | ≤0.002% | ≤0.002% |
| Hf | 0.1-2.5% | 0-2.5% | 0-2.5% | 0-2.5% |
| O | ≤0.06% | ≤0.06% | ≤0.06% | ≤0.06% |
| Os | ≤1% | ≤1% | ≤1% | ≤1% |
| La₂O₃ | 0-2% | 0.1-2% | 0-2% | 0-2% |
| Mo | 0-5% | 0.1-3% | 0-2% | 0-3% |
| N | ≤20 ppm | ≤20 ppm | ≤20 ppm | ≤20 ppm |
| Nb | ≤0.01% | ≤0.01% | ≤0.01% | ≤0.01% |
| Pt | ≤1% | ≤1% | ≤1% | ≤1% |
| Re | 5-40% | 5-40% | 5-40% | 6-40% |
| S | ≤0.008% | ≤0.008% | ≤0.008% | ≤0.008% |
| Sn | ≤0.002% | ≤0.002% | ≤0.002% | ≤0.002% |
| Ta | 0-50% | 0-50% | 0-50% | 0-50% |
| Tc | ≤1% | ≤1% | ≤1% | ≤1% |
| Ti | ≤1% | ≤1% | ≤1% | ≤1% |
| V | ≤1% | ≤1% | ≤1% | ≤1% |
| Y₂O₃ | 0-1% | 0-1% | 0.1-1% | 0-1% |
| Zr | ≤1% | ≤1% | ≤1% | ≤1% |
| ZrO₂ | 0-3% | 0-3% | 0-3% | 0-3% |
| Ag | 0-5% | 0-5% | 0-5% | 0-5% |
| Al | 0-5% | 0-5% | 0-5% | 0-5% |
| Fe | 0-5% | 0-5% | 0-5% | 0-5% |
| Mg | 0-5% | 0-5% | 0-5% | 0-5% |
| Ni | 0-5% | 0-5% | 0-5% | 0-5% |
| Si | 0-5% | 0-5% | 0-5% | 0-5% |
| CNT | 0-10% | 0-10% | 0-10% | 0-10% |

| Component/Wt. % | Ex. 135 | Ex. 136 | Ex. 137 |
|---|---|---|---|
| W | 20-96% | 25-92% | 30-88% |
| Cu | 2-74% | 2-68% | 5-62% |
| C | 0-0.3% | 0-0.3% | 0-0.3% |
| Co | ≤0.002% | ≤0.002% | ≤0.002% |
| Cs₂O | 0-0.2% | 0-0.2% | 0-0.2% |
| H | ≤0.002% | ≤0.002% | ≤0.002% |
| Hf | 0-2.5% | 0-2.5% | 0-2.5% |
| O | ≤0.06% | ≤0.06% | ≤0.06% |
| Os | ≤1% | ≤1% | ≤1% |
| La₂O₃ | 0-2% | 0-2% | 0-2% |
| Mo | 0-3% | 0-2% | 0-1% |
| N | ≤20 ppm | ≤20 ppm | ≤20 ppm |
| Nb | ≤0.01% | ≤0.01% | ≤0.01% |
| Pt | ≤1% | ≤1% | ≤1% |
| Re | 6-40% | 7-40% | 8-40% |
| S | ≤0.008% | ≤0.008% | ≤0.008% |
| Sn | ≤0.002% | ≤0.002% | ≤0.002% |
| Ta | 0-50% | 0.5-50% | 0-50% |
| Tc | ≤1% | ≤1% | ≤1% |
| Ti | ≤1% | ≤1% | ≤1% |
| V | ≤1% | ≤1% | ≤1% |
| Y₂O₃ | 0-1% | 0-1% | 0-1% |
| ZrO₂ | 0.1-3% | 0-3% | 0-3% |
| Ag | 0-5% | 0-5% | 0-5% |
| Al | 0-5% | 0-5% | 0-5% |
| Fe | 0-5% | 0-5% | 0-5% |
| Mg | 0-5% | 0-5% | 0-5% |
| Ni | 0-5% | 0-5% | 0-5% |
| Si | 0-5% | 0-5% | 0-5% |
| CNT | 0-10% | 0-10% | 0-10% |

| Component/Wt. % | Ex. 138 | Ex. 139 | Ex. 140 | Ex. 141 |
|---|---|---|---|---|
| W | 25-88% | 35-87% | 40-86% | 50-85% |
| Cu | 5-68% | 5-57% | 5-51% | 5-40% |
| C | 0.05-0.15% | 0-0.15% | 0-0.15% | 0-0.15% |
| Cs₂O | 0-0.2% | 0-0.2% | 0.04-0.1% | 0-0.2% |
| Hf | 0.8-1.4% | 0-2.5% | 0-2.5% | 0-2.5% |
| La₂O₃ | 7-20% | 8-20% | 9-20% | 10-20% |
| Re | 0-40% | 0-40% | 0-40% | 0-40% |
| Ta | 0-50% | 0-50% | 0-50% | 0-50% |
| Y₂O₃ | 0-1% | 0-1% | 0.3-0.5% | 0-1% |
| ZrO₂ | 0-3% | 0-3% | 0-3% | 0-3% |

| Component/Wt. % | Ex. 142 | Ex. 143 | Ex. 144 |
|---|---|---|---|
| W | 55-88% | 60-87% | 70-86% |
| Cu | 1-34% | 1-28% | 1-17% |
| C | 0-0.15% | 0-0.15% | 0-0.15% |
| Cs₂O | 0-0.2% | 0-0.2% | 0-0.2% |
| Hf | 0-2.5% | 0-2.5% | 0-2.5% |
| La₂O₃ | 0-2% | 0-2% | 0-2% |
| Re | 11-40% | 12-40% | 13-40% |
| Ta | 0-50% | 10-50% | 0-50% |
| W | 0-50% | 0-50% | 0-50% |
| Y₂O₃ | 0-1% | 0-1% | 0-1% |
| ZrO₂ | 1.2-1.8% | 0-3% | 0-3% |
| Component/Wt. % | Ex. 145 | Ex. 146 | Ex. 147 |
| Ti | 55-66% | 65-76% | 70-76% |
| Mo | 20-41% | 20-31% | 20-26% |
| Re | 4-20% | 4-20% | 4-20% |
| Yt | <0.5% | <0.5% | <0.5% |
| Nb | <0.5% | <0.5% | <0.5% |
| Co | <0.5% | <0.5% | <0.5% |
| Cr | <0.5% | <0.5% | <0.5% |
| Zr | <0.5% | <0.5% | <0.5% |
| C | ≤0.15% | ≤0.15% | ≤0.15% |
| O | ≤0.06% | ≤0.06% | ≤0.06% |
| N | ≤20 ppm | ≤20 ppm | ≤20 ppm |
| Component/Wt. % | Ex. 148 | Ex. 149 | Ex. 150 |
| W | 20-95% | 60-85% | 20-84% |
| Re | 5-47.5% | 15-40% | 5-47.5% |
| Mo | 0-47.5% | <0.5% | 1-47.5% |
| Component/Wt. % | Ex. 151 | Ex. 152 | Ex. 153 |
| W | 50.1-93% | 65-92% | 70-90% |
| Re | 7-40% | 8-35% | 9-30% |
| Mo | 0-40% | <0.5% | 1-30% |

| Component/Wt. % | Ex. 154 | Ex. 155 | Ex. 156 |
|---|---|---|---|
| W | 20-49% | 20-49% | 20-49% |
| Re | 5-40% | 5-40% | 5-39% |
| Mo | 20-60% | 30-60% | 40-60% |

| Component/Wt. % | Ex. 157 | Ex. 158 | Ex. 159 |
|---|---|---|---|
| W | 20-40% | 20-35% | 20-30% |
| Re | 7-40% | 10-40% | 25-40% |
| Mo | 0-40% | 10-40% | 25-40% |

| Component/Wt. % | Ex. 160 | Ex. 161 | Ex. 162 |
|---|---|---|---|
| W | 20-95% | 60-93% | 20-80% |
| Re | 5-47.5% | 7-40% | 5-47.5% |
| Mo | 0-47.5% | <0.5% | 1-47.5% |
| Cu | <0.5% | <0.5% | <0.5% |
| C | ≤0.15% | ≤0.15% | ≤0.15% |
| Co | ≤0.002% | ≤0.002% | ≤0.002% |
| Cs₂O | ≤0.2% | ≤0.2% | ≤0.2% |
| Fe | ≤0.02% | ≤0.02% | ≤0.02% |
| H | ≤0.002% | ≤0.002% | ≤0.002% |
| Hf | <0.5% | <0.5% | <0.5% |
| La₂O₃ | <0.5% | <0.5% | <0.5% |
| O | ≤0.06% | ≤0.06% | ≤0.06% |
| Os | <0.5% | <0.5% | <0.5% |
| N | ≤20 ppm | ≤20 ppm | ≤20 ppm |
| Nb | ≤0.01% | ≤0.01% | ≤0.01% |
| Pt | <0.5% | <0.5% | <0.5% |
| S | ≤0.008% | ≤0.008% | ≤0.008% |
| Sn | ≤0.002% | ≤0.002% | ≤0.002% |
| Ta | <0.5% | <0.5% | <0.5% |
| Tc | <0.5% | <0.5% | <0.5% |
| Ti | <0.5% | <0.5% | <0.5% |
| V | <0.5% | <0.5% | <0.5% |
| Y₂O₃ | <0.5% | <0.5% | <0.5% |
| Zr | <0.5% | <0.5% | <0.5% |
| ZrO₂ | <0.5% | <0.5% | <0.5% |
| Ag | 0-5% | 0-5% | 0-5% |
| Al | 0-5% | 0-5% | 0-5% |
| Fe | 0-5% | 0-5% | 0-5% |
| Mg | 0-5% | 0-5% | 0-5% |
| Ni | 0-5% | 0-5% | 0-5% |
| Si | 0-5% | 0-5% | 0-5% |
| CNT | 0-10% | 0-10% | <0.5% |

| Component/Wt. % | Ex. 163 | Ex. 164 | Ex. 165 | Ex. 166 |
|---|---|---|---|---|
| Ag | 0-10% | 0-10% | 0-10% | 0-10% |
| Al | 0-10% | 0-10% | 0-10% | 2-10% |
| B | 0-10% | 0-10% | 0-10% | 0-10% |
| Bi | 0-10% | 0-10% | 0-10% | 0-10% |
| Cr | 2-30% | 10-30% | 0-20% | 0-20% |
| Cu | 0-10% | 0-10% | 0-10% | 0-10% |
| Co | 0-10% | 32-70% | 0-10% | 0-10% |
| Fe | 50-80% | 0-20% | 0-10% | 0-10% |
| Hf | 0-10% | 0-10% | 0-10% | 0-10% |
| Ir | 0-10% | 0-10% | 0-10% | 0-10% |
| La | 0-10% | 0-10% | 0-10% | 0-10% |
| Mg | 0-10% | 0-10% | 0-10% | 0-10% |
| Mn | 0-20% | 0-10% | 0-10% | 0-10% |
| Mo | 0-10% | 0-30% | 0-16% | 0-16% |
| Nb | 0-10% | 0-10% | 0-10% | 0-10% |
| Ni | 0.1-30% | 0.1-40% | 0-10% | 0-10% |
| Os | 0-10% | 0-10% | 0-10% | 0-10% |
| Pt | 0-10% | 0-10% | 0-10% | 0-10% |
| Re | 5-40% | 4.8-40% | 4.5-80% | 4.5-80% |
| Rh | 0-10% | 0-10% | 0-10% | 0-10% |
| Se | 0-10% | 0-10% | 0-10% | 0-10% |
| Si | 0-10% | 0-10% | 0-10% | 0-10% |
| Sn | 0-10% | 0-10% | 0-12% | 0-12% |
| Ta | 0-10% | 0-10% | 0-10% | 0-10% |
| Tc | 0-10% | 0-10% | 0-10% | 0-10% |
| Ti | 0-10% | 0-10% | 70-91.5% | 70-91.5% |
| V | 0-10% | 0-10% | 0-10% | 0.01-10% |
| W | 0-10% | 0-20% | 0-10% | 0-10% |
| Y | 0-10% | 0-10% | 0-10% | 0-10% |
| Zr | 0-10% | 0-10% | 0-10% | 0-10% |
| Cs₂O | 0-1% | 0-1% | 0-1% | 0-1% |
| La₂O₃ | 0-3% | 0.1-2% | 0-2% | 0-2% |
| Y₂O₃ | 0-1% | 0-1% | 0.1-1% | 0-1% |
| ZrO₂ | 0-3% | 0-3% | 0-3% | 0-3% |
| C | <0.06% | <0.06% | <0.06% | <0.06% |
| N | <0.06% | <0.06% | <0.06% | <0.06% |
| O | <0.06% | <0.06% | <0.06% | <0.06% |

| Component/Wt. % | Ex. 167 | Ex. 168 | Ex. 169 | Ex. 170 |
|---|---|---|---|---|
| Ag | 0-10% | 0-10% | 0-10% | 0-10% |
| Al | 0-10% | 0-10% | 0-10% | 0-10% |
| B | 0-10% | 0-10% | 0-10% | 0-10% |
| Bi | 0-10% | 0-10% | 0-10% | 0-10% |
| Cr | 0-10% | 0-20% | 0-20% | 0-10% |
| Cu | 0-10% | 0-10% | 0-50% | 0-10% |
| Co | 0-10% | 0-10% | 0-10% | 0-10% |
| Fe | 0-10% | 0-10% | 0-10% | 0-10% |
| Hf | 0-10% | 0-10% | 0-10% | 0-10% |
| Ir | 0-10% | 0-10% | 0-10% | 0-12% |
| La | 0-10% | 0-10% | 0-10% | 0-10% |
| Mg | 0-10% | 0-10% | 0-10% | 0-10% |
| Mn | 0-10% | 0-10% | 0-10% | 0-10% |
| Mo | 0-55% | 40-93% | 0-50% | 0-20% |
| Nb | 0-10% | 0-10% | 0-10% | 40-85% |
| Ni | 0-45% | 0-10% | 0-10% | 0-10% |
| Os | 0-10% | 0-10% | 0-10% | 0-10% |
| Pt | 0-10% | 0-10% | 0-10% | 0-10% |
| Re | 14-40% | 7-40% | 7-40% | 7-40% |
| Rh | 0-10% | 0-10% | 0-10% | 0-10% |
| Se | 0-10% | 0-10% | 0-10% | 0-10% |
| Si | 0-10% | 0-10% | 0-10% | 0-10% |
| Sn | 0-10% | 0-10% | 0-10% | 0-10% |
| Ta | 35-84% | 0-50% | 0-50% | 0-35% |
| Tc | 0-10% | 0-10% | 0-10% | 0-10% |
| Ti | 0-10% | 0-10% | 0-10% | 0-10% |
| V | 0-10% | 0-10% | 0-10% | 0-10% |
| W | 0.1-25% | 0-50% | 14-10% | 0-15% |
| Y | 0-10% | 0-10% | 0-10% | 0-10% |
| Zr | 0-10% | 0-10% | 0-50% | 0-10% |
| Cs₂O | 0-1% | 0-1% | 0-1% | 0-1% |
| La₂O₃ | 0-3% | 0.1-2% | 0-2% | 0-2% |
| Y₂O₃ | 0-1% | 0-1% | 0.1-1% | 0-1% |
| ZrO₂ | 0-3% | 0-3% | 0-3% | 0-3% |
| C | <0.06% | <0.06% | <0.06% | <0.06% |
| N | <0.06% | <0.06% | <0.06% | <0.06% |
| O | <0.06% | <0.06% | <0.06% | <0.06% |

| Component/Wt. % | Ex. 171 | Ex. 172 | Ex. 173 | Ex. 174 |
|---|---|---|---|---|
| Ag | 0-10% | 0-10% | 0-5% | 0-5% |
| Al | 0-10% | 0-10% | 0-5% | 5-7% |
| B | 0-10% | 0-10% | 0-5% | 0-5% |
| Bi | 0-10% | 0-10% | 0-5% | 0-5% |
| Cr | 0-10% | 1-95% | 12-28% | 0-5% |
| Cu | 0-10% | 0-10% | 0-5% | 0-5% |
| Co | 0-10% | 0-10% | 36-68% | 0-5% |
| Fe | 0-10% | 0-10% | 0-18% | 0-5% |
| Hf | 0-10% | 0-10% | 0-5% | 0-5% |
| Ir | 0-10% | 0-10% | 0-5% | 0-5% |
| La | 0-10% | 0-10% | 0-5% | 0-5% |
| Mg | 0-10% | 0-10% | 0-5% | 0-5% |
| Mn | 0-10% | 0-10% | 0-5% | 0-5% |
| Mo | 0-10% | 0-20% | 0-12% | 0-5% |
| Nb | 0-10% | 0-10% | 0-5% | 0-5% |
| Ni | 30-58% | 0-10% | 9-36% | 0-5% |
| Os | 0-10% | 0-10% | 0-5% | 0-5% |
| Pt | 0-10% | 0-10% | 0-5% | 0-5% |
| Re | 5-40% | 5-40% | 4.8-40% | 4.5-40% |
| Rh | 0-10% | 0-10% | 0-5% | 0-5% |
| Se | 0-10% | 0-10% | 0-5% | 0-5% |
| Si | 0-10% | 0-10% | 0-5% | 0-5% |
| Sn | 0-10% | 0-10% | 0-5% | 0-5% |
| Ta | 0-10% | 0-10% | 0-5% | 0-5% |
| Tc | 0-10% | 0-10% | 0-5% | 0-5% |
| Ti | 30-58% | 0-40% | 0-5% | 70-91.5% |
| V | 0-10% | 0-10% | 0-5% | 3-6% |
| W | 0-10% | 0-10% | 0-16% | 0-5% |
| Y | 0-10% | 0-10% | 0-5% | 0-5% |
| Zr | 0-10% | 0-20% | 0-5% | 0-5% |
| Cs₂O | 0-1% | 0-1% | 0-1% | 0-1% |
| La₂O₃ | 0-3% | 0.1-2% | 0-2% | 0-2% |
| Y₂O₃ | 0-1% | 0-1% | 0.1-1% | 0-1% |
| ZrO₂ | 0-3% | 0-3% | 0-3% | 0-3% |
| C | <0.06% | <0.06% | <0.06% | <0.06% |
| N | <0.06% | <0.06% | <0.06% | <0.06% |
| O | <0.06% | <0.06% | <0.06% | <0.06% |

| Component/Wt. % | Ex. 175 | Ex. 176 | Ex. 177 | Ex. 178 |
|---|---|---|---|---|
| Ag | 0-8% | 0-8% | 0-8% | 0-8% |
| Al | 0-8% | 0-8% | 0-8% | 2-10% |
| B | 0-8% | 0-8% | 0-8% | 0-8% |
| Bi | 0-8% | 0-8% | 0-8% | 0-8% |
| Cr | 2-30% | 10-30% | 0-20% | 0-20% |
| Cu | 0-8% | 0-8% | 0-8% | 0-8% |
| Co | 0-8% | 32-70% | 0-8% | 0-8% |
| Fe | 50-80% | 0-20% | 0-8% | 0-8% |
| Hf | 0-8% | 0-8% | 0-8% | 0-8% |
| Ir | 0-8% | 0-8% | 0-8% | 0-8% |
| La | 0-8% | 0-8% | 0-8% | 0-8% |
| Mg | 0-8% | 0-8% | 0-8% | 0-8% |
| Mn | 0-20% | 0-8% | 0-8% | 0-8% |
| Mo | 0-8% | 0-30% | 0-16% | 0-16% |
| Nb | 0-8% | 0-8% | 0-8% | 0-8% |
| Ni | 0.1-30% | 0.1-40% | 0-8% | 0-8% |
| Os | 0-8% | 0-8% | 0-8% | 0-8% |
| Pt | 0-8% | 0-8% | 0-8% | 0-8% |
| Re | 5-40% | 4.8-40% | 4.5-80% | 4.5-80% |
| Rh | 0-8% | 0-8% | 0-8% | 0-8% |
| Se | 0-8% | 0-8% | 0-8% | 0-8% |
| Si | 0-8% | 0-8% | 0-8% | 0-8% |
| Sn | 0-8% | 0-8% | 0-12% | 0-12% |
| Ta | 0-8% | 0-8% | 0-8% | 0-8% |
| Tc | 0-8% | 0-8% | 0-8% | 0-8% |
| Ti | 0-8% | 0-8% | 70-91.5% | 70-91.5% |
| V | 0-8% | 0-8% | 0-8% | 0.01-10% |
| W | 0-8% | 0-20% | 0-8% | 0-8% |
| Y | 0-8% | 0-8% | 0-8% | 0-8% |
| Zr | 0-8% | 0-8% | 0-8% | 0-8% |

| Component/Wt. % | Ex. 179 | Ex. 180 | Ex. 181 | Ex. 182 |
|---|---|---|---|---|
| Ag | 0-8% | 0-8% | 0-8% | 0-8% |
| Al | 0-8% | 0-8% | 0-8% | 0-8% |
| B | 0-8% | 0-8% | 0-8% | 0-8% |
| Bi | 0-8% | 0-8% | 0-8% | 0-8% |
| Cr | 0-8% | 0-20% | 0-20% | 0-8% |
| Cu | 0-8% | 0-8% | 0-50% | 0-8% |
| Co | 0-8% | 0-8% | 0-8% | 0-8% |
| Fe | 0-8% | 0-8% | 0-8% | 0-8% |
| Hf | 0-8% | 0-8% | 0-8% | 0-8% |
| Ir | 0-8% | 0-8% | 0-8% | 0-12% |
| La | 0-8% | 0-8% | 0-8% | 0-8% |
| Mg | 0-8% | 0-8% | 0-8% | 0-8% |
| Mn | 0-8% | 0-8% | 0-8% | 0-8% |
| Mo | 0-55% | 40-93% | 0-50% | 0-20% |
| Nb | 0-8% | 0-8% | 0-8% | 40-85% |
| Ni | 0-45% | 0-8% | 0-8% | 0-8% |
| Os | 0-8% | 0-8% | 0-8% | 0-8% |
| Pt | 0-8% | 0-8% | 0-8% | 0-8% |
| Re | 14-40% | 7-40% | 7-40% | 7-40% |
| Rh | 0-8% | 0-8% | 0-8% | 0-8% |
| Se | 0-8% | 0-8% | 0-8% | 0-8% |
| Si | 0-8% | 0-8% | 0-8% | 0-8% |
| Sn | 0-8% | 0-8% | 0-8% | 0-8% |
| Ta | 35-84% | 0-50% | 0-50% | 0-35% |
| Tc | 0-8% | 0-8% | 0-8% | 0-8% |
| Ti | 0-8% | 0-8% | 0-8% | 0-8% |
| V | 0-8% | 0-8% | 0-8% | 0-8% |
| W | 0.1-25% | 0-50% | 14-10% | 0-15% |
| Y | 0-8% | 0-8% | 0-8% | 0-8% |
| Zr | 0-8% | 0-8% | 0-50% | 0-8% |

| Component/Wt. % | Ex. 183 | Ex. 184 | Ex. 185 | Ex. 186 |
|---|---|---|---|---|
| Ag | 0-5% | 0-5% | 0-5% | 0-5% |
| Al | 0-5% | 0-5% | 0-5% | 5-7% |
| B | 0-5% | 0-5% | 0-5% | 0-5% |
| Bi | 0-5% | 0-5% | 0-5% | 0-5% |
| Cr | 0-5% | 1-95% | 12-28% | 0-5% |
| Cu | 0-5% | 0-5% | 0-5% | 0-5% |
| Co | 0-5% | 0-5% | 36-68% | 0-5% |
| Fe | 0-5% | 0-5% | 0-18% | 0-5% |
| Hf | 0-5% | 0-5% | 0-5% | 0-5% |
| Ir | 0-5% | 0-5% | 0-5% | 0-5% |
| La | 0-5% | 0-5% | 0-5% | 0-5% |
| Mg | 0-5% | 0-5% | 0-5% | 0-5% |
| Mn | 0-5% | 0-5% | 0-5% | 0-5% |
| Mo | 0-5% | 0-20% | 0-12% | 0-5% |
| Nb | 0-5% | 0-5% | 0-5% | 0-5% |
| Ni | 30-58% | 0-5% | 9-36% | 0-5% |
| Os | 0-5% | 0-5% | 0-5% | 0-5% |
| Pt | 0-5% | 0-5% | 0-5% | 0-5% |
| Re | 5-40% | 5-40% | 4.8-40% | 4.5-40% |
| Rh | 0-5% | 0-5% | 0-5% | 0-5% |
| Se | 0-5% | 0-5% | 0-5% | 0-5% |
| Si | 0-5% | 0-5% | 0-5% | 0-5% |
| Sn | 0-5% | 0-5% | 0-5% | 0-5% |
| Ta | 0-5% | 0-5% | 0-5% | 0-5% |
| Tc | 0-5% | 0-5% | 0-5% | 0-5% |
| Ti | 30-58% | 0-40% | 0-5% | 70-91.5% |
| V | 0-5% | 0-5% | 0-5% | 3-6% |
| W | 0-5% | 0-5% | 0-16% | 0-5% |
| Y | 0-5% | 0-5% | 0-5% | 0-5% |
| Zr | 0-5% | 0-20% | 0-5% | 0-5% |

| Component/Wt. % | Ex. 187 | Ex. 188 | Ex. 189 | Ex. 190 |
|---|---|---|---|---|
| Ag | 0-5% | 0-5% | 0-5% | 0-5% |
| Al | 0-5% | 0-5% | 0-5% | 0-5% |
| B | 0-5% | 0-5% | 0-5% | 0-5% |
| Bi | 0-5% | 0-5% | 0-5% | 0-5% |
| Cr | 0-5% | 0-5% | 0-5% | 0-5% |
| Cu | 0-5% | 0-5% | 0-5% | 0-5% |
| Co | 0-5% | 0-5% | 0-5% | 0-5% |
| Fe | 0-5% | 0-5% | 0-5% | 0-5% |
| Hf | 0-5% | 0-5% | 0-5% | 0-5% |
| Ir | 0-5% | 0-5% | 0-5% | 0-5% |
| La | 0-5% | 0-5% | 0-5% | 0-5% |
| Mg | 0-5% | 0-5% | 0-5% | 0-5% |
| Mn | 0-5% | 0-5% | 0-5% | 0-5% |
| Mo | 1-15% | 2-10% | 3-8% | 0-5% |
| Nb | 0-5% | 0-5% | 0-5% | 20-45% |
| Ni | 0-5% | 0-5% | 0-5% | 0-5% |
| Os | 0-5% | 0-5% | 0-5% | 0-5% |
| Pt | 0-5% | 0-5% | 0-5% | 0-5% |
| Re | 0-5% | 0-5% | 0-5% | 0-5% |
| Rh | 0-5% | 0-5% | 0-5% | 0-5% |
| Se | 0-5% | 0-5% | 0-5% | 0-5% |
| Si | 0-5% | 0-5% | 0-5% | 0-5% |
| Sn | 0-5% | 0-5% | 0-5% | 0-5% |
| Ta | 0-5% | 0-5% | 0-5% | 1-15% |
| Tc | 0-5% | 0-5% | 0-5% | 0-5% |
| Ti | 51-70% | 51-70% | 55-70% | 51-70% |
| V | 0-5% | 0-5% | 0-5% | 0-5% |
| W | 0-5% | 0-5% | 0-5% | 0-5% |
| Y | 0-5% | 0-5% | 0-5% | 0-5% |
| Zr | 20-40% | 22-38% | 27-33% | 1-15% |
| Component/Wt. % | Ex. 191 | Ex. 192 | Ex. 193 | Ex. 194 |
| Ag | 0-5% | 0-5% | 0-5% | 0-5% |
| Al | 0-5% | 0-5% | 0-5% | 0-5% |
| B | 0-5% | 0-5% | 0-5% | 0-5% |
| Bi | 0-5% | 0-5% | 0-5% | 0-5% |
| Cr | 0-5% | 0-5% | 0-5% | 0-5% |
| Cu | 0-5% | 0-5% | 0-5% | 0-5% |
| Co | 0-5% | 0-5% | 0-5% | 0-5% |
| Fe | 0-5% | 0-5% | 0-5% | 0-5% |
| Hf | 0-5% | 0-5% | 0-5% | 0-5% |
| Ir | 0-5% | 0-5% | 0-5% | 0-5% |
| La | 0-5% | 0-5% | 0-5% | 0-5% |
| Mg | 0-5% | 0-5% | 0-5% | 0-5% |
| Mn | 0-5% | 0-5% | 0-5% | 0-5% |
| Mo | 0-5% | 0-5% | 0-5% | 0-5% |
| Nb | 25-40% | 30-40% | 25-40% | 26-32% |
| Ni | 0-5% | 0-5% | 0-5% | 0-5% |
| Os | 0-5% | 0-5% | 0-5% | 0-5% |
| Pt | 0-5% | 0-5% | 0-5% | 0-5% |
| Re | 0-5% | 0-5% | 0-5% | 0-5% |
| Rh | 0-5% | 0-5% | 0-5% | 0-5% |
| Se | 0-5% | 0-5% | 0-5% | 0-5% |
| Si | 0-5% | 0-5% | 0-5% | 0-5% |
| Sn | 0-5% | 0-5% | 0-5% | 0-5% |
| Ta | 2-8% | 3-6% | 5-15% | 10-14% |
| Tc | 0-5% | 0-5% | 0-5% | 0-5% |
| Ti | 51-70% | 52-63% | 51-68% | 51-62% |
| V | 0-5% | 0-5% | 0-5% | 0-5% |
| W | 0-5% | 0-5% | 0-5% | 0-5% |
| Y | 0-5% | 0-5% | 0-5% | 0-5% |
| Zr | 2-12% | 4-8% | 2-8% | 2-6% |
| Component/Wt. % | Ex. 195 | Ex. 196 | Ex. 197 | Ex. 198 |
| Ag | 0-5% | 0-5% | 0-5% | 0-5% |
| Al | 0-5% | 0-5% | 0-5% | 0-5% |
| B | 0-5% | 0-5% | 0-5% | 0-5% |
| Bi | 0-5% | 0-5% | 0-5% | 0-5% |
| Cr | 0-5% | 5-35% | 10-30% | 15-25% |
| Cu | 0-5% | 0-5% | 0-5% | 0-5% |
| Co | 0-5% | 20-55% | 25-50% | 35-45% |
| Fe | 0-5% | 3-25% | 0-5% | 0-5% |
| Hf | 0-5% | 0-5% | 0-5% | 0-5% |
| Ir | 0-5% | 0-5% | 0-5% | 0-5% |
| La | 0-5% | 0-5% | 0-5% | 0-5% |
| Mg | 0-5% | 0-5% | 0-5% | 0-5% |
| Mn | 0-5% | 0-5% | 0-5% | 0-5% |
| Mo | 0-5% | 2-15% | 3-12% | 4-9% |
| Nb | 30-40% | 0-5% | 0-5% | 0-5% |
| Ni | 0-5% | 4-23% | 5-20% | 10-18% |
| Os | 0-5% | 0-5% | 0-5% | 0-5% |
| Pt | 0-5% | 0-5% | 0-5% | 0-5% |
| Re | 0-5% | 0-5% | 0-5% | 0-5% |
| Rh | 0-5% | 0-5% | 0-5% | 0-5% |
| Se | 0-5% | 0-5% | 0-5% | 0-5% |
| Si | 0-5% | 0-5% | 0-5% | 0-5% |
| Sn | 0-5% | 0-5% | 0-5% | 0-5% |
| Ta | 1-3% | 0-5% | 0-5% | 0-5% |
| Tc | 0-5% | 0-5% | 0-5% | 0-5% |
| Ti | 51-67% | 0-5% | 0-5% | 0-5% |
| V | 0-5% | 0-5% | 0-5% | 0-5% |
| W | 0-5% | 0-5% | 0-5% | 0-5% |
| Y | 0-5% | 0-5% | 0-5% | 0-5% |
| Zr | 2-5% | 0-5% | 0-5% | 0-5% |

| Component/Wt. % | Ex. 199 | Ex. 200 | Ex. 201 | Ex. 202 |
|---|---|---|---|---|
| Ag | 0-5% | 0-5% | 0-5% | 0-5% |
| Al | 0-5% | 0-5% | 0-5% | 0-5% |
| B | 0-5% | 0-5% | 0-5% | 0-5% |
| Bi | 0-5% | 0-5% | 0-5% | 0-5% |
| Cr | 0-5% | 0-5% | 0-5% | 0-5% |
| Cu | 0-5% | 0-5% | 0-5% | 0-5% |
| Co | 0-5% | 0-5% | 0-5% | 0-5% |
| Fe | 0-5% | 0-5% | 0-5% | 0-5% |
| Hf | 0-5% | 0-5% | 0-5% | 0-5% |
| Ir | 0-5% | 0-5% | 0-5% | 0-5% |
| La | 0-5% | 0-5% | 0-5% | 0-5% |
| Mg | 0-5% | 0-5% | 0-5% | 0-5% |
| Mn | 0-5% | 0-5% | 0-5% | 0-5% |
| Mo | 30-65% | 40-60% | 45-55% | 0-5% |
| Nb | 0-5% | 0-5% | 0-5% | 55-99.75% |
| Ni | 0-5% | 0-5% | 0-5% | 0-5% |
| Os | 0-5% | 0-5% | 0-5% | 0-5% |
| Pt | 0-5% | 0-5% | 0-5% | 0-5% |
| Re | 0-5% | 0-5% | 0-5% | 0-5% |
| Rh | 0-5% | 0-5% | 0-5% | 0-5% |
| Se | 0-5% | 0-5% | 0-5% | 0-5% |
| Si | 0-5% | 0-5% | 0-5% | 0-5% |
| Sn | 0-5% | 0-5% | 0-5% | 0-5% |
| Ta | 0-5% | 0-5% | 0-5% | 0-5% |
| Tc | 0-5% | 0-5% | 0-5% | 0-5% |
| Ti | 0-5% | 0-5% | 0-5% | 0-5% |
| V | 0-5% | 0-5% | 0-5% | 0-5% |
| W | 0-5% | 0-5% | 0-5% | 0-5% |
| Y | 0-5% | 0-5% | 0-5% | 0-5% |
| Zr | 30-56% | 40-60% | 45-55% | 0.25-45% |

| Component/Wt. % | Ex. 203 | Ex. 204 | Ex. 205 | Ex. 206 |
|---|---|---|---|---|
| Ag | 0-5% | 0-5% | 0-5% | 0-5% |
| Al | 0-5% | 0-5% | 0-5% | 0-5% |
| B | 0-5% | 0-5% | 0-5% | 0-5% |
| Bi | 0-5% | 0-5% | 0-5% | 0-5% |
| Cr | 0-5% | 0-5% | 0-5% | 0-5% |
| Cu | 0-5% | 0-5% | 0-5% | 0-5% |
| Co | 0-5% | 0-5% | 0-5% | 0-5% |
| Fe | 0-5% | 0-5% | 0-5% | 0-5% |
| Hf | 0-5% | 0-5% | 0-5% | 0-5% |
| Ir | 0-5% | 0-5% | 0-5% | 0-5% |
| La | 0-5% | 0-5% | 0-5% | 0-5% |
| Mg | 0-5% | 0-5% | 0-5% | 0-5% |
| Mn | 0-5% | 0-5% | 0-5% | 0-5% |
| Mo | 0-5% | 0-5% | 0-5% | 0-5% |
| Nb | 75-99.5% | 95-99.25% | 55-78.5% | 68-74.25% |
| Ni | 0-5% | 0-5% | 0-5% | 0-5% |
| Os | 0-5% | 0-5% | 0-5% | 0-5% |
| Pt | 0-5% | 0-5% | 0-5% | 0-5% |
| Re | 0-5% | 0-5% | 0-5% | 0-5% |
| Rh | 0-5% | 0-5% | 0-5% | 0-5% |
| Se | 0-5% | 0-5% | 0-5% | 0-5% |
| Si | 0-5% | 0-5% | 0-5% | 0-5% |
| Sn | 0-5% | 0-5% | 0-5% | 0-5% |
| Ta | 0-5% | 0-5% | 20-35% | 25-30% |
| Tc | 0-5% | 0-5% | 0-5% | 0-5% |
| Ti | 0-5% | 0-5% | 0-5% | 0-5% |
| V | 0-5% | 0-5% | 0-5% | 0-5% |
| W | 0-5% | 0-5% | 1-8% | 0-5% |
| Y | 0-5% | 0-5% | 0-5% | 0-5% |
| Zr | 0.5-25% | 0.75-5% | 0.5-5% | 0.75-3% |

| Element/Wt. % | Ex. 207 | Ex. 208 | Ex. 209 | Ex. 210 |
|---|---|---|---|---|
| Re | 30-75% | 40-75% | 45-75% | 45-70% |
| Cr | 25-70% | 25-65% | 25-55% | 30-55% |
| Mo | 0-25% | 0-25% | 1-25% | 2-25% |
| Bi | 0-25% | 0-25% | 0-25% | 0-25% |
| Cr | 0-25% | 0-25% | 0-25% | 0-25% |
| Ir | 0-25% | 0-25% | 0-25% | 0-25% |
| Nb | 0-25% | 0-25% | 0-25% | 0-25% |
| Ta | 0-25% | 0-25% | 0-25% | 0-25% |
| V | 0-25% | 0-25% | 0-25% | 0-25% |
| W | 0-25% | 0-25% | 0-25% | 0-25% |
| Mn | 0-25% | 0-25% | 0-25% | 0-25% |
| Tc | 0-25% | 0-25% | 0-25% | 0-25% |
| Ru | 0-25% | 0-25% | 0-25% | 0-25% |
| Rh | 0-25% | 0-25% | 0-25% | 0-25% |
| Hf | 0-25% | 0-25% | 0-25% | 0-25% |
| Os | 0-25% | 0-25% | 0-25% | 0-25% |
| Cu | 0-25% | 0-25% | 0-25% | 0-25% |
| Ir | 0-25% | 0-25% | 0-25% | 0-25% |
| Ti | 0-25% | 0-25% | 0-25% | 0-25% |
| Y | 0-25% | 0-25% | 0-25% | 0-25% |
| Zr | 0-25% | 0-25% | 0-25% | 0-25% |
| C | <0.06% | <0.06% | <0.06% | <0.06% |
| N | <0.06% | <0.06% | <0.06% | <0.06% |
| O | <0.06% | <0.06% | <0.06% | <0.06% |

In Examples 1-210, it will be appreciated that all of the above ranges include any value between the range and any other range that is between the ranges set forth above. Any of the above values that include the <symbol includes the range from 0 to the stated value and all values and ranges therebetween.

In accordance with another and/or alternative non-limiting aspect of the present disclosure, the metal alloy that is used to partially or fully formed the medical device includes rhenium in a sufficient quantity as to create a “rhenium effect” in the metal alloy. As defined herein, a “rhenium effect” is a) an increase of at least 10% in ductility of the metal alloy caused by the addition of rhenium to the metal alloy, and/or b) an increase of at least 10% in tensile strength of the metal alloy caused by the addition of rhenium to the metal alloy. It has been found for many metal alloys results in improved ductility and/or tensile strength. It has been found that the addition of rhenium to a metal alloy can result in the formation of a twining alloy in the metal alloy that results in the overall ductility of the metal alloy to increase as the yield and tensile strength increases as a result of reduction and/or work hardening of the metal alloy that includes the rhenium addition. The “rhenium effect” occurs when the atomic weight of rhenium in the metal alloy is at least 15% (e.g., 15 awt. % to 99 awt. % rhenium in the metal alloy and all values and ranges therebetween). For example, for standard stainless-steel alloys, the “rhenium effect” can begin to be present when the stainless-steel alloy is modified to include a rhenium amount of at least 5-10 wt. % (and all values and ranges therebetween) of the stainless-steel alloy. For standard CoCr alloys, the “rhenium effect” can begin to be present when the CoCr alloy is modified to include a rhenium amount of at least 4.8-9.5 wt. % (and all values and ranges therebetween) of the CoCr alloy. For standard TiAlV alloys, the “rhenium effect” can begin to be present when the TiAlV alloy is modified to include a rhenium amount of at least 4.5-9 wt. % (and all values and ranges therebetween) of the TiAlV alloy. At can be appreciated, the rhenium content in the above examples can be greater than the minimum amount to create the “rhenium effect” in the metal alloy.

In accordance with another and/or alternative aspect of the present disclosure, the metal alloy includes at least 15 awt. % (e.g., 10-99 awt. % and all values and ranges therebetween) of the metal alloy includes rhenium. In one non-limiting embodiment, the metal alloy includes at least 15 awt. % (e.g., 15-99.9 awt. % and all values and ranges therebetween) rhenium, and 0.1-95.5 wt. % (and all values and ranges therebetween) of one or more additives selected from the group of aluminum, boron, beryllium, bismuth, cadmium, calcium, cerium, cerium oxide, chromium, cobalt, copper, gallium, gold, hafnium, iridium, iron, lanthanum, lanthanum oxide, lead, lithium, magnesium, manganese, molybdenum, nickel, niobium, osmium, palladium, platinum, rare earth metals, rhodium, ruthenium, scandium, silver, silicon, tantalum, technetium, tin, titanium, tungsten, vanadium, yttrium, yttrium oxide, zinc, zirconium, and/or zirconium oxide. In another non-limiting embodiment, the metal alloy includes at least 20 awt. % (e.g., 20-99.9 awt. % and all values and ranges therebetween) rhenium, and 0.1-94 wt. % (and all values and ranges therebetween) of one or more additives selected from the group of aluminum, boron, beryllium, bismuth, cadmium, calcium, cerium, cerium oxide, chromium, cobalt, copper, gallium, gold, hafnium, iridium, iron, lanthanum, lanthanum oxide, lead, lithium, magnesium, manganese, molybdenum, nickel, niobium, osmium, palladium, platinum, rare earth metals, rhodium, ruthenium, scandium, silver, silicon, tantalum, technetium, tin, titanium, tungsten, vanadium, yttrium, yttrium oxide, zinc, zirconium, and/or zirconium oxide, and the metal alloy includes 0-2 wt. % (and all values and ranges therebetween) of a combination of other metals, carbon, oxygen, phosphorous, sulfur, hydrogen and nitrogen.

In accordance with another and/or alternative aspect of the present disclosure, the metal alloy includes 35-75 wt. % (e.g., and all values and ranges therebetween) of the metal alloy includes rhenium, and 25-65 wt. % (and all values and ranges therebetween) of the metal alloy includes two or more of aluminum, boron, beryllium, bismuth, cadmium, calcium, cerium, cerium oxide, chromium, cobalt, copper, gallium, gold, hafnium, iridium, iron, lanthanum, lanthanum oxide, lead, lithium, magnesium, manganese, molybdenum, nickel, niobium, osmium, palladium, platinum, rare earth metals, rhodium, ruthenium, scandium, silver, silicon, tantalum, technetium, tin, titanium, tungsten, vanadium, yttrium, yttrium oxide, zinc, zirconium, and/or zirconium oxide, and the metal alloy includes 0-2 wt. % of a combination of other metals, carbon, oxygen, phosphorous, sulfur, hydrogen and nitrogen. In one non-limiting embodiment, the metal alloy includes 50-75 wt. % rhenium, 24-49 wt. % chromium, 1-15 wt. % molybdenum, and 0-25 wt. % one or more of aluminum, boron, beryllium, bismuth, cadmium, calcium, cerium, cerium oxide, cobalt, copper, gallium, gold, hafnium, iridium, iron, lanthanum, lanthanum oxide, lead, lithium, magnesium, manganese, nickel, niobium, osmium, palladium, platinum, rare earth metals, rhodium, ruthenium, scandium, silver, silicon, tantalum, technetium, tin, titanium, tungsten, vanadium, yttrium, yttrium oxide, zinc, zirconium, and/or zirconium oxide, and the metal alloy includes 0-2 wt. % of a combination of other metals, carbon, oxygen, phosphorous, sulfur, hydrogen and nitrogen.

In accordance with another and/or alternative non-limiting aspect of the present disclosure, the metal alloy that is used to partially or fully formed the medical device includes at least 15 awt. % rhenium (e.g., 15-99.9 awt. % and all values and ranges therebetween), and at least 0.1 wt. % (e.g., 0.1 wt. % to 96 wt. % an all values and ranges therebetween) of one or more additives selected from the group of aluminum, bismuth, calcium, carbon, cerium oxide, chromium, cobalt, copper, gold, hafnium, iridium, iron, lanthanum, lanthanum oxide, lead, magnesium, manganese, molybdenum, nickel, niobium, osmium, platinum, rare earth metals, rhodium, ruthenium, silver, tantalum, technetium, titanium, tungsten, vanadium, yttrium, yttrium oxide, zinc, zirconium, and/or zirconium oxide. In another non-limiting embodiment, the metal alloy includes at least 15 awt. % rhenium (e.g., 15-99.9 awt. % and all values and ranges therebetween), and at least 0.1 wt. % (e.g., 0.1 wt. % to 96 wt. % an all values and ranges therebetween) of two or more additives selected from the group of aluminum, bismuth, calcium, carbon, cerium oxide, chromium, cobalt, copper, gold, hafnium, iridium, iron, lanthanum, lanthanum oxide, lead, magnesium, manganese, molybdenum, nickel, niobium, osmium, platinum, rare earth metals, rhodium, ruthenium, silver, tantalum, technetium, titanium, tungsten, vanadium, yttrium, yttrium oxide, zinc, zirconium, and/or zirconium oxide. In another non-limiting embodiment, the metal alloy includes at least 15 awt. % rhenium (e.g., 15-99.9 awt. % and all values and ranges therebetween), and at least 0.1 wt. % (e.g., 0.1 wt. % to 96 wt. % an all values and ranges therebetween) of three or more additives selected from the group of aluminum, bismuth, calcium, carbon, cerium oxide, chromium, cobalt, copper, gold, hafnium, iridium, iron, lanthanum, lanthanum oxide, lead, magnesium, manganese, molybdenum, nickel, niobium, osmium, platinum, rare earth metals, rhodium, ruthenium, silver, tantalum, technetium, titanium, tungsten, vanadium, yttrium, yttrium oxide, zinc, zirconium, and/or zirconium oxide.

In accordance with another and/or alternative non-limiting aspect of the present disclosure, the metal alloy that is used to partially or fully formed the medical device includes at least 30 wt. % (e.g., 30-100 wt. % and all values and ranges therebetween) of the metal alloy includes one or more of molybdenum, niobium, rhenium, tantalum, or tungsten. In another non-limiting embodiment, at least 40 wt. % of the metal alloy includes one or more of molybdenum, niobium, rhenium, tantalum, or tungsten. In another non-limiting embodiment, at least 50 wt. % of the metal alloy includes one or more of molybdenum, niobium, rhenium, tantalum, or tungsten.

In another non-limiting embodiment, the metal alloy that is used to partially or fully formed the medical device includes at least 50 wt. % (e.g., 50-100 wt. % and all values and ranges therebetween) of the metal alloy includes one or more of molybdenum, niobium, rhenium, tantalum, titanium, zirconium or tungsten, and 0-40 wt. % (and all values and ranges therebetween) of the metal alloy includes one or more additives selected from the group of aluminum, bismuth, calcium, carbon, cerium oxide, chromium, cobalt, copper, gold, hafnium, iridium, iron, lanthanum, lanthanum oxide, lead, magnesium, manganese, nickel, osmium, platinum, rare earth metals, rhodium, ruthenium, silver, technetium, vanadium, yttrium, yttrium oxide, zinc, and/or zirconium oxide, and the metal alloy includes 0-2 wt. % (and all values and ranges therebetween) of a combination of other metals, carbon, oxygen, phosphorous, sulfur, hydrogen and nitrogen.

In accordance with another and/or alternative non-limiting aspect of the present disclosure, the metal alloy that is used to partially or fully formed the medical device includes at least 20 wt. % (e.g., 20-99 wt. % and all values and ranges therebetween) of the metal alloy includes rhenium. In one non-limiting embodiment, the metal alloy includes at least 20 wt. % (e.g., 20-99.9 wt. % and all values and ranges therebetween) rhenium, and 0.1-80 wt. % (and all values and ranges therebetween) of one or more additives selected from the group of aluminum, bismuth, calcium, carbon, cerium oxide, chromium, cobalt, copper, gold, hafnium, iridium, iron, lanthanum, lanthanum oxide, lead, magnesium, manganese, molybdenum, nickel, niobium, osmium, platinum, rare earth metals, rhodium, ruthenium, silver, tantalum, technetium, titanium, tungsten, vanadium, yttrium, yttrium oxide, zinc, zirconium, and/or zirconium oxide, and the metal alloy includes 0-2 wt. % (and all values and ranges therebetween) of a combination of other metals, carbon, oxygen, phosphorous, sulfur, hydrogen and nitrogen. In another non-limiting embodiment, 35-60 wt. % (e.g., and all values and ranges therebetween) of the metal alloy includes rhenium, and 40-65 wt. % (and all values and ranges therebetween) of the metal alloy includes two or more of molybdenum, niobium, tantalum, tantalum, titanium, vanadium, chromium, manganese, zirconium, technetium, ruthenium, rhodium, hafnium, osmium, copper, and/or iridium. In another non-limiting embodiment, 35-60 wt. % of the metal alloy includes rhenium, and 40-65 wt. % of the metal alloy includes three or more of molybdenum, niobium, tantalum, tantalum, titanium, vanadium, chromium, manganese, zirconium, technetium, ruthenium, rhodium, hafnium, osmium, copper, and/or iridium. In another non-limiting embodiment, a weight percent of molybdenum in the metal alloy is at least 10 wt. % and less than 60 wt. % (and all values and ranges therebetween). In another non-limiting embodiment, a weight percent of rhenium in the metal alloy is 35-60 wt. % (and all values and ranges therebetween). In another non-limiting embodiment, a combined weight percent of the alloying metals is 5-45 wt. % (and all values and ranges therebetween) of the metal alloy. In another non-limiting embodiment, a weight percent of the rhenium in the metal alloy is greater than a combined weight percent of the alloying metals. In another non-limiting embodiment, a combined weight percent of the rhenium, molybdenum, and the one or more alloying metals in the metal alloy is at least 99.9 wt. %. In accordance with another and/or alternative non-limiting aspect of the present disclosure, the atomic weight percent of rhenium to the atomic weight percent of the combination of bismuth, niobium, tantalum, tungsten, titanium, vanadium, chromium, manganese, yttrium, zirconium, technetium, ruthenium, rhodium, hafnium, osmium, copper, and iridium in the metal alloy is 0.7:1 to 1.5:1 (and all values and ranges therebetween), typically 0.8:1 to 1.4:1, more typically 0.8:1 to 1.25:1, and still more typically about 0.9:1 to 1.1:1 (e.g., 1:1). In one specific non-limiting formulation, the atomic weight percent of rhenium to the atomic weight percent of the combination of bismuth, chromium, iridium, niobium, tantalum, titanium, yttrium, and zirconium is 0.7:1 to 5.1:1 (and all values and ranges therebetween), typically 0.8:1 to 1.5:1, more typically 0.8:1 to 1.25:1, and still more typically about 0.9:1 to 1.1:1 (e.g., 1:1). In one specific non-limiting formulation, the atomic weight percent of rhenium to the atomic weight percent of the combination of chromium, niobium, tantalum, and zirconium is 0.7:1 to 5.1:1 (and all values and ranges therebetween), typically 0.8:1 to 1.5:1, more typically 0.8:1 to 1.25:1, and still more typically about 0.9:1 to 1.1:1 (e.g., 1:1). In accordance with another non-limiting embodiment, when the metal alloy includes two of bismuth, niobium, tantalum, tungsten, titanium, vanadium, chromium, manganese, yttrium, zirconium, technetium, ruthenium, rhodium, hafnium, osmium, copper, and iridium, the atomic ratio of the two metals is 0.4:1 to 2.5:1 (and all values and ranges therebetween), and typically 0.5:1 to 2:1.

In another non-limiting aspect of the present disclosure, the metal alloy that is used to partially or fully formed the medical device includes rhenium and tungsten and optionally one or more alloying agents such as, but not limited to, aluminum, bismuth, calcium, carbon, cerium oxide, chromium, cobalt, copper, gold, hafnium, iridium, iron, lanthanum, lanthanum oxide, lead, magnesium, manganese, molybdenum, nickel, niobium, osmium, platinum, rare earth metals, rhodium, ruthenium, silver, tantalum, technetium, titanium, vanadium, yttrium, yttrium oxide, zinc, zirconium, and/or zirconium oxide, and/or alloys of one or more of such components (e.g., WRe, WReMo, etc.), and the metal alloy includes 0-2 wt. % (and all values and ranges therebetween) of a combination of other metals, carbon, oxygen, phosphorous, sulfur, hydrogen and nitrogen. In one non-limiting formulation, the metal alloy includes up to 40 wt. % rhenium and at least 60 wt. % tungsten. In one non-limiting embodiment, the total weight percent of the tungsten and rhenium in the tungsten-rhenium alloy is at least about 95 wt. %, typically at least about 99 wt. %, more typically at least about 99.5 wt. %, yet more typically at least about 99.9 wt. %, and still more typically at least about 99.99 wt. %. In another non-limiting formulation, the metal alloy includes up to 47.5 wt. % rhenium and at least 20-80 wt. % tungsten (and all values and ranges therebetween) and 1-47.5 wt. % molybdenum (and all values and ranges therebetween). In another non-limiting specific tungsten-rhenium-molybdenum alloy, the weight percent of the tungsten is greater than 50 wt. % of the tungsten-rhenium-molybdenum alloy. In another non-limiting specific tungsten-rhenium-molybdenum alloy, the weight percent of the tungsten is greater than a weight percent of rhenium, but less than a weigh percent of molybdenum. In another non-limiting specific tungsten-rhenium-molybdenum alloy, the weight percent of the tungsten is greater than a weight percent of molybdenum, but less than a weigh percent of rhenium. In another non-limiting specific tungsten-rhenium-molybdenum alloy, the weight percent of the tungsten is less than a weight percent of rhenium and also less than the weight percent of molybdenum.

In accordance with another and/or alternative non-limiting aspect of the present disclosure, the metal alloy that is used to partially or fully formed the medical device at least 35 wt. % (e.g., 35-75 wt. % and all values and ranges therebetween) rhenium, and the metal alloy also includes chromium. In one non-limiting embodiment, at least 25 wt. % (e.g., 25-49.9 wt. % and all values and ranges therebetween) of the metal alloy includes chromium. In another non-limiting embodiment, at least 30 wt. % of the metal alloy includes chromium. In another non-limiting embodiment, at least 33 wt. % of the metal alloy includes chromium. In another non-limiting embodiment, at least 50 wt. % (e.g., 50-74.9 wt. % and all values and ranges therebetween) of the metal alloy includes rhenium, at least 25 wt. % (e.g., 25-49.9 wt. % and all values and ranges therebetween) of the metal alloy includes chromium, and 0.1-25 wt. % (and all values and ranges therebetween) of the metal alloy includes one or more of aluminum, bismuth, calcium, carbon, cerium oxide, cobalt, copper, gold, hafnium, iridium, iron, lanthanum, lanthanum oxide, lead, magnesium, manganese, molybdenum, nickel, niobium, osmium, platinum, rare earth metals, rhodium, ruthenium, silver, tantalum, technetium, titanium, tungsten, vanadium, yttrium, yttrium oxide, zinc, zirconium, and/or zirconium oxide. In another non-limiting embodiment, at least 55 wt. % (e.g., 55-69.9 wt. % and all values and ranges therebetween) of the metal alloy includes rhenium, at least 30 wt. % (e.g., 30-44.9 wt. % and all values and ranges therebetween) of the metal alloy includes chromium, and 0.1-15 wt. % (and all values and ranges therebetween) of the metal alloy includes one or more of molybdenum, bismuth, niobium, tantalum, titanium, vanadium, tungsten, manganese, zirconium, technetium, ruthenium, rhodium, hafnium, osmium, copper, yttrium, zirconium, and/or iridium. In another non-limiting embodiment, alloy metal includes chromium. In another non-limiting embodiment, the alloying metal includes chromium and one or more metals selected from the group consisting of bismuth, zirconium, iridium, niobium, tantalum, titanium, and yttrium. In another non-limiting embodiment, the alloying metal includes chromium and one or more metals selected from the group consisting of bismuth, zirconium, iridium, niobium, tantalum, titanium, and yttrium; and wherein an atomic ratio of chromium to an atomic ratio of each or all of the metals selected from the group consisting of bismuth, chromium, iridium, niobium, tantalum, titanium, and yttrium is 0.4:1 to 2.5:1 (and all values and ranges therebetween). In another non-limiting embodiment, the alloying metal includes chromium and one or more metals selected from the group consisting of zirconium, niobium, and tantalum. In another non-limiting embodiment, the alloying metal includes a first metal selected from the group consisting of bismuth, chromium, iridium, niobium, tantalum, titanium, yttrium and zirconium, and a second metal selected from the group consisting of bismuth, chromium, iridium, niobium, tantalum, titanium, yttrium and zirconium; and wherein the first and second metals are different; and wherein an atomic ratio of the first metal to the second metal is 0.4:1 to 2.5:1 (and all values and ranges therebetween). In another non-limiting embodiment, the alloying metal a first metal selected from the group consisting of chromium, niobium, tantalum, and zirconium, and a second metal selected from the group consisting of chromium, niobium, tantalum, and zirconium; and wherein the first and second metals are different; and wherein an atomic ratio of the first metal to the second metal is 0.4:1 to 2.5:1 (and all values and ranges therebetween).

In accordance with another and/or alternative non-limiting aspect of the present disclosure, the metal alloy that is used to partially or fully formed the medical device includes 10-60 atomic weight percent (awt. %) Re (and all values and ranges therebetween) and one or more metals selected from the group consisting of Mo, Cr, Ta, Nb, Ti, and Zr. In one non-limiting embodiment, the metal alloy includes 15-60 awt. % Re and one or more metals selected from the group consisting of Cr, Ta, Nb, Ti, and Zr. In another non-limiting embodiment, the metal alloy includes 15-60 awt. % Re and one or more metals selected from the group consisting of 0.5-70 awt. % Cr (and all values and ranges therebetween), 0.5-70 awt. % Ta (and all values and ranges therebetween), 0.5-70 awt. % Nb (and all values and ranges therebetween), 0.5-70 awt. % Ti (and all values and ranges therebetween), and 0.5-70 awt. % Zr (and all values and ranges therebetween).

In accordance with another and/or alternative non-limiting aspect of the present disclosure, the metal alloy that is used to partially or fully formed the medical device includes 0.5-50 awt. % Re (and all values and ranges therebetween) and 0.5-70 awt. % Cr (and all values and ranges therebetween).

In accordance with another and/or alternative non-limiting aspect of the present disclosure, the metal alloy that is used to partially or fully formed the medical device includes 0.5-50 awt. % Re (and all values and ranges therebetween) and 0.5-70 awt. % Ta (and all values and ranges therebetween).

In accordance with another and/or alternative non-limiting aspect of the present disclosure, the metal alloy that is used to partially or fully formed the medical device includes 0.5-50 awt. % Re (and all values and ranges therebetween) and 0.5-70 awt. % Nb (and all values and ranges therebetween).

In accordance with another and/or alternative non-limiting aspect of the present disclosure, the metal alloy that is used to partially or fully formed the medical device includes 0.5-50 awt. % Re (and all values and ranges therebetween) and 0.5-70 awt. % Ti (and all values and ranges therebetween).

In accordance with another and/or alternative non-limiting aspect of the present disclosure, the metal alloy that is used to partially or fully formed the medical device includes greater than 50 wt. % titanium (e.g., 51-80 wt. % and all values and ranges therebetween), 15-45 wt. % (and all values and ranges therebetween) niobium, 1-10 wt. % (and all values and ranges therebetween) zirconium, and 1-15 wt. % (and all values and ranges therebetween) tantalum. In one non-limiting formulation, the metal alloy includes 58-70 wt. % titanium, 27-37 wt. % niobium, and 2-9 wt. % zirconium, and 1-15 wt. % tantalum.

In accordance with another and/or alternative non-limiting aspect of the present disclosure, the metal alloy that is used to partially or fully formed the medical device includes greater than 50 wt. % titanium (e.g., 51-80 wt. % and all values and ranges therebetween), 15-45 wt. % (and all values and ranges therebetween) niobium, and 1-10 wt. % (and all values and ranges therebetween) molybdenum. In one non-limiting formulation, the metal alloy includes 58-69 wt. % titanium, 27-33 wt. % niobium, and 4-8 wt. % molybdenum.

In accordance with another and/or alternative non-limiting aspect of the present disclosure, the metal alloy that is used to partially or fully formed the medical device includes 30-60 wt. % cobalt (and all values and ranges therebetween), 10-30 wt. % chromium (and all values and ranges therebetween), 5-20 wt. % iron (and all values and ranges therebetween), 5-22 wt. % nickel (and all values and ranges therebetween), and 2-12 wt. % molybdenum (and all values and ranges therebetween). In one non-limiting formulation, the metal alloy includes 35-45 wt. % cobalt, 15-25 wt. % chromium, 12-20 wt. % iron, 10-20 wt. % nickel, and 5-9 wt. % molybdenum.

In accordance with another and/or alternative non-limiting aspect of the present disclosure, the metal alloy that is used to partially or fully formed the medical device includes 40-60 wt. % zirconium (and all values and ranges therebetween), and 40-60 wt. % molybdenum (and all values and ranges therebetween). In one non-limiting formulation, the metal alloy includes 45-55 wt. % cobalt, and 45-55 wt. % molybdenum.

In accordance with another and/or alternative non-limiting aspect of the present disclosure, the metal alloy that is used to partially or fully formed the medical device includes 90-99.5 wt. % niobium (and all values and ranges therebetween), and 0.5-10 wt. % zirconium (and all values and ranges therebetween). In one non-limiting formulation, the metal alloy includes 95-99.25 wt. % niobium, and 0.75-4 wt. % niobium.

In accordance with another and/or alternative non-limiting aspect of the present disclosure, the metal alloy that is used to partially or fully formed the medical device includes 55-75 wt. % niobium (and all values and ranges therebetween), 18-40 wt. % tantalum (and all values and ranges therebetween), 1-7 wt. % tungsten (and all values and ranges therebetween), and 0.5-4 wt. % zirconium (and all values and ranges therebetween). In one non-limiting formulation, the metal alloy includes 60-70 wt. % niobium, 24-32 wt. % tantalum, 2-5 wt. % tungsten, and 0.75-3 wt. % zirconium.

In another and/or alternative non-limiting aspect of the present disclosure, the metal alloy that is used to partially or fully formed the medical device includes rhenium plus at least two metals selected from the group of molybdenum, bismuth, niobium, tantalum, tungsten, titanium, vanadium, chromium, manganese, yttrium, zirconium, technetium, ruthenium, rhodium, hafnium, osmium, copper, and iridium, and the content of metal alloy that includes other elements and compounds is 0-0.1 wt. %, typically 0-0.01 wt. %, and more typically 0-0.001 wt. %. In another specific non-limiting formulation, the metal alloy is formed of rhenium plus at least two metals selected from the group of molybdenum, bismuth, chromium, iridium, niobium, tantalum, titanium, yttrium, and zirconium, and the content of metal alloy that includes other elements and compounds is 0-0.1 wt. %, typically 0-0.01 wt. %, and more typically 0-0.001 wt. %. In another specific non-limiting formulation, the metal alloy is formed of rhenium plus at least three metals selected from the group of rhenium, molybdenum, chromium, niobium, tantalum, and zirconium, and the content of metal alloy that includes other elements and compounds is 0-0.1 wt. %, typically 0-0.01 wt. %, and more typically 0-0.001 wt. %.

In accordance with another and/or alternative non-limiting aspect of the present disclosure, metal alloy includes 10-60 atomic weight percent (awt. %) Re (and all values and ranges therebetween) and one or more metals selected from the group consisting of Mo, Cr, Ta, Nb, Ti, and Zr. In one non-limiting embodiment, the metal alloy includes 15-60 awt. % Re and one or more metals selected from the group consisting of Cr, Ta, Nb, Ti, and Zr. In another non-limiting embodiment, the metal alloy includes 15-60 awt. % Re and one or more metals selected from the group consisting of 0.5-70 awt. % Cr (and all values and ranges therebetween), 0.5-70 awt. % Ta (and all values and ranges therebetween), 0.5-70 awt. % Nb (and all values and ranges therebetween), 0.5-70 awt. % Ti (and all values and ranges therebetween), and 0.5-70 awt. % Zr (and all values and ranges therebetween).

In accordance with another and/or alternative non-limiting aspect of the present disclosure, the metal alloy includes 0.5-50 awt. % Re (and all values and ranges therebetween) and 0.5-70 awt. % Cr (and all values and ranges therebetween).

In accordance with another and/or alternative non-limiting aspect of the present disclosure, the metal alloy includes 0.5-50 awt. % Re (and all values and ranges therebetween) and 0.5-70 awt. % Ta (and all values and ranges therebetween).

In accordance with another and/or alternative non-limiting aspect of the present disclosure, the metal alloy includes 0.5-50 awt. % Re (and all values and ranges therebetween) and 0.5-70 awt. % Nb (and all values and ranges therebetween).

In accordance with another and/or alternative non-limiting aspect of the present disclosure, the metal alloy includes 0.5-50 awt. % Re (and all values and ranges therebetween) and 0.5-70 awt. % Ti (and all values and ranges therebetween).

In accordance with another and/or alternative non-limiting aspect of the present disclosure, the metal alloy includes greater than 50 wt. % titanium (e.g., 51-80 wt. % and all values and ranges therebetween), 15-45 wt. % (and all values and ranges therebetween) niobium, 1-10 wt. % (and all values and ranges therebetween) zirconium, and 1-15 wt. % (and all values and ranges therebetween) tantalum. In one non-limiting formulation, the metal alloy includes 58-70 wt. % titanium, 27-37 wt. % niobium, and 2-9 wt. % zirconium, and 1-15 wt. % tantalum.

In accordance with another and/or alternative non-limiting aspect of the present disclosure, the metal alloy includes greater than 50 wt. % titanium (e.g., 51-80 wt. % and all values and ranges therebetween), 15-45 wt. % (and all values and ranges therebetween) niobium, and 1-10 wt. % (and all values and ranges therebetween) molybdenum. In one non-limiting formulation, the metal alloy includes 58-69 wt. % titanium, 27-33 wt. % niobium, and 4-8 wt. % molybdenum.

In accordance with another and/or alternative non-limiting aspect of the present disclosure, the metal alloy includes 30-60 wt. % cobalt (and all values and ranges therebetween), 10-30 wt. % chromium (and all values and ranges therebetween), 5-20 wt. % iron (and all values and ranges therebetween), 5-22 wt. % nickel (and all values and ranges therebetween), and 2-12 wt. % molybdenum (and all values and ranges therebetween). In one non-limiting formulation, the metal alloy includes 35-45 wt. % cobalt, 15-25 wt. % chromium, 12-20 wt. % iron, 10-20 wt. % nickel, and 5-9 wt. % molybdenum.

In accordance with another and/or alternative non-limiting aspect of the present disclosure, the metal alloy includes 40-60 wt. % zirconium (and all values and ranges therebetween), and 40-60 wt. % molybdenum (and all values and ranges therebetween). In one non-limiting formulation, the metal alloy includes 45-55 wt. % cobalt, and 45-55 wt. % molybdenum.

In accordance with another and/or alternative non-limiting aspect of the present disclosure, the metal alloy includes 90-99.5 wt. % niobium (and all values and ranges therebetween), and 0.5-10 wt. % zirconium (and all values and ranges therebetween). In one non-limiting formulation, the metal alloy includes 95-99.25 wt. % niobium, and 0.75-4 wt. % niobium.

In accordance with another and/or alternative non-limiting aspect of the present disclosure, the metal alloy includes 55-75 wt. % niobium (and all values and ranges therebetween), 18-40 wt. % tantalum (and all values and ranges therebetween), 1-7 wt. % tungsten (and all values and ranges therebetween), and 0.5-4 wt. % zirconium (and all values and ranges therebetween). In one non-limiting formulation, the metal alloy includes 60-70 wt. % niobium, 24-32 wt. % tantalum, 2-5 wt. % tungsten, and 0.75-3 wt. % zirconium.

In another and/or alternative non-limiting aspect of the present disclosure, the metal alloy that is used to partially or fully formed the medical device includes less than about 5 wt. % (e.g., 0-4.999999 wt. % and all values and ranges therebetween) other metals and/or impurities, typically 0-1 wt. %, more typically 0-0.1 wt. %, even more typically 0-0.01 wt. %, and still even more typically 0-0.001 wt. %. A high purity level of the metal alloy results in the formation of a more homogeneous alloy, which in turn results in a more uniform density throughout the metal alloy, and also results in the desired yield and ultimate tensile strengths of the metal alloy.

In another and/or alternative non-limiting aspect of the present disclosure, the metal alloy that is used to partially or fully formed the medical device includes a certain amount of carbon and oxygen; however, this is not required. These two elements have been found to affect the forming properties and brittleness of the metal alloy. The controlled atomic ratio of carbon and oxygen of the metal alloy also can be used to minimize the tendency of the metal alloy to form micro-cracks during the forming of the metal alloy, and/or during the use and/or expansion of the medical device. The carbon to oxygen atomic ratio can be as low as about 0.2:1 (e.g., 0.2:1 to 50:1 and all values and ranges therebetween). Typically, the carbon content of the metal alloy is less than about 0.2 wt. % (e.g., 0 wt. % to 0.1999999 wt. % and all values and ranges therebetween). Carbon contents that are too large can adversely affect the physical properties of the metal alloy. Generally, the oxygen content is to be maintained at very low level. In one non-limiting formulation of the metal alloy, the oxygen content is less than about 0.1 wt. % of the metal alloy (e.g., 0 wt. to 0.0999999 wt. % and all values and ranges therebetween). In another non-limiting embodiment, metal alloy optionally includes a controlled amount of nitrogen; however, this is not required. Large amounts of nitrogen in the metal alloy can adversely affect the ductility of the metal alloy. This can in turn adversely affect the elongation properties of the metal alloy. A too high nitrogen content in the metal alloy can begin to cause the ductility of the metal alloy to unacceptably decrease, thus adversely affect one or more physical properties of the metal alloy that are useful or desired in forming and/or using the medical device. In one non-limiting formulation, the metal alloy includes less than about 0.001 wt. % nitrogen (e.g., 0 wt. % to 0.0009999 wt. % and all values and ranges therebetween). It is believed that the nitrogen content should be less than the content of carbon or oxygen in the metal alloy. In one non-limiting formulation of the metal alloy, the atomic ratio of carbon to nitrogen is at least about 1.5:1 (e.g., 1.5:1 to 400:1 and all values and ranges therebetween). In another non-limiting formulation of the metal alloy, the atomic ratio of oxygen to nitrogen is at least about 1.2:1 (e.g., 1.2:1 to 150:1 and all value and ranges therebetween).

In accordance with another and/or alternative non-limiting aspect of the present disclosure, the metal alloy that is used to partially or fully formed the medical device a) has an average Vickers hardness of optionally at least about 150 Vickers (e.g., 150-300 Vickers and all values and ranges therebetween), b) an average hardness that is greater than stainless steel (e.g., Grade 304, Grade 316), c) an average ultimate tensile strength of optionally at least about 100 ksi (e.g., 100-350 ksi and all values and ranges therebetween), d) an average yield strength of optionally at least about 80 ksi (e.g., 80-300 ksi and all values and ranges therebetween), e) an average grain size of no greater than about 4 ASTM (e.g., 4 ASTM to 20 ASTM using ASTM E112 and all values and ranges therebetween, e.g., 0.35 micron to 90 micron, and all values and ranges therebetween), e) an average tensile elongation of optionally at least about 25% (e.g., 25%-50% average tensile elongation and all values and ranges therebetween).

In accordance with another and/or alternative non-limiting aspect of the present disclosure, there is provided a medical device partially or fully formed of a refractory metal alloy. Non-limiting refractory metal alloys include MoRe alloy, ReW alloy, MoReCr alloy, MoReTa alloy, MoReTi alloy, WCu alloy, ReCr alloy, Mo alloy, Re alloy, W alloy, Ta alloy, Nb alloy, etc. In one non-limiting embodiment, 50-100% (and all values and ranges therebetween) of the medical device is formed of the refractory metal alloy. In another non-limiting embodiment, 50-100% (and all values and ranges therebetween) of the medical device is formed of a MoRe alloy. In another non-limiting embodiment, at least 30 wt. % (e.g., 30-100 wt. % and all values and ranges therebetween) of the refractory metal alloy includes one or more of molybdenum, rhenium, niobium, tantalum or tungsten.

In accordance with another and/or alternative non-limiting aspect of the present disclosure, the refractory metal alloy or the metal alloy that includes at least 15 awt. % rhenium that is used to form at least a portion of the medical device has one or more improved properties (e.g., strength, durability, hardness, biostability, bendability, coefficient of friction, radial strength, flexibility or bendability, tensile strength, tensile elongation, longitudinal lengthening, stress-strain properties, reduced recoil, radiopacity, heat sensitivity, biocompatibility, improved fatigue life, crack resistance, crack propagation resistance, reduced magnetic susceptibility, etc.), improved conformity when bent, less recoil, increase yield strength, improved fatigue ductility, improved durability, improved fatigue life, reduced adverse tissue reactions, reduced metal ion release, reduced corrosion, reduced allergic reaction, improved hydrophilicity, reduced toxicity, reduced thickness of metal component, improved bone fusion, and/or lower ion release into tissue. These one or more improved physical properties of the refractory metal alloy or the metal alloy that includes at least 15 awt. % rhenium can be achieved in the medical device or portion of the medical device (e.g., frame of the medical device, etc.) without having to increase the bulk, volume, and/or weight of the medical device or portion of the medical device (e.g., frame of the medical device, etc.), and in some instances these improved physical properties can be obtained even when the volume, bulk, and/or weight of the medical device or portion of the medical device (e.g., frame of the medical device, etc.) is reduced as compared to medical devices or the frame of the medical device that are at least partially formed from traditional stainless steel, titanium alloy, or cobalt and chromium alloy materials. The reduced amount of recoil, improved bending conformity and greater radial strength of expanded frames that are at least partially formed of a refractory metal alloy or the metal alloy that includes at least 15 awt. % rhenium as compared to expandable frames formed of stainless steel, CoCr alloy, and TiAlV alloy results in one or more of the following non-limiting advantages: 1) formation of a frame for a medical device having thinner posts, struts, and/or strut joints which results in i) safer vascular access when inserting the medical device through a body passageway and to the treatment area, and/or ii) decreased the risk of bleeding and/or damage to the body passageway and/or the treatment area when the medical device is delivered to the treatment area and/or expanded at the treatment area; 2) easier deliverability of the medical device to the treatment area which can result in i) decreased trauma to the body passageway (e.g., blood vessel, aortic arch trauma, etc.) during the insertion and/or expansion of the medical device at the treatment area, and/or ii) decreased risk of neuro complications-stroke; 3) less recoil which results in i) reduced crimping profile size, ii) increased conformability of the expanded medical device at the treatment area after expansion in the treatment area, iii) increased radial strength of the frame of the medical device after expansion at the treatment area, iv) only require a single crimping cycle to crimp the medical device on a balloon catheter or other type of delivery device, v) reduced incidence of damage to components of the medical device (e.g., struts, posts, strut joints, and/or other components of the expandable frame, leaflets, skirts, coatings, etc.) during the crimping, expansion, and operation of the medical device, vi) greater effective orifice area (EOA) of the medical device after expansion of the medical device, vi) decreased pulmonary valve regurgitation (PVR) after expansion of the medical device in the treatment area, and/or vii) require only a single expansion cycle of the balloon on the balloon catheter or other expansion mechanism to fully expand the medical device; and/or 4) creating a medical device having superior material biologic properties to I) improved tissue adhesion and/or growth on or about medical device, II) reduced adverse tissue reactions with the medical device, III) reduced toxicity of medical device, IV) potentially decreased in-valve thrombosis during the life of the medical device, and/or V) reduced incidence of infection during the life of the medical device.

In accordance with another and/or alternative non-limiting aspect of the present disclosure, the refractory metal alloy or the metal alloy that includes at least 15 awt. % rhenium used to at least partially form the medical device or portion of the medical device (e.g., frame of the medical device, etc.) can thus 1) increase the radiopacity of the medical device or portion of the medical device (e.g., frame of the medical device, etc.), 2) increase the radial strength of the medical device or portion of the medical device (e.g., frame of the medical device, etc.), 3) increase the yield strength and/or ultimate tensile strength of the medical device or portion of the medical device (e.g., frame of the medical device, etc.), 4) improve the stress-strain properties of the medical device or portion of the medical device (e.g., frame of the medical device, etc.), 5) improve the crimping and/or expansion properties of the medical device or portion of the medical device (e.g., frame of the medical device, etc.), 6) improve the bendability and/or flexibility of the medical device or portion of the medical device (e.g., frame of the medical device, etc.), 7) improve the strength and/or durability of the medical device or portion of the medical device (e.g., frame of the medical device, etc.), 8) increase the hardness of the medical device or portion of the medical device (e.g., frame of the medical device, etc.), 9) improve the recoil properties of the medical device or portion of the medical device (e.g., frame of the medical device, etc.), 10) improve the biostability and/or biocompatibility properties of the medical device or portion of the medical device (e.g., frame of the medical device, etc.), 11) increase fatigue resistance of the medical device or portion of the medical device (e.g., frame of the medical device, etc.), 12) resist cracking in the medical device or portion of the medical device (e.g., frame of the medical device, etc.) and resist propagation of cracks, 13) enable smaller, thinner, and/or lighter weight medical device or portion of the medical device (e.g., frame of the medical device, etc.) to be made, 14) reduce the outer diameter of a crimped medical device or portion of the medical device (e.g., frame of the medical device, etc.), 15) improve the conformity of the medical device or portion of the medical device (e.g., frame of the medical device, etc.) to the shape of the treatment area when the medical device or portion of the medical device (e.g., frame of the medical device, etc.) is used and/or expanded in the treatment area, 16) reduce the amount of recoil of the medical device or portion of the medical device (e.g., frame of the medical device, etc.) to the shape of the treatment area when the medical device or portion of the medical device (e.g., frame of the medical device, etc.) is expanded in the treatment area, 17) increase yield strength of the medical device or portion of the medical device (e.g., frame of the medical device, etc.), 18) improve fatigue ductility of the medical device or portion of the medical device (e.g., frame of the medical device, etc.), 18) improve durability of the medical device or portion of the medical device (e.g., frame of the medical device, etc.), 19) improve fatigue life of the medical device or portion of the medical device (e.g., frame of the medical device, etc.), 20) reduce adverse tissue reactions after implant of the medical device or portion of the medical device (e.g., frame of the medical device, etc.), 21) reduce metal ion release after implant of the medical device or portion of the medical device (e.g., frame of the medical device, etc.), 22) reduce corrosion of the medical device or portion of the medical device (e.g., frame of the medical device, etc.) after implant of the medical device or portion of the medical device (e.g., frame of the medical device, etc.), 23) reduce allergic reaction after implant of the medical device or portion of the medical device (e.g., frame of the medical device, etc.), 24) improve hydrophilicity of the medical device or portion of the medical device (e.g., frame of the medical device, etc.), 25) reduce thickness of meta component of medical device or portion of the medical device (e.g., frame of the medical device, etc.), 26) improve bone fusion with medical device or portion of the medical device (e.g., frame of the medical device, etc.), and/or 27) lower ion release from medical device or portion of the medical device (e.g., frame of the medical device, etc.) into tissue, 28) reduce magnetic susceptibility of the medical device or portion of the medical device (e.g., frame of the medical device, etc.) when implanted in a patient, and/or 29) reduce toxicity of the medical device or portion of the medical device (e.g., frame of the medical device, etc.) after implant of the prosthetic medical device.

In accordance with another and/or alternative non-limiting aspect of the present disclosure, the medical device or portion of the medical device (e.g., frame of the medical device, etc.) is optionally subjected to one or more manufacturing processes. These manufacturing processes can include, but are not limited to, expansion, laser cutting, shaving, plug drawing, etching (chemical etching, plasma etching, etc.), crimping, photo-etching, coating, annealing, centerless grinding, turning, drawing, pilgering, electroplating, polishing, electro-polishing, machining, plasma coating, 3D printing, 3D printed coatings, cold working, drilling (e.g., gun drilling, etc.), chemical vapor deposition, chemical polishing, cleaning, buffing, smoothing, pickling, ion beam deposition or implantation, sputter coating, vacuum deposition, swaging, nitriding, annealing, EDM cutting, microelectromechanical manufacturing (MEMS) techniques [e.g., micro-machining, laser micro-machining, laser micro-machining, micro-molding, etc.], etc. In one non-limiting embodiment, the medical device is optionally subjected to a swaging process. The swaging operation can be performed on the medical device in the areas to be hardened. In one non-limiting arrangement, the swaging temperature can be from room temperature (RT) (e.g., 10-27° C. and all values and ranges therebetween) to about 400° C. (e.g., 10-400° C. and all values and ranges therebetween) if the swaging is conducted in air or an oxidizing environment. The swaging temperature can be increased to up to about 1500° C. (e.g., 10-1500° C. and all values and ranges therebetween) if the swaging process is performed in a controlled neutral or non-reducing environment (e.g., inert environment). During the swaging process ions, of boron and/or nitrogen can optionally impinge upon rhenium atoms in the metal alloys that include rhenium to form ReB₂, ReN₂ and/or ReN₃. In another non-limiting embodiment, the medical device is optionally subjected to nitriding (e.g., gas nitriding, salt bath nitriding, plasma nitriding, etc.). The thickness of the nitrided surface layer is less than about 1 mm. In one non-limiting embodiment, the thickness of the nitrided surface layer is at least about 50 nanometer and less than about 1 mm (and all values and ranges therebetween). Generally, the weight percent of nitrogen in the nitrided surface layer is 0.0001-5 wt. % nitrogen (and all values and ranges therebetween). Generally, the weight percent of nitrogen in the nitrided surface layer is generally less than at least one and generally all of the primary components (e.g., primary components are alloy components that constitute more than 5 wt. % of the metal alloy) of the metal alloy. The nitriding process for the metal alloy can be used to increase surface hardness and/or wear resistance metal alloy, increase surfaces slickness or lubricity of the metal alloy and/or to inhibit or prevent discoloration of the metal alloy (e.g., discoloration by oxidation, etc.). In another non-limiting embodiment, the metal alloy can be optionally nitrided prior and/or after to at least one drawing step and/or annealing step for the metal alloy.

In accordance with another and/or alternative non-limiting aspect of the present disclosure, the refractory metal alloy or the metal alloy that includes at least 15 awt. % rhenium that is used to at least partially form the medical device or portion of the medical device (e.g., frame of the medical device, etc.) optionally has a generally uniform density throughout the refractory metal alloy or the metal alloy that includes at least 15 awt. % rhenium, and also results in the desired yield and ultimate tensile strengths of the refractory metal alloy or the metal alloy that includes at least 15 awt. % rhenium. The density of the refractory metal alloy or the metal alloy that includes at least 15 awt. % rhenium is generally at least about 5 gm/cc (e.g., 5 gm/cc-21 gm/cc and all values and ranges therebetween; 10-20 gm/cc; etc.), and typically at least about 11-19 gm/cc. This substantially uniform high density of the refractory metal alloy or the metal alloy that includes at least 15 awt. % rhenium can optionally improve the radiopacity of the refractory metal alloy or the metal alloy that includes at least 15 awt. % rhenium.

In another and/or alternative non-limiting aspect of the present disclosure, the refractory metal alloy or the metal alloy that includes at least 15 awt. % rhenium used to form all or part of the medical device 1) is optionally not clad, metal sprayed, plated, and/or formed (e.g., cold worked, hot worked, etc.) onto another metal, or 2) optionally does not have another metal or metal alloy metal sprayed, plated, clad, and/or formed onto the refractory metal alloy or the metal alloy that includes at least 15 awt. % rhenium.

In accordance with another and/or alternative non-limiting aspect of the present disclosure, the refractory metal alloy or the metal alloy that includes at least 15 awt. % rhenium that is used to form all or part of the medical device 1) is clad, metal sprayed, plated and/or formed (e.g., cold worked, hot worked, etc.) onto another metal, or 2) has another metal or metal alloy metal sprayed, plated, clad and/or formed onto the refractory metal alloy or the metal alloy that includes at least 15 awt. % rhenium.

In accordance with another and/or alternative non-limiting aspect of the present disclosure, the medical device or portion of the medical device (e.g., frame of the medical device, etc.) can optionally be at least partially or fully formed from a tube or rod of the refractory metal alloy or the metal alloy that includes at least 15 awt. % rhenium, or be formed into shape that is at least 80% of the final net shape of the medical device or portion of the medical device (e.g., frame of the medical device, etc.).

In accordance with another and/or alternative non-limiting aspect of the present disclosure, the refractory metal alloy or the metal alloy that includes at least 15 awt. % rhenium has several physical properties that positively affect the medical device when the medical device is at least partially formed of the refractory metal alloy or the metal alloy that includes at least 15 awt. % rhenium. In one non-limiting embodiment of the disclosure, the average Vickers hardness of the refractory metal alloy or the metal alloy that includes at least 15 awt. % rhenium used to at least partially form the medical device or portion of the medical device (e.g., frame of the medical device, etc.) is optionally at least about 150 Vickers (e.g., 150-300 Vickers and all values and ranges therebetween); and typically 160-240 Vickers; however, this is not required. The refractory metal alloy or the metal alloy that includes at least 15 awt. % rhenium generally has an average hardness that is greater than stainless steel (e.g., Grade 304, Grade 316). In another and/or alternative non-limiting embodiment of the disclosure, the average ultimate tensile strength of the refractory metal alloy or the metal alloy that includes at least 15 awt. % rhenium is optionally at least about 100 ksi (e.g., 100-350 ksi and all values and ranges therebetween); however, this is not required. In still another and/or alternative non-limiting embodiment of the disclosure, the average yield strength of the refractory metal alloy or the metal alloy that includes at least 15 awt. % rhenium is optionally at least about 80 ksi (e.g., 80-300 ksi and all values and ranges therebetween); however, this is not required. In yet another and/or alternative non-limiting embodiment of the disclosure, the average grain size of the refractory metal alloy or the metal alloy that includes at least 15 awt. % rhenium used to at least partially form the medical device or portion of the medical device (e.g., frame of the medical device, etc.) is optionally no greater than about 4 ASTM (e.g., 4 ASTM to 20 ASTM using ASTM E112 and all values and ranges therebetween, e.g., 0.35 micron to 90 micron, and all values and ranges therebetween). The small grain size of the refractory metal alloy or the metal alloy that includes at least 15 awt. % rhenium enables the medical device or portion of the medical device (e.g., frame of the medical device, etc.) to have the desired elongation and ductility properties that are useful in enabling the medical device or portion of the medical device (e.g., frame of the medical device, etc.) to be formed, crimped, and/or expanded.

In another and/or alternative non-limiting embodiment of the disclosure, the average tensile elongation of the refractory metal alloy or the metal alloy that includes at least 15 awt. % rhenium used to at least partially form the medical device or portion of the medical device (e.g., frame of the medical device, etc.) is optionally at least about 25% (e.g., 25%-50% average tensile elongation and all values and ranges therebetween). An average tensile elongation of at least 25% for the refractory metal alloy or the metal alloy that includes at least 15 awt. % rhenium is useful to facilitate in the medical device or portion of the medical device (e.g., frame of the medical device, etc.) being properly expanded when positioned in the treatment area of a body passageway. A medical device or frame of a medical device that is partially or fully formed of a material that does not have an average tensile elongation of at least about 25% may be more prone to the formation of micro-cracks and/or break during the forming, crimping, and/or expansion of the medical device or portion of the medical device (e.g., frame of the medical device, etc.).

In accordance with another and/or alternative non-limiting aspect of the present disclosure, the medical device can optionally include, contain and/or be coated with one or more agents that facilitate in the success of the medical device and/or the treatment area. The term “agent” includes, but is not limited to a substance, pharmaceutical, biologic, veterinary product, drug, and analogs or derivatives otherwise formulated and/or designed to prevent, inhibit and/or treat one or more clinical and/or biological events, and/or to promote healing. Non-limiting examples of clinical events that can be addressed by one or more agents include, but are not limited to, viral, fungus and/or bacterial infection; vascular diseases and/or disorders; digestive diseases and/or disorders; reproductive diseases and/or disorders; lymphatic diseases and/or disorders; cancer; implant rejection; pain; nausea; swelling; arthritis; bone diseases and/or disorders; organ failure; immunity diseases and/or disorders; cholesterol problems; blood diseases and/or disorders; lung diseases and/or disorders; heart diseases and/or disorders; brain diseases and/or disorders; neuralgia diseases and/or disorders; kidney diseases and/or disorders; ulcers; liver diseases and/or disorders; intestinal diseases and/or disorders; gallbladder diseases and/or disorders; pancreatic diseases and/or disorders; psychological disorders; respiratory diseases and/or disorders; gland diseases and/or disorders; skin diseases and/or disorders; hearing diseases and/or disorders; oral diseases and/or disorders; nasal diseases and/or disorders; eye diseases and/or disorders; fatigue; genetic diseases and/or disorders; burns; scarring and/or scars; trauma; weight diseases and/or disorders; addiction diseases and/or disorders; hair loss; cramps; muscle spasms; tissue repair; nerve repair; neural regeneration and/or the like. The type and/or amount of agent included in medical device and/or coated on medical device can vary. When two or more agents are included in and/or coated on medical device, the amount of two or more agents can be the same or different. The type and/or amount of agent included on, in and/or in conjunction with medical device are generally selected to address one or more clinical events. The amount of agent included on, in and/or used in conjunction with medical device, when the agent is used, is about 0.01-100 ug per mm2 (and all values and ranges wherein between) and/or at least about 0.00001 wt. % of the medical device; however, other amounts can be used. The amount of two of more agents on, in and/or used in conjunction with medical device can be the same or different. The one or more agents can be coated on and/or impregnated in medical device by a variety of mechanisms such as, but not limited to, spraying (e.g., atomizing spray techniques, etc.), flame spray coating, powder deposition, dip coating, flow coating, dip-spin coating, roll coating (direct and reverse), sonication, brushing, plasma deposition, depositing by vapor deposition, MEMS technology, and rotating mold deposition. The amount of two of more agents on, in and/or used in conjunction with medical device, when two one more agents are used, can be the same or different. The medical device can be configured such that 1) all the agent on and/or in the medical device is controllably released, 2) some of the agent on and/or in the medical device is controllably released and some of the agent on the medical device is non-controllably released, or 3) none of the agent on and/or in the medical device is controllably released. The medical device can also be designed such that the rate of release of the one or more agents from the medical device is the same or different. The medical device can also be designed such that the rate of release of the one or more agents from one or more regions on the medical device is the same or different. Non-limiting arrangements that can be used to control the release of one or more agents from the medical device include 1) at least partially coat one or more agents with one or more polymers, 2) at least partially incorporate and/or at least partially encapsulate one or more agents into and/or with one or more polymers, and/or 3) insert one or more agents in pores, passageway, cavities, etc. in the medical device and at least partially coat or cover such pores, passageway, cavities, etc. with one or more polymers. As can be appreciated, other or additional arrangements can be used to control the release of one or more agents from the medical device. The thickness of each polymer layer and/or layer of agent, when used, is generally at least about 0.01 μm and is generally less than about 150 μm (e.g., 0.01-149.9999 μm and all values and ranges therebetween).

In accordance with another and/or alternative non-limiting aspect of the present disclosure, the medical device can optionally include marker material that facilitates enabling the medical device to be properly positioned in the treatment area. The marker material is typically designed to be visible to electromagnetic waves (e.g., x-rays, microwaves, visible light, infrared waves, ultraviolet waves, etc.); sound waves (e.g., ultrasound waves, etc.); magnetic waves (e.g., MRI, etc.); and/or other types of electromagnetic waves (e.g., microwaves, visible light, infrared waves, ultraviolet waves, etc.). In one non-limiting embodiment, the marker material is visible to x-rays (i.e., radiopaque). The marker material can form all or a portion of the medical device and/or be coated on one or more portions the medical device. The location of the marker material can be on one or multiple locations on the medical device.

In accordance with another and/or alternative non-limiting aspect of the present disclosure, the medical device is expandable device by use of some other device (e.g., balloon, etc.).

In accordance with another and/or alternative non-limiting aspect of the present disclosure, the medical device is fabricated from a material having no or substantially no shape-memory characteristics.

In accordance with another and/or alternative non-limiting aspect of the present disclosure, the net part, blank, rod, tube, etc. that is used to partially or fully form the medical device can be formed by various techniques such as, but not limited to, 1) melting the metal alloy and/or metals that form the metal alloy (e.g., vacuum arc melting, etc.) and then extruding and/or casting the metal alloy into a near net part, blank, rod, tube, etc., 2) melting the metal alloy and/or metals that form the metal alloy, forming a metal strip and then rolling and welding the strip into a near net part, blank, rod, tube, etc., 3) consolidating (pressing, pressing and sintering, etc.) the metal powder of the metal alloy and/or metal powder of metals that form the metal alloy into a near net part, blank, rod, tube, etc., and/or 4) 3D print the metal alloy into a near net part, blank, rod, tube, etc.

In accordance with another and/or alternative non-limiting aspect of the present disclosure, there is optionally provided a near net process for the medical device. In one non-limiting embodiment of the disclosure, there is provided a method of powder pressing materials and optionally increasing the strength post-sintering by imparting additional cold work. In one non-limiting embodiment, the green part is pressed and then sintered. Thereafter, the sintered part is again pressed to increase its mechanical strength by imparting cold work into the pressed and sintered part. Generally, the temperature during the pressing process after the sintering process is 20-100° C. (and all values and ranges therebetween), typically 20-80° C., and more typically 20-40° C. As defined herein, cold working occurs at a temperature of no more than 150° C. (e.g., 10-150° C. and all values and ranges therebetween). The change in the shape of the repressed post-sintered part needs to be determined so the final part (pressed, sintered, and re-pressed) meets the dimensional requirements of the final formed part. A prepress pressure of 1-300 tsi (1 ton per square inch) (and all values and ranges therebetween) can be used followed by a sintering process of at least 1600° C. (e.g., 1600-2600° C. and all values and ranges therebetween) and a post sintering press at a pressure of 1-300 tsi (and all values and ranges therebetween) at a temperature of at least 20° C. (e.g., 20-100° C. and all values and ranges therebetween; 20-40° C., etc.).

In accordance with another and/or alternative non-limiting aspect of the present disclosure, there is optionally provided a press of near net or finished part composite for the medical device that includes the use of a degradable polymer. The process includes pressing a composite structure formed of metal powder and polymer for purposes of making complex part geometries and foam like structures. When the pressed part is sintered, the polymer is partially or fully removed through a process of thermal degradation of the polymer. The resulting part has a porosity associated with the size of the polymer particles as well as the homogeneity of the mixture upon pressing prior to sintering. Generally, the polymer constitutes about 0.1-70 vol. % (and all values and ranges therebetween) of the consolidated and pressed material prior to the sintering step.

In accordance with another and/or alternative non-limiting aspect of the present disclosure, the medical device can optionally include one or more surface structures (e.g., pore, channel, pit, rib, slot, notch, bump, teeth, needle, well, hole, groove, etc.). These structures can be at least partially formed by MEMS (e.g., micro-machining, etc.) technology and/or other types of technology (e.g., 3D printing, etc.). In accordance with one non-limiting embodiment, the medical device can optionally include one or more micro-structures (e.g., micro-needle, micro-pore, micro-cylinder, micro-cone, micro-pyramid, micro-tube, micro-parallelopiped, micro-prism, micro-hemisphere, teeth, rib, ridge, ratchet, hinge, zipper, zip-tie-like structure, etc.) on the surface of the medical device. As defined herein, a “micro-structure” is a structure having at least one dimension (e.g., average width, average diameter, average height, average length, average depth, etc.) that is no more than about 2 mm, and typically no more than about 1 mm. As can be appreciated, when the medical device includes one or more surface structures, 1) all the surface structures can be micro-structures, 2) all the surface structures can be non-micro-structures, or 3) a portion of the surface structures can be micro-structures and a portion can be non-micro-structures. Non-limiting examples of structures that can be formed on the medical device are illustrated in United States Patent Publication Nos. 2004/0093076 and 2004/0093077, which are incorporated herein by reference. Typically, the micro-structures (when formed) extend from or into the outer surface no more than about 400 microns (0.01-400 microns and all values and ranges therebetween), and more typically less than about 300 microns, and more typically about 15-250 microns; however, other sizes can be used. In another one non-limiting embodiment, the one or more surface structures and/or micro-structures can be at least partially formed of an agent and/or be formed of a polymer. One or more of the surface structures and/or micro-structures can include one or more internal passageways that can include one or more materials (e.g., agent, polymer, etc.); however, this is not required.

In accordance with another and/or alternative non-limiting aspect of the present disclosure, the medical device can optionally be an expandable device that can be expanded by use of some other device (e.g., balloon, etc.).

In accordance with another and/or alternative non-limiting aspect of the present disclosure, the medical device can optionally be fabricated from a material having no or substantially no shape-memory characteristics.

In accordance with another and/or alternative non-limiting aspect of the present disclosure, there is optionally provided a near net process for a frame and/or other metal component of the medical device. In one non-limiting embodiment of the disclosure, there is provided a method of powder pressing materials and optionally increasing the strength post-sintering by imparting additional cold work. In one non-limiting embodiment, the green part is pressed and then sintered. Thereafter, the sintered part is again pressed to increase its mechanical strength by imparting cold work into the pressed and sintered part. Generally, the temperature during the pressing process after the sintering process is 20-100° C. (and all values and ranges therebetween), typically 20-80° C., and more typically 20-40° C. As defined herein, cold working occurs at a temperature of no more than 150° C. (e.g., 10-150° C. and all values and ranges therebetween). The change in the shape of the repressed post-sintered part needs to be determined so the final part (pressed, sintered, and re-pressed) meets the dimensional requirements of the final formed part. For a Mo47.5Re alloy, MoRe alloy, ReW alloy, molybdenum alloy, tungsten alloy, rhenium alloy, other refractory metal alloys or other metal alloys that include at least 15 awt. % rhenium, a prepress pressure of 1-300 tsi (1 ton per square inch) (and all values and ranges therebetween) can be used followed by a sintering process of at least 1600° C. (e.g., 1600-2600° C. and all values and ranges therebetween) and a post sintering press at a pressure of 1-300 tsi (and all values and ranges therebetween) at a temperature of at least 20° C. (e.g., 20-100° C. and all values and ranges therebetween; 20-40° C., etc.). There is also provided an optional process of increasing the mechanical strength of a pressed metal part by repressing the post-sintered part to add additional cold work into the material, thereby increasing its mechanical strength. There is also provided an optional process of powder pressing to a near net or final part using metal powder. In one non-limiting embodiment, the metal powder used to form the near net or final part includes a minimum of 40 wt. % rhenium and at least 25 wt. % molybdenum, and remainder can optionally include one or more elements of tungsten, tantalum, chromium, niobium, zirconium, iridium, titanium, bismuth, and yttrium. In another non-limiting embodiment, the metal powder used to form the near net or final part includes 20-80 wt. % rhenium (and all values and ranges therebetween), 20-80 wt. % molybdenum (and all values and ranges therebetween), and optionally one or more elements of tungsten, tantalum, chromium, niobium, zirconium, iridium, titanium, bismuth, and yttrium. In another non-limiting embodiment, the metal powder used to form the near net or final part includes tungsten (20-60 wt. % and all values and ranges therebetween), rhenium (20-80 wt. % and all values and ranges therebetween) and one or more other elements 0-5 wt. % (and all values and ranges therebetween). In another non-limiting embodiment, the metal powder used to form the near net or final part includes tungsten (20-80 wt. % and all values and ranges therebetween), rhenium (20-80 wt. % and all values and ranges therebetween), molybdenum (0.01-15 wt. % and all values and ranges therebetween), and one or more other elements 0-5 wt. % (and all values and ranges therebetween). In another non-limiting embodiment, the metal powder used to form the near net or final part includes tungsten (20-80 wt. % and all values and ranges therebetween), copper (1-30 wt. % and all values and ranges therebetween), and one or more other elements 0-5 wt. % (and all values and ranges therebetween). In another non-limiting embodiment, the metal powder used to form the near net or final part includes 35-65 wt. % rhenium (and all values and ranges therebetween), and two or more elements of tungsten, tantalum, molybdenum, chromium, niobium, zirconium, iridium, titanium, bismuth, and yttrium. In another non-limiting embodiment, the metal powder used to form the near net or final part includes 35-65 wt. % rhenium (and all values and ranges therebetween) molybdenum powder, and 11-41 wt. % (and all values and ranges therebetween) a combination of chromium powder and optionally a powder of one or more metals selected from the group consisting of bismuth, tungsten, tantalum, molybdenum, chromium, niobium, zirconium, iridium, niobium, tantalum, titanium, bismuth, and yttrium. In another non-limiting embodiment, the metal powder used to form the near net or final part includes 35-65 wt. % rhenium (and all values and ranges therebetween), and chromium, and 0.1-25 wt. % (and all values and ranges therebetween) and one or more elements of molybdenum, bismuth, niobium, tungsten, tantalum, titanium, vanadium, tungsten, manganese, zirconium, technetium, ruthenium, rhodium, hafnium, osmium, copper, iridium, and yttrium. In another non-limiting embodiment, the metal powder used to form the near net or final part includes 25-95 wt. % rhenium (and all values and ranges therebetween), and one or more of calcium, carbon, chromium, cobalt, copper, gold, hafnium, iridium, iron, lanthanum, lanthanum oxide, lead, magnesium, manganese, molybdenum, nickel, niobium, osmium, platinum, rare earth metals, rhodium, ruthenium, silver, tantalum, technetium, titanium, tungsten, vanadium, yttrium, zinc, zirconium, and/or alloys of one or more of such components.

In accordance with another and/or alternative non-limiting aspect of the present disclosure, there is optionally provided a press of near net or finished part composite. The process of pressing metals into near net of finished parts is well established; however, pressing a composite structure formed of metal powder and polymer for purposes of making complex part geometries and foam like structures is new. Similarly, using a pressing process to impart particular biologic substances into the metal matrix is also new. In one non-limiting embodiment, there is provided a process of creating a metal part with pre-defined voids to create a trabecular or foam structure composed of mixing a metal and polymer powder, and then pressing the powder into a finished part or semi-finished green part, and then sintering the part under which conditions the polymer leaves the metal behind through a process of thermal degradation of the polymer. The resulting part has a porosity associated with the size of the polymer particles as well as the homogeneity of the mixture upon pressing prior to sintering. In another non-limiting embodiment, there is provided a process by which a residual of the polymer is left behind after thermal degradation (on the metal substrate) and the polymer residual has some desired biological affect (e.g., masking the metal from the body by encapsulation, promotion of cellular attachment and growth). The polymer and metal powders can be of varying sizes to create a multiplied of voids—some large, creating a pathway for cellular growth, and some small, creating a ruff surface to promote cellular attachment.

In accordance with another and/or alternative non-limiting aspect of the present disclosure, when the refractory metal alloy or the metal alloy that includes at least 15 awt. % rhenium is formed into a blank, the shape and size of the blank is non-limiting. When the refractory metal alloy or the metal alloy that includes at least 15 awt. % rhenium is formed into a rod or tube, the rod or tube generally has a length of about 48 inches or less (e.g., 0.1-48 inches and all values and ranges therebetween); however, longer lengths can be formed. In one non-limiting arrangement, the length of the rod or tube is about 8-20 inches. The average outer diameter of the rod or tube is generally less than about 2 inches (i.e., less than about 3.14 sq. in. cross-sectional area), more typically less than about 1 inch outer diameter, and even more typically no more than about 0.5 inch outer diameter; however, larger rod or tube diameter sizes can be formed. In one non-limiting configuration for a tube, the tube has an inner diameter of about 0.31 inch plus or minus about 0.002 inch and an outer diameter of about 0.5 inch plus or minus about 0.002 inch. The wall thickness of the tube is about 0.095 inch plus or minus about 0.002 inch. As can be appreciated, this is just one example of many different sized tubes that can be formed. In one non-limiting process, the near net frame of the medical device or portion of the medical device (e.g., frame of the medical device, etc.), blank, rod, tube, etc. In one non-limiting process, the near net medical device or portion of the medical device (e.g., frame of the medical device, etc.), blank, rod, tube, etc., can be formed from one or more ingots of metal or refractory metal alloy or the metal alloy that includes at least 15 awt. % rhenium. In one non-limiting process, an arc melting process (e.g., vacuum arc melting process, etc.) can be used to form the near net medical device or portion of the medical device (e.g., frame of the medical device, etc.), blank, rod, tube, etc. In another non-limiting process, rhenium powder and tungsten powder and optionally molybdenum powder can be placed in a crucible (e.g., silica crucible, etc.) and heated under a controlled atmosphere (e.g., vacuum environment, carbon monoxide environment, hydrogen and argon environment, helium, argon, etc.) by an induction melting furnace to form the near net medical device or portion of the medical device (e.g., frame of the medical device, etc.), blank, rod, tube, etc. As can be appreciated, other metal particles can be used to form other refractory metal alloys or metal alloys that includes at least 15 awt. % rhenium (e.g., molybdenum alloys, rhenium alloys, MoRe alloys, MoReCr alloys, FeCrMoCB alloys, WCu alloys, WRe alloys, ReCr alloys, MoReTa alloy, MoReTi alloy, ReCr alloy, W alloy, Ta alloy, Nb alloy, etc.) by various processes such as melting, sintering, particle compression plus heat, etc. It can be appreciated that other or additional processes can be used to form the refractory metal alloy or the metal alloy that includes at least 15 awt. % rhenium. When a tube of refractory metal alloy or the metal alloy that includes at least 15 awt. % rhenium is to be formed, a close-fitting rod can be used during the extrusion process to form the tube; however, this is not required. In another and/or additional non-limiting process, the tube of the refractory metal alloy or the metal alloy that includes at least 15 awt. % rhenium can be formed from a strip or sheet of refractory metal alloy or the metal alloy that includes at least 15 awt. % rhenium. The strip or sheet of refractory metal alloy or the metal alloy that includes at least 15 awt. % rhenium can be formed into a tube by rolling the edges of the sheet or strip and then welding together the edges of the sheet or strip. The welding of the edges of the sheet or strip can be accomplished in several ways such as, but not limited to, a) holding the edges together and then e-beam welding the edges together in a vacuum, b) positioning a thin strip of refractory metal alloy or the metal alloy that includes at least 15 awt. % rhenium above and/or below the edges of the rolled strip or sheet to be welded, then welding the one or more strips along the rolled strip or sheet edges, and then grinding off the outer strip, or c) laser welding the edges of the rolled sheet or strip in a vacuum, oxygen reducing atmosphere, or inert atmosphere. In still another and/or additional non-limiting process, the near net frame of the medical device or portion of the medical device (e.g., frame of the medical device, etc.), blank, rod, tube, etc. of the refractory metal alloy or the metal alloy that includes at least 15 awt. % rhenium is formed by consolidating metal powder. In this process, fine particles of metal (e.g., Re, W, Mo, Ti, Cu, Ni, Cr, etc.) along with any additives are mixed to form a homogenous blend of particles. Typically, the average particle size of the metal powders is less than about 200 mesh (e.g., less than 74 microns; 2-74 microns and all values and ranges therebetween). A larger average particle size can interfere with the proper mixing of the metal powders and/or adversely affect one or more physical properties of the near net frame of the medical device or portion of the medical device (e.g., frame of the medical device, etc.), blank, rod, tube, etc. formed from the metal powders. In one non-limiting embodiment, the average particle size of the metal powders is less than about 230 mesh (e.g., less than 63 microns). In another and/or alternative non-limiting embodiment, the average particle size of the metal powders is about 2-63 microns, and more particularly about 5-40 microns. As can be appreciated, smaller average particle sizes can be used. The purity of the metal powders should be selected so the metal powders contain very low levels of carbon, oxygen, and nitrogen. Typically, the carbon content of the metal powder used to form the refractory metal alloy or the metal alloy that includes at least 15 awt. % rhenium is less than about 100 ppm, the oxygen content is less than about 50 ppm, and the nitrogen content is less than about 20 ppm. Typically, metal powder used to form the refractory metal alloy or the metal alloy that includes at least 15 awt. % rhenium has a purity grade of at least 99.9 and more typically at least about 99.95. The blend of metal powder is then pressed together to form a solid solution of the refractory metal alloy or the metal alloy that includes at least 15 awt. % rhenium into a near net medical device or portion of the medical device (e.g., frame of the medical device, etc.), blank, rod, tube, etc. Typically, the pressing process is by an isostatic process (i.e., uniform pressure applied from all sides on the metal powder); however other processes can be used. When the metal powders are pressed together isostatically, cold isostatic pressing (CIP) is typically used to consolidate the metal powders; however, this is not required. The pressing process can be performed in an inert atmosphere, an oxygen-reducing atmosphere (e.g., hydrogen, argon and hydrogen mixture, etc.) and/or under a vacuum; however, this is not required. The average density of the near net medical device or portion of the medical device (e.g., frame of the medical device, etc.), blank, rod, tube, etc., achieved by pressing together the metal powders is about 80-95% (and all values and ranges therebetween) of the final average density of the near net medical device or portion of the medical device (e.g., frame of the medical device, etc.), blank, rod, tube, etc., or about 70-99% (and all values and ranges therebetween) the minimum theoretical density of the refractory metal alloy or the metal alloy that includes at least 15 awt. % rhenium. Pressing pressures of at least about 300 MPa (e.g., 300-800 MPa and all values and ranges therebetween) are generally used. Generally, the pressing pressure is about 400-700 MPa; however, other pressures can be used. After the metal powders are pressed together, the pressed metal powders are sintered at a temperature of at least 1600° C. (e.g., 1600-3500° C. and all values and ranges therebetween) to partially or fully fuse the metal powders together to form the near net medical device or portion of the medical device (e.g., frame of the medical device, etc.), blank, rod, tube, etc. The sintering of the consolidated metal powder can be performed in an oxygen-reducing atmosphere (e.g., helium, argon, hydrogen, argon, and hydrogen mixture, etc.) and/or under a vacuum; however, this is not required. At the high sintering temperatures, a high hydrogen atmosphere will reduce both the amount of carbon and oxygen in the formed near net medical device or portion of the medical device (e.g., frame of the medical device, etc.), blank, rod, tube, etc. The sintered metal powder generally has an as-sintered average density of about 90-99.9% (and all values and ranges therebetween) the minimum theoretical density of the refractory metal alloy or the metal alloy that includes at least 15 awt. % rhenium. Typically, the sintered refractory metal alloy or the metal alloy that includes at least 15 awt. % rhenium has a final average density of at least about 5 gm/cc (e.g., 5-20 gm/cc and all values and ranges therebetween), and typically at least about 8.3 gm/cc, and can be up to or greater than about 16 gm/cc; however, this is not required. The density of the formed near net medical device or portion of the medical device (e.g., frame of the medical device, etc.), blank, rod, tube, etc., will generally depend on the type of refractory metal alloy or the metal alloy that includes at least 15 awt. % rhenium used.

In accordance with another and/or alternative non-limiting aspect of the present disclosure, when a solid rod of the refractory metal alloy or the metal alloy that includes at least 15 awt. % rhenium is formed, the rod is then formed into a tube prior to reducing the outer cross-sectional area or diameter of the rod. The rod can be formed into a tube by a variety of processes such as, but not limited to, cutting or drilling (e.g., gun drilling, etc.) or by cutting (e.g., EDM, EDM sinker, wire EDM, etc.) or by 3D printing. The cavity or passageway formed in the rod typically is formed fully through the rod; however, this is not required.

In still yet a further and/or alternative non-limiting aspect of the present disclosure, the near net medical device or portion of the medical device (e.g., frame of the medical device, etc.), blank, rod, tube, etc., can be resized to the desired dimension of the medical device. In one non-limiting embodiment, the cross-sectional area or diameter of the near net medical device or portion of the medical device (e.g., frame of the medical device, etc.), blank, rod, tube, etc., is reduced to a final near net medical device or portion of the medical device (e.g., frame of the medical device, etc.), blank, rod, tube, etc., dimension in a single step or by a series of steps. The reduction of the outer cross-sectional area or diameter of the near net medical device or portion of the medical device (e.g., frame of the medical device, etc.), blank, rod, tube, etc. may be obtained by centerless grinding, turning, electropolishing, drawing process, grinding, laser cutting, shaving, polishing, EDM cutting, etc. The outer cross-sectional area or diameter size of the near net medical device or portion of the medical device (e.g., frame of the medical device, etc.), blank, rod, tube, etc., can be reduced by the use of one or more drawing processes; however, this is not required. During the drawing process, care should be taken to not form micro-cracks in the near net medical device or portion of the medical device (e.g., frame of the medical device, etc.), blank, rod, tube, etc., during the reduction of the near net medical device or portion of the medical device (e.g., frame of the medical device, etc.), blank, rod, tube, etc., outer cross-sectional area or diameter.

In another and/or alternative non-limiting aspect of the present disclosure, the near net medical device or portion of the medical device (e.g., frame of the medical device, etc.), blank, rod, tube, etc. general if not reduced in cross-sectional area by more about 25% (e.g., 0.1-25% and all values and ranges therebetween) each time the near net medical device or portion of the medical device (e.g., frame of the medical device, etc.), blank, rod, tube, etc. is drawn down in size. When the near net medical device or portion of the medical device (e.g., frame of the medical device, etc.), blank, rod, tube, etc. optionally includes a nitride layer, the nitrided layer can optionally function as a lubricating surface during the drawing process to facilitate in the drawing of the near net medical device or portion of the medical device (e.g., frame of the medical device, etc.), blank, rod, tube, etc. Generally, the near net medical device or portion of the medical device (e.g., frame of the medical device, etc.), blank, rod, tube, etc. is reduced in cross-sectional area by about 0.1-20% each time the near net medical device or portion of the medical device (e.g., frame of the medical device, etc.), blank, rod, tube, etc. is drawn through a reducing mechanism. In another and/or alternative non-limiting process step, the near net medical device or portion of the medical device (e.g., frame of the medical device, etc.), blank, rod, tube, etc. is reduced in cross-sectional area by about 1-15% each time the near net medical device or portion of the medical device (e.g., frame of the medical device, etc.), blank, rod, tube, etc. is drawn through a reducing mechanism. In still another and/or alternative non-limiting process step, the near net medical device or portion of the medical device (e.g., frame of the medical device, etc.), blank, rod, tube, etc. is reduced in cross-sectional area by about 2-15% each time the near net medical device or portion of the medical device (e.g., frame of the medical device, etc.), blank, rod, tube, etc. is drawn through reducing mechanism. In yet another one non-limiting process step, the near net medical device or portion of the medical device (e.g., frame of the medical device, etc.), blank, rod, tube, etc. is reduced in cross-sectional area by about 5-10% each time the near net medical device or portion of the medical device (e.g., frame of the medical device, etc.), blank, rod, tube, etc. is drawn through reducing mechanism. In another and/or alternative non-limiting embodiment of the disclosure, the near net medical device or portion of the medical device (e.g., frame of the medical device, etc.), blank, rod, tube, etc. of refractory metal alloy or the metal alloy that includes at least 15 awt. % rhenium is drawn through a die to reduce the cross-sectional area of the near net medical device or portion of the medical device (e.g., frame of the medical device, etc.), blank, rod, tube, etc. Generally, before drawing the near net medical device or portion of the medical device (e.g., frame of the medical device, etc.), blank, rod, tube, etc. through a die, one end of the near net medical device or portion of the medical device (e.g., frame of the medical device, etc.), blank, rod, tube, etc. is narrowed down (nosed) so as to allow it to be fed through the die; however, this is not required. The tube drawing process is typically a cold drawing process or a plug drawing process through a die. When a cold drawing or mandrel drawing process is used, a lubricant (e.g., molybdenum paste, grease, etc.) is typically coated on the outer surface of the near net medical device or portion of the medical device (e.g., frame of the medical device, etc.), blank, rod, tube, etc. and the near net medical device or portion of the medical device (e.g., frame of the medical device, etc.), blank, rod, tube, etc. is then drawn though the die. Typically, little or no heat is used during the cold drawing process. After the near net medical device or portion of the medical device (e.g., frame of the medical device, etc.), blank, rod, tube, etc. has been drawn through the die, the outer surface of the near net medical device or portion of the medical device (e.g., frame of the medical device, etc.), blank, rod, tube, etc. is typically cleaned with a solvent to remove the lubricant so as to limit the amount of impurities that are incorporated in the refractory metal alloy or the metal alloy that includes at least 15 awt. % rhenium; however, this is not required. This cold drawing process can be repeated several times until the desired outer cross-sectional area or diameter, inner cross-sectional area or diameter and/or wall thickness of the near net medical device or portion of the medical device (e.g., frame of the medical device, etc.), blank, rod, tube, etc. is achieved. A plug drawing process can also or alternatively be used to size the near net medical device or portion of the medical device (e.g., frame of the medical device, etc.), blank, rod, tube, etc. The plug drawing process typically does not use a lubricant during the drawing process. The plug drawing process typically includes a heating step to heat the near net medical device or portion of the medical device (e.g., frame of the medical device, etc.), blank, rod, tube, etc. prior and/or during the drawing of the near net medical device or portion of the medical device (e.g., frame of the medical device, etc.), blank, rod, tube, etc. through the die. The elimination of the use of a lubricant can reduce the incidence of impurities being introduced into the refractory metal alloy or the metal alloy that includes at least 15 awt. % rhenium during the drawing process. During the plug drawing process, the near net medical device or portion of the medical device (e.g., frame of the medical device, etc.), blank, rod, tube, etc. can be protected from oxygen by use of a vacuum environment, a non-oxygen environment (e.g., hydrogen, argon and hydrogen mixture, nitrogen, nitrogen and hydrogen, etc.) or an inert environment. One non-limiting protective environment includes argon, hydrogen or argon and hydrogen; however, other or additional inert gasses can be used. As indicated above, the near net medical device or portion of the medical device (e.g., frame of the medical device, etc.), blank, rod, tube, etc. is typically cleaned after each drawing process to remove impurities and/or other undesired materials from the surface of the near net medical device or portion of the medical device (e.g., frame of the medical device, etc.), blank, rod, tube, etc.; however, this is not required. Typically, the near net medical device or portion of the medical device (e.g., frame of the medical device, etc.), blank, rod, tube, etc. should be shielded from oxygen and nitrogen when the temperature of the near net medical device or portion of the medical device (e.g., frame of the medical device, etc.), blank, rod, tube, etc. is increased to above 500° C., and typically above 450° C., and more typically above 400° C.; however, this is not required. When the near net medical device or portion of the medical device (e.g., frame of the medical device, etc.), blank, rod, tube, etc. is heated to temperatures above about 400-500° C., the near net medical device or portion of the medical device (e.g., frame of the medical device, etc.), blank, rod, tube, etc. tends to begin forming nitrides and/or in the presence of nitrogen and oxygen. In these higher temperature environments, a hydrogen environment, an argon and hydrogen environment, etc. is generally used. When the near net medical device or portion of the medical device (e.g., frame of the medical device, etc.), blank, rod, tube, etc. is drawn at temperatures below 400-500° C., the near net medical device or portion of the medical device (e.g., frame of the medical device, etc.), blank, rod, tube, etc. can be exposed to air with little or no adverse effects; however, an inert or slightly reducing environment is generally more desirable.

In another and/or alternative non-limiting aspect of the present disclosure, the near net medical device or portion of the medical device (e.g., frame of the medical device, etc.), blank, rod, tube, etc. is cooled after being annealed; however, this is not required. Generally, the near net medical device or portion of the medical device (e.g., frame of the medical device, etc.), blank, rod, tube, etc. is cooled at a fairly quick rate after being annealed so as to inhibit or prevent the formation of a sigma phase in the refractory metal alloy or the metal alloy that includes at least 15 awt. % rhenium; however, this is not required. Generally, the near net medical device or portion of the medical device (e.g., frame of the medical device, etc.), blank, rod, tube, etc. is cooled at a rate of at least about 50° C. per minute (e.g., 50-500° C. per minute and all values and ranges therebetween) after being annealed, typically at least about 75° C. per minute after being annealed, more typically at least about 100° C. per minute after being annealed, even more typically about 100-400° C. per minute after being annealed, still even more typically about 150-350° C. per minute after being annealed, and yet still more typically about 200-300° C. per minute after being annealed, and still yet even more typically about 250-280° C. per minute after being annealed; however, this is not required.

In another and/or alternative non-limiting aspect of the present disclosure, the near net medical device or portion of the medical device (e.g., frame of the medical device, etc.), blank, rod, tube, etc. is annealed after one or more drawing processes. The refractory metal alloy or the metal alloy that includes at least 15 awt. % rhenium blank, rod, tube, etc. can be annealed after each drawing process or after a plurality of drawing processes. The refractory metal alloy or the metal alloy that includes at least 15 awt. % rhenium blank, rod, tube, etc. is typically annealed prior to about a 60% cross-sectional area size reduction of the refractory metal alloy or the metal alloy that includes at least 15 awt. % rhenium blank, rod, tube, etc. In other words, the near net medical device or portion of the medical device (e.g., frame of the medical device, etc.), blank, rod, tube, etc. should not be reduced in cross-sectional area by more than 60% before being annealed (e.g., 0.1-60% reduction and all values and ranges therebetween). A too-large reduction in the cross-sectional area of the refractory metal alloy or the metal alloy that includes at least 15 awt. % rhenium blank, rod, tube, etc. during the drawing process prior to the near net medical device or portion of the medical device (e.g., frame of the medical device, etc.), blank, rod, tube, etc. being annealed can result in micro-cracking of the near net medical device or portion of the medical device (e.g., frame of the medical device, etc.), blank, rod, tube, etc. In one non-limiting processing step, the refractory metal alloy or the metal alloy that includes at least 15 awt. % rhenium blank, rod, tube, etc. is annealed prior to about a 50% cross-sectional area size reduction of the refractory metal alloy or the metal alloy that includes at least 15 awt. % rhenium blank, rod, tube, etc. In another and/or alternative non-limiting processing step, the refractory metal alloy or the metal alloy that includes at least 15 awt. % rhenium blank, rod, tube, etc. is annealed prior to about a 45% cross-sectional area size reduction of the refractory metal alloy or the metal alloy that includes at least 15 awt. % rhenium blank, rod, tube, etc. In accordance with another and/or alternative non-limiting aspect of the present disclosure, the refractory metal alloy or the metal alloy that includes at least 15 awt. % rhenium blank, rod, tube, etc. is annealed prior to about a 1-45% cross-sectional area size reduction of the refractory metal alloy or the metal alloy that includes at least 15 awt. % rhenium blank, rod, tube, etc. In yet another and/or alternative non-limiting processing step, the refractory metal alloy or the metal alloy that includes at least 15 awt. % rhenium blank, rod, tube, etc. is annealed prior to about a 5-30% cross-sectional area size reduction of the refractory metal alloy or the metal alloy that includes at least 15 awt. % rhenium blank, rod, tube, etc. In still yet another and/or alternative non-limiting processing step, the refractory metal alloy or the metal alloy that includes at least 15 awt. % rhenium blank, rod, tube, etc. is annealed prior to about a 5-15% cross-sectional area size reduction of the refractory metal alloy or the metal alloy that includes at least 15 awt. % rhenium blank, rod, tube, etc.

In accordance with another and/or alternative non-limiting aspect of the present disclosure, when the near net medical device or portion of the medical device (e.g., frame of the medical device, etc.), blank, rod, tube, etc. is annealed, the near net medical device or portion of the medical device (e.g., frame of the medical device, etc.), blank, rod, tube, etc. is typically heated to a temperature of about 500-1700° C. (and all values and ranges therebetween) for a period of about 1-200 minutes (and all values and ranges therebetween); however, other temperatures and/or times can be used. In one non-limiting processing step, the near net medical device or portion of the medical device (e.g., frame of the medical device, etc.), blank, rod, tube, etc. is annealed at a temperature of about 1000-1600° C. for about 2-100 minutes. In another non-limiting processing step, the near net medical device or portion of the medical device (e.g., frame of the medical device, etc.), blank, rod, tube, etc. is annealed at a temperature of about 1100-1500° C. for about 5-30 minutes. The annealing process typically occurs in an inert environment or an oxygen-reducing environment so as to limit the amount of impurities that may embed themselves in the refractory metal alloy or the metal alloy that includes at least 15 awt. % rhenium during the annealing process. One non-limiting oxygen-reducing environment that can be used during the annealing process is a hydrogen environment; however, it can be appreciated that a vacuum environment can be used or one or more other or additional gasses can be used to create the oxygen-reducing environment. At the annealing temperatures, a hydrogen-containing atmosphere can further reduce the amount of oxygen in the near net medical device or portion of the medical device (e.g., frame of the medical device, etc.), blank, rod, tube, etc. The chamber in which the near net medical device or portion of the medical device (e.g., frame of the medical device, etc.), blank, rod, tube, etc. is annealed should be substantially free (e.g., 0-50 ppm and all value and ranges therebetween) of impurities (e.g., carbon, oxygen, nitrogen, etc.) so as to limit the amount of impurities that can embed themselves in the near net medical device or portion of the medical device (e.g., frame of the medical device, etc.), blank, rod, tube, etc. during the annealing process. The annealing chamber typically is formed of a material that will not impart impurities to the near net medical device or portion of the medical device (e.g., frame of the medical device, etc.), blank, rod, tube, etc. as the near net medical device or portion of the medical device (e.g., frame of the medical device, etc.), blank, rod, tube, etc. is being annealed. A non-limiting material that can be used to form the annealing chamber includes, but is not limited to, molybdenum, rhenium, tungsten, molybdenum TZM alloy, cobalt, chromium, ceramic, etc. When the near net medical device or portion of the medical device (e.g., frame of the medical device, etc.), blank, rod, tube, etc. is restrained in the annealing chamber, the restraining apparatuses that are used to contact the near net medical device or portion of the medical device (e.g., frame of the medical device, etc.), blank, rod, tube, etc. are typically formed of materials that will not introduce impurities to the refractory metal alloy or the metal alloy that includes at least 15 awt. % rhenium during the processing of the near net medical device or portion of the medical device (e.g., frame of the medical device, etc.), blank, rod, tube, etc. Non-limiting examples of materials that can be used to at least partially form the restraining apparatuses include, but are not limited to, molybdenum, titanium, yttrium, zirconium, rhenium, cobalt, chromium, tantalum, and/or tungsten. In one non-limiting embodiment, when the refractory metal alloy or the metal alloy that includes at least 15 awt. % rhenium is exposed to temperatures above 150° C. for any process step including annealing, the materials that contact the refractory metal alloy or the metal alloy that includes at least 15 awt. % rhenium during the processing of the refractory metal alloy or the metal alloy that includes at least 15 awt. % rhenium are typically made from chromium, cobalt, molybdenum, rhenium, tantalum and/or tungsten. When the refractory metal alloy or the metal alloy that includes at least 15 awt. % rhenium is processed at lower temperatures (i.e., 150° C. or less), materials made from Teflon™ parts can also or alternatively be used.

In accordance with another and/or alternative non-limiting aspect of the present disclosure, the parameters for annealing can be changed as the near net medical device or portion of the medical device (e.g., frame of the medical device, etc.), blank, rod, tube, etc. as the cross-sectional area or diameter; and/or wall thickness of the near net medical device or portion of the medical device (e.g., frame of the medical device, etc.), blank, rod, tube, etc. are changed. It has been found that good grain size characteristics of the near net medical device or portion of the medical device (e.g., frame of the medical device, etc.), blank, rod, tube, etc. can be achieved when the annealing parameters are varied as the parameters of the near net medical device or portion of the medical device (e.g., frame of the medical device, etc.), blank, rod, tube, etc. change. For example, as the wall thickness is reduced, the annealing temperature is correspondingly reduced; however, the times for annealing can be increased. As can be appreciated, the annealing temperatures of the near net medical device or portion of the medical device (e.g., frame of the medical device, etc.), blank, rod, tube, etc. can be decreased as the wall thickness decreases, but the annealing times can remain the same or also be reduced as the wall thickness reduces. After each annealing process, the grain size of the metal in the near net medical device or portion of the medical device (e.g., frame of the medical device, etc.), blank, rod, tube, etc. should be no greater than 4 ASTM. Generally, the grain size range is about 4-20 ASTM (and all values and ranges therebetween). It is believed that as the annealing temperature is reduced as the wall thickness reduces, small grain sizes can be obtained. The grain size of the metal in the near net medical device or portion of the medical device (e.g., frame of the medical device, etc.), blank, rod, tube, etc. should be as uniform as possible. In addition, the sigma phase of the metal in the near net medical device or portion of the medical device (e.g., frame of the medical device, etc.), blank, rod, tube, etc. should be as reduced as much as possible. The sigma phase is a spherical, elliptical or tetragonal crystalline shape in the refractory metal alloy or the metal alloy that includes at least 15 awt. % rhenium. After the final drawing of the near net medical device or portion of the medical device (e.g., frame of the medical device, etc.), blank, rod, tube, etc., a final annealing of the near net medical device or portion of the medical device (e.g., frame of the medical device, etc.), blank, rod, tube, etc. can be done for final strengthening of the near net medical device or portion of the medical device (e.g., frame of the medical device, etc.), blank, rod, tube, etc.; however, this is not required. This final annealing process, when used, generally occurs at a temperature of about 500-1600° C. (and all values and ranges therebetween) for at least about 1 minute; however, other temperatures and/or time periods can be used.

In accordance with another and/or alternative non-limiting aspect of the present disclosure, the use of the refractory metal alloy or the metal alloy that includes at least 15 awt. % rhenium to partially or fully form the medical device or portion of the medical device (e.g., frame of the medical device, etc.) can be used to increase the strength and/or hardness and/or durability of the medical device or portion of the medical device (e.g., frame of the medical device, etc.) as compared with stainless steel or chromium-cobalt alloys or titanium alloys; thus, less quantity of refractory metal alloy or the metal alloy that includes at least 15 awt. % rhenium can be used in the medical device or portion of the medical device (e.g., frame of the medical device, etc.) to achieve similar strengths as compared to frames of medical devices formed of different metals. As such, the resulting medical device can be made smaller and less bulky by use of the refractory metal alloy or the metal alloy that includes at least 15 awt. % rhenium without sacrificing the strength and durability of the medical device. Such a medical device can have a smaller profile, thus can be inserted in smaller areas, openings and/or passageways. The refractory metal alloy or the metal alloy that includes at least 15 awt. % rhenium also can increase the radial strength of the medical device or portion of the medical device (e.g., frame of the medical device, etc.). For instance, the thickness of the walls of the medical device or portion of the medical device (e.g., frame of the medical device, etc.) and/or the wires used to at least partially form the medical device or portion of the medical device (e.g., frame of the medical device, etc.) can be made thinner and achieve a similar or improved radial strength as compared with thicker walled frames of medical devices formed of stainless steel, titanium alloys or cobalt and chromium alloys. The refractory metal alloy or the metal alloy that includes at least 15 awt. % rhenium also can improve stress-strain properties, bendability and flexibility of the medical device or portion of the medical device (e.g., frame of the medical device, etc.), thus increase the life of the medical device. For instance, the medical device can be used in regions that subject the medical device to bending. Due to the improved physical properties of the medical device from the refractory metal alloy or the metal alloy that includes at least 15 awt. % rhenium, the medical device has improved resistance to fracturing in such frequent bending environments. In addition or alternatively, the improved bendability and/or flexibility of the medical device or portion of the medical device (e.g., frame of the medical device, etc.) due to the use of the refractory metal alloy or the metal alloy that includes at least 15 awt. % rhenium can enable the medical device to be more easily inserted into various regions of a body. For medical devices that are configured to be crimped (e.g., stents, frames of heart valves, etc.), the refractory metal alloy or the metal alloy that includes at least 15 awt. % rhenium can also reduce the degree of recoil during the crimping and/or expansion of the medical device or portion of the medical device (e.g., frame of the medical device, etc.). For example, the medical device better maintains its crimped form and/or better maintains its expanded form after expansion due to the use of the refractory metal alloy or the metal alloy that includes at least 15 awt. % rhenium. As such, when the medical device is to be mounted onto a delivery device when the medical device is crimped, the medical device better maintains its smaller profile during the insertion of the medical device into various regions of a body. Also, the medical device better maintains its expanded profile after expansion so as to facilitate in the success of the medical device in the treatment area. In addition to the improved physical properties of the medical device by use of the refractory metal alloy or the metal alloy that includes at least 15 awt. % rhenium, the refractory metal alloy or the metal alloy that includes at least 15 awt. % rhenium has improved radiopaque properties as compared to standard materials such as stainless steel or cobalt-chromium alloy, thus reducing or eliminating the need for using marker materials on the medical device. For instance, the refractory metal alloy or the metal alloy that includes at least 15 awt. % rhenium is believed to at least about 10-20% more radiopaque than stainless steel or cobalt-chromium alloy.

In accordance with another and/or alternative non-limiting aspect of the present disclosure, the refractory metal alloy or the metal alloy that includes at least 15 awt. % rhenium is subjected to a final heat treatment process along a portion or all of the longitudinal length of the piece of refractory metal alloy or the metal alloy that includes at least 15 awt. % rhenium that is to be used to partially or fully form the medical device. The final heat treatment process is used to soften the refractory metal alloy or the metal alloy that includes at least 15 awt. % rhenium (e.g., increase flexibility, increase bendability, reduce the yield strength and/or ultimate tensile strength). As the distance increase into the heated section of the piece of refractory metal alloy or the metal alloy that includes at least 15 awt. % rhenium, the force decreases to break the piece of refractory metal alloy or the metal alloy that includes at least 15 awt. % rhenium. This is a result of the yield strength and/or ultimate tensile strength decreasing in the piece of refractory metal alloy or the metal alloy that includes at least 15 awt. % rhenium as the temperature increases during the final heat treatment of the piece of the refractory metal alloy or the metal alloy that includes at least 15 awt. % rhenium. During the final heat treatment, there is realignment of the crystalline structure of the piece of refractory metal alloy or the metal alloy that includes at least 15 awt. % rhenium rod and a stress relief that may have been a result of prior cold working and/or tempering of the piece of refractory metal alloy or the metal alloy that includes at least 15 awt. % rhenium. Generally, there is no quenching of the refractory metal alloy or the metal alloy that includes at least 15 awt. % rhenium after the final heat treatment process. As such, the heated refractory metal alloy or the metal alloy that includes at least 15 awt. % rhenium can be cooled by mere exposure to the ambient temperature of the atmosphere about the heated refractory metal alloy or the metal alloy that includes at least 15 awt. % rhenium (e.g., non-oxidizing gas environment at a temperature of 10-100° C. and all values and ranges therebetween, inert gas environment at a temperature of 10-100° C. and all values and ranges therebetween, air environment at a temperature of 10-100° C. and all values and ranges therebetween, etc.). Generally, the rate of cooling after the final heat treatment step is less than 100° C./s (e.g., less than 1-100° C./s and all values and ranges therebetween), and typically less than 50° C./s, and more typically less than 25° C./s. The final heat treatment can be uniform or non-uniform along the longitudinal length of the piece of refractory metal alloy or the metal alloy that includes at least 15 awt. % rhenium that is to be used to partially or fully form the medical device. The time of the final heat treatment at different locations along the longitudinal length of the piece of refractory metal alloy or the metal alloy that includes at least 15 awt. % rhenium that is to be used to partially or fully form the medical device can be the same or different. The temperature of the final heat treatment at different locations along the longitudinal length of the piece of refractory metal alloy or the metal alloy that includes at least 15 awt. % rhenium that is to be used to partially or fully form the medical device can be the same or different. In one non-limited embodiment, the portion of the piece of refractory metal alloy or the metal alloy that includes at least 15 awt. % rhenium that is to be subjected to the final heat treatment is a) exposed to a uniform temperature for a uniform period of time, and b) is allowed to cool at substantially the same cooling rate, and c) is not subjected to quenching.

In accordance with another and/or alternative non-limiting aspect of the present disclosure, the refractory metal alloy or the metal alloy that includes at least 15 awt. % rhenium is subjected to a final heat treatment process along a portion of the longitudinal length of the piece of refractory metal alloy or the metal alloy that includes at least 15 awt. % rhenium that is to be used to partially or fully form the medical device, and a heat shielding arrangement is used to prevent a portion of the piece of metal alloy from being subjected to the final heat treatment. The type of heat shield is non-limiting. In one non-limiting arrangement, the heat shielding is accomplishing by only inserting a portion of the piece of refractory metal alloy or the metal alloy that includes at least 15 awt. % rhenium into an oven. In one non-limiting configuration, an opening is provided in an outer surface of the heating oven (e.g., sand furnace, etc.) that enables a portion of the piece of refractory metal alloy or the metal alloy that includes at least 15 awt. % rhenium to be inserted into the interior of the heating oven. The location of the opening is non-limiting. In another non-limiting arrangement, the opening is in the oven cover or furnace cover. The piece of refractory metal alloy or the metal alloy that includes at least 15 awt. % rhenium (e.g., MoRe alloy, etc.) can be in the formed of a rod; however, this is not required. Such rod can be used a rod for spinal surgery; however, the rod can be used in other medical procedure. The rod can have a generally uniform cross-sectional shape and area along most (50-99.9% and all values and ranges therebetween) or all of the longitudinal length of the rod. In one non-limiting arrangement, the rod portion that is located in the heating oven is exposed to temperatures of up to 1000° C. (e.g., 100-1000° C. and all values and ranges therebetween). In one particular non-limiting arrangement, the rod portion that was located in the furnace was initially exposed to a temperature of 10-250° C. (and all values and ranges therebetween, 90-160° C.) (e.g., Preheat step), and then the heat was increased to 500-1000° C. (and all values and ranges therebetween, 550-700° C.) over a period of 0.5-10 hours (and all values and ranges therebetween, 1-3 hours), and thereafter the heat in the heating oven was maintained at the maximum temperature for 1-15 hours (and all values and ranges therebetween, 2-10 hours). The heating of the rod in the heating furnace resulted in the heated portion having a reduced yield strength and/or reduced ultimate tensile strength, thereby making the portion of the rod that was heated more flexible or bendable than the portion of the rod that was not inserted into the heating furnace. During the final heating of the rod, a portion of the rod can be withdrawn from the heating oven during the heating period such that a lower portion of the rod is heated for a longer period of time in the heating oven than an intermediate portion of the rod. Such heating of the rod can result in the lower portion of the rod that is heated longer to have a lower yield strength and/or ultimate tensile strength, than the intermediate portion of the rod, thereby making the lower portion of the rod more flexible or bendable than the intermediate portion and the top portion that was not inserted into the heating oven. Generally, there is no quenching of the refractory metal alloy or the metal alloy that includes at least 15 awt. % rhenium after the final heat treatment process. As such, the heated refractory metal alloy or the metal alloy that includes at least 15 awt. % rhenium can be cooled by mere exposure to the ambient temperature of the atmosphere about the heated refractory metal alloy or the metal alloy that includes at least 15 awt. % rhenium (e.g., non-oxidizing gas environment at a temperature of 10-100° C. and all values and ranges therebetween, inert gas environment at a temperature of 10-100° C. and all values and ranges therebetween, air environment at a temperature of 10-100° C. and all values and ranges therebetween, etc.). Generally, the rate of cooling after the final heat treatment step is less than 100° C./s (e.g., less than 1-100° C./s and all values and ranges therebetween), and typically less than 50° C./s, and more typically less than 25° C./s.

In accordance with another and/or alternative non-limiting aspect of the present disclosure, the rod that is being subjected to the final heating can be up to 40 inches long (e.g., 2-40 inches and all values and ranges therebetween; up to 20 inches, etc.); and there after cut to desired lengths (e.g., 20-500 mm [and all values and ranges therebetween] with a diameter of 3-8 mm [and all values and ranges therebetween]). It will be appreciated that other rod lengths can be used. As can be appreciated, the cut portions of the rod can be a) rod portions that were subjected to the final heating process, b) rod portions that were not subject to the final heating process, or c) rod portions where a portion was subjected to the final heating process and a portion that was not subjected to the final heating process. The physical properties of the rod portions of a) and b) can be uniform or substantially uniform along the longitudinal length of the rod portions, and the physical properties of rod portions c) likely vary along the longitudinal length of the rod portions.

In accordance with another and/or alternative non-limiting aspect of the present disclosure, rods having different degrees of flexibility or bendability, but having the same cross-sectional size and shape along the longitudinal length of the rod can be formed for use in a certain medical procedure. As such, the medical professional, during a medical procedure (e.g., spinal surgery) can use the same sized spinal rods in the procedure, but merely select a rod having the desired flexibility or bendability for use in a certain region of the spine. As such, the need to use ground rods having different diameters in a medical procedure can be eliminated. Therefore, the support structure for use with the spinal rods can be uniformly sized and single size screws (e.g., pedicle screws) used to secure the rods to the other spinal structures can be used, there by simplifying the medical procedure, and reducing mistakes during the medical procedure. The spinal rods can be color coded (e.g., color coating, etc.) or have other or additional markings to indicate the flexibility or bendability of the rod. Such color coding or markings can be used to easily identify to the medical professional which spinal rod should be selected for use in a particular location of the spinal surgery. As such, multiple spinal rods having the same cross-sectional size and shape along the longitudinal length of the rod, but have different yield strengths and/or ultimate tensile strengths, can be represented by color coding or markings so as to easily identify to the medical professional the rods that are available to the medical professional during a medical procedure, and the color coding or markings enable the medical professional to quickly, easily and accurately identify the desired spinal rod for used in a particular location of the spinal surgery. Historically, thinner diameter rods were used in spinal surgery at the top portion of the spinal and larger diameter rods were used in spinal surgery at the lower portion of the spine. The smaller diameter rods allowed for more flexibility or bendability of the spine after spinal surgery at the location of the spine nearer the head wherein more flexibility or bendability was desired. However, thicker diameter spinal rods where desired at the lower portion of the spine since less flexibility or bendability was desired at such location after spinal surgery. Intermediate thick diameter spinal rods were desired at the location between the top and bottom portions of the spine since intermediate flexibility or bendability was desired at such location after spinal surgery. As such, multiple diameter rods had to be provided during a surgical procedure so that the proper rod with the desired flexibility or bendability was used. One issue with the grinding the rods to form different diameter rods was that the screws (e.g., pedicle screws) used to properly secure the rods needed to be changed based on the diameter of the rod. The top portion of the screw includes a rod opening of a particular diameter and shape. This proper screw selection could make is difficult for the surgeon to determine which screw was the proper screw for use with a certain diameter rod. By being able to use uniform diameter rods that can have variable flexibility or bendability in accordance with the present disclosure can save the surgeon time, reduce inventory, reduce the chance of a surgical error, and result in a better operation for the patient (more uniform alignment of the spine).

In accordance with another and/or alternative non-limiting aspect of the present disclosure, there is provided a rod or post wherein different portions of the rod or post along a longitudinal length of the rod or post is heat treated at different amounts so that the rod or post has a plurality of different physical properties along the longitudinal length of a rod or post. In one non-limiting configuration, 10-50% (and all values and ranges therebetween) of the longitudinal length of the rod or post has been heat treated a different amount than 10-50% (and all values and ranges therebetween) of the other portion of the rod or post, and wherein the rod or post is formed of a uniform composition, and the rod or post as a uniform cross-sectional shape and area along the longitudinal length of the rod or post, and the physical properties of the rod or post (e.g., flexibility or bendability, flexure stress at break, displacement at break, force at break, etc.) are different along different regions along the longitudinal length of the rod or post.

In accordance with another and/or alternative non-limiting aspect of the present disclosure, there is provided a set of rods or posts for use in an orthopedic procedure or spinal procedure, wherein a plurality of the rods or posts have different physical properties (e.g., flexure stress at break, displacement at break, force at break, etc.) along 60-100% (and all values and ranges therebetween) of the longitudinal length of the rods, and each of the rods are color coded and/or otherwise marked to indicate the relative physical properties of each of the rods so that a physician can visually identify a rod or post having a particular physical property for used in an orthopedic or spinal procedure. In such an arrangement, the physician can select the rod or post that has a desired bendability and/or flexibility for use in a certain medical procedure and in a certain location in a patient.

In accordance with another and/or alternative non-limiting aspect of the present disclosure, there is provided a method for manufacturing a plurality of medical devices having the same or similar size, shape and configuration and that are formed of the same refractory metal alloy or the metal alloy that includes at least 15 awt. % rhenium, but have different physical properties with regard to at least the flexibility or bendability, yield strength and/or ultimate tensile strength. In one non-limiting arrangement, there is provided a plurality of devices (e.g., medical devices [e.g., spinal rods, spinal screws (e.g., pedicle screws), spinal posts, etc.], medical tools, etc.) that a) are formed of the same metal alloy (e.g., refractory metal alloy [e.g., MoRe alloy, Re alloy, Mo alloy, Ta alloy, W alloy, etc.], metal alloy that includes at least 15 awt. % rhenium, etc.), b) are the exact same size, shape and configuration or have a similar size, shape and configuration (e.g., devices have the exact same size, shape and configuration along 40-100% of the longitudinal length of the device [and all values and ranges therebetween]), c) have different physical properties with regard to at least the flexibility or bendability, yield strength and/or ultimate tensile strength due at least in part to the subjecting of the metal alloy on the plurality of devices to different final heating processes, and d) that optionally have one or more markings (e.g., color coatings, etchings on outer surface, symbols on outer surface, etc.) that are used to visually indicate the properties or relative properties of the plurality of the devices. In another non-limiting arrangement, there is provided a plurality of medical devices in the form of spinal rods, spinal screws, or spinal posts, etc. that a) are formed of the same metal alloy (e.g., refractory metal alloy, metal alloy that includes at least 15 awt. % rhenium, etc.), b) have a similar size, shape and configuration along 60-100% (and all values and ranges therebetween) of the longitudinal length of the medical device, c) have different physical properties with regard to at least the flexibility or bendability, yield strength and/or ultimate tensile strength along one or more portions of the medical device along the longitudinal length of the medical device (e.g., top portion of medical device has different physical properties from the middle portion and/or bottom portion of the medical device, bottom portion of the medical device has different physical properties from the middle portion of the medical device, etc.) due at least in part to the subjecting of the metal alloy on the plurality of medical devices to different final heating processes, and d) have one or more markings (e.g., color coatings, etchings on outer surface, symbols on outer surface, etc.) that are used to visually indicate the properties or relative properties of the plurality of the medical devices.

In accordance with another and/or alternative non-limiting aspect of the present disclosure, there is provided a method for using medical devices in the form of spinal rods or spinal posts in a spinal surgery procedure comprising: a) provided a plurality of spinal rods or spinal posts that i) are formed of the same metal alloy (e.g., refractory metal alloy [e.g., MoRe alloy, Re alloy, Mo alloy, Ta alloy, W alloy, etc.], metal alloy that includes at least 15 awt. % rhenium, etc.), ii) have a body member that is generally cylindrical in shape and has a constant cross-sectional shape and cross-sectional size o diameter along 60-100% (and all values and ranges therebetween) of the longitudinal length of the body member, iii) the body member of a plurality of the spinal rods or spinal posts has different physical properties with regard to at least the flexibility or bendability, yield strength and/or ultimate tensile strength due at least in part to the subjecting of the metal alloy on the body member of the plurality of the spinal rods or spinal posts to different final heating processes, and iv) the spinal rod or spinal posts optionally have one or more markings (e.g., color coatings, etchings on outer surface, symbols on outer surface, etc.) that are used to visually indicate the properties or relative properties of one or more or all of the spinal rods or spinal posts; b) identifying the location that a plurality of spinal rods or spinal posts are to be used in the spinal surgical procedure; c) determining the desired flexibility or bendability of each spinal rod or post that is be used in a certain location of the spinal during the spinal surgical procedure; d) selecting a spinal rod or post that has the desired flexibility or bendability (and wherein such selection is optionally based on a marking on the spinal rod or spinal post; e) selecting the spinal bone screws (e.g., pedicle screws) for use in the spinal procedure, and wherein each of the spinal bone screws include a top portion that includes a post opening that is configured to receive a portions of the spinal rod or spinal post so that the spinal rod or spinal post can be secured in the post opening to thereby secure the spinal rod or spinal post to the top portion of the spinal bone screw, and wherein the shape, size and configuration of the posting poring for a plurality of all of the spinal bone screws is the same; f) inserting two or more spinal bone screws into the bone (e.g., spine, etc.) of a patient; and g) securing the selected spinal rod or spinal post that have the desired flexibility or bendability to the spinal bone screws during the spinal surgical procedure.

In accordance with another and/or alternative non-limiting aspect of the present disclosure, there is provided a method of providing spinal fusion components to a medical provider or healthcare system comprising; a) providing a plurality of spinal screws or pedicle screws, each of the spinal screws includes a body portion that is configured to be inserted into the spine and a head portion that is connected to the body portion and which head portion is configured to include a post opening and a securing arrangement, wherein the longitudinal length of the body portion is 20-80 mm (and all values and ranges therebetween), wherein the post opening is designed to allow a portion of a spinal rod or post to be inserted there through, wherein the configuration of the post opening is the same of the spinal screw irrespective of the longitudinal length of the body, and the securing arrangement is designed to secure a spinal rod or spinal post in the post opening; b) provided a plurality of spinal rods or spinal posts that i) are formed of the same metal alloy (e.g., refractory metal alloy [e.g., MoRe alloy, Re alloy, Mo alloy, Ta alloy, W alloy, etc.], metal alloy that includes at least 15 awt. % rhenium, etc.), ii) have a body member that is generally cylindrical in shape and has a constant cross-sectional shape and cross-sectional size o diameter along 60-100% (and all values and ranges therebetween) of the longitudinal length of the body member, iii) the body member of a plurality of the spinal rods or spinal posts has different physical properties with regard to at least the flexibility or bendability, yield strength and/or ultimate tensile strength due at least in part to the subjecting of the metal alloy on the body member of the plurality of the spinal rods or spinal posts to different final heating processes, and iv) the spinal rod or spinal posts optionally have one or more markings (e.g., color coatings, etchings on outer surface, symbols on outer surface, etc.) that are used to visually indicate the properties or relative properties of one or more or all of the spinal rods or spinal posts; c) enabling the medical provider or healthcare system to use a plurality of provided spinal screws for use in a particular medical procedure; d) enabling the medical provider or healthcare system to use one or more of the provided spinal rods or posts for use in the particular medical procedure, and wherein the obtained spinal rod or spinal post will properly fit in and be secured to the opening of the spinal screw.

In accordance with another and/or alternative non-limiting aspect of the present disclosure, the use of the refractory metal alloy or the metal alloy that includes at least 15 awt. % rhenium to form all or a portion of the medical device can result in several advantages over medical devices formed from other materials. These advantages include, but are not limited to:

The refractory metal alloy or the metal alloy that includes at least 15 awt. % rhenium has increased strength and/or hardness as compared with stainless steel, chromium-cobalt alloys, or titanium alloys, thus a less quantity of refractory metal alloy or the metal alloy that includes at least 15 awt. % rhenium can be used in the medical device to achieve similar strengths as compared to medical devices formed of different metals. As such, the resulting medical device can be made smaller and less bulky by use of the refractory metal alloy or the metal alloy that includes at least 15 awt. % rhenium without sacrificing the strength and durability of the medical device. The medical device can also have a smaller profile, thus can be inserted into smaller areas, openings, and/or passageways. The thinner struts of refractory metal alloy or the metal alloy that includes at least 15 awt. % rhenium to form the frame or other portions of the medical device can be used to form a frame or other portion of the medical device having a strength that would require thicker struts or other structures of the medical device when formed by stainless steel, chromium-cobalt alloys, or titanium alloys.

The increased strength of the refractory metal alloy or the metal alloy that includes at least 15 awt. % rhenium also results in the increased radial strength of the medical device. For instance, the thickness of the walls of the medical device can be made thinner and achieve a similar or improved radial strength as compared with thicker walled medical devices formed of stainless steel, cobalt and chromium alloy, or titanium alloy.

The refractory metal alloy or the metal alloy that includes at least 15 awt. % rhenium has improved stress-strain properties, bendability properties, elongation properties, and/or flexibility or bendability properties of the medical device compared with stainless steel and chromium-cobalt alloys, thus resulting in an increase life for the medical device. For example, the medical device can be used in regions that subject the medical device to repeated bending. Due to the improved physical properties of the medical device from the refractory metal alloy or the metal alloy that includes at least 15 awt. % rhenium, the medical device has improved resistance to fracturing in such frequent bending environments. These improved physical properties at least in part result from the composition of the refractory metal alloy or the metal alloy that includes at least 15 awt. % rhenium, the grain size of the refractory metal alloy or the metal alloy that includes at least 15 awt. % rhenium, the carbon, oxygen, and nitrogen content of the refractory metal alloy or the metal alloy that includes at least 15 awt. % rhenium, and/or the carbon/oxygen ratio of the refractory metal alloy or the metal alloy that includes at least 15 awt. % rhenium.

The refractory metal alloy or the metal alloy that includes at least 15 awt. % rhenium has and wherein at least a portion of the medical device is configured to be crimped and/or expanded (e.g., stent, frame of heart valve, etc.), a reduced degree of recoil during the crimping and/or expansion of the medical device compared with stainless steel, chromium-cobalt alloys, or titanium alloys. The medical device formed of the refractory metal alloy or the metal alloy that includes at least 15 awt. % rhenium better maintains its crimped form and/or better maintains its expanded form after expansion due to the use of the refractory metal alloy or the metal alloy that includes at least 15 awt. % rhenium. As such, when the medical device is to be mounted onto a delivery device when the medical device is crimped, the medical device better maintains its smaller profile during the insertion of the medical device in a body passageway. Also, the medical device better maintains its expanded profile after expansion to facilitate in the success of the medical device in the treatment area.

The use of the refractory metal alloy or the metal alloy that includes at least 15 awt. % rhenium in the medical device results in the medical device better conforming to an irregularly shaped body passageway when expanded in the body passageway compared to a medical device formed by stainless steel, chromium-cobalt alloys, or titanium alloys.

The refractory metal alloy or the metal alloy that includes at least 15 awt. % rhenium has improved radiopaque properties compared to standard materials such as stainless steel or cobalt-chromium alloy, thus reducing or eliminating the need for using marker materials on the medical device. For example, the refractory metal alloy or the metal alloy that includes at least 15 awt. % rhenium is at least about 10-20% more radiopaque than stainless steel or cobalt-chromium alloy.

The refractory metal alloy or the metal alloy that includes at least 15 awt. % rhenium has improved fatigue ductility when subjected to cold-working compared to the cold-working of stainless steel, chromium-cobalt alloys, or titanium alloys.

The refractory metal alloy or the metal alloy that includes at least 15 awt. % rhenium has improved durability compared to stainless steel, chromium-cobalt alloys, or titanium alloys.

The refractory metal alloy or the metal alloy that includes at least 15 awt. % rhenium has improved hydrophilicity compared to stainless steel, chromium-cobalt alloys, or titanium alloys.

The refractory metal alloy or the metal alloy that includes at least 15 awt. % rhenium has reduced ion release in the body passageway compared to stainless steel, chromium-cobalt alloys, or titanium alloys.

The refractory metal alloy or the metal alloy that includes at least 15 awt. % rhenium is less of an irritant to the body than stainless steel, cobalt-chromium alloy, or titanium alloys, thus can result in reduced inflammation, faster healing, increased success rates of the medical device. When the medical device is expanded in a body passageway, some minor damage to the interior of the passageway can occur. When the body begins to heal such minor damage, the body has less adverse reaction to the presence of the refractory metal alloy or the metal alloy that includes at least 15 awt. % rhenium compared to other metals such as stainless steel, cobalt-chromium alloy, or titanium alloy.

The refractory metal alloy or the metal alloy that includes at least 15 awt. % rhenium has a magnetic susceptibility that is lower that CoCr alloy, TiAlV alloys, and/or stainless steel, thus resulting in less incidence of potential defects to the medical device or complications to the patent after implantation of the medical device when the patient is subjected to an MRI or other medical device that generates a strong magnetic field.

One non-limiting object of the present disclosure is the provision of the refractory metal alloy or the metal alloy that includes at least 15 awt. % rhenium in accordance with the present disclosure that can be used to partially or fully form a medical device.

Another and/or alternative non-limiting object of the present disclosure is the provision of a medical device that is partially or fully formed of the refractory metal alloy or the metal alloy that includes at least 15 awt. % rhenium of the present disclosure and which medical device has improved procedural success rates.

Another and/or alternative non-limiting object of the present disclosure is the provision of a method and process for forming the refractory metal alloy or the metal alloy that includes at least 15 awt. % rhenium in accordance with the present disclosure that inhibits or prevents the formation of micro-cracks during the processing of the refractory metal alloy or the metal alloy that includes at least 15 awt. % rhenium.

Another and/or alternative non-limiting object of the present disclosure is the provision of a medical device that is partially or fully formed of the refractory metal alloy or the metal alloy that includes at least 15 awt. % rhenium in accordance with the present disclosure and wherein the medical device has improved physical properties.

Another and/or alternative non-limiting object of the present disclosure is the provision of a medical device that is at least partially formed of the refractory metal alloy or the metal alloy that includes at least 15 awt. % rhenium in accordance with the present disclosure that has increased strength and/or hardness.

Another and/or alternative non-limiting object of the present disclosure is the provision of a medical device that at least partially includes the refractory metal alloy or the metal alloy that includes at least 15 awt. % rhenium in accordance with the present disclosure and which refractory metal alloy or the metal alloy that includes at least 15 awt. % rhenium enables the medical device to be formed with less material without sacrificing the strength of the medical device compared to prior medical devices.

Another and/or alternative non-limiting object of the present disclosure is the provision of a method and process for forming the refractory metal alloy or the metal alloy that includes at least 15 awt. % rhenium in accordance with the present disclosure to inhibit or prevent the formation of micro-cracks during the processing of the refractory metal alloy or the metal alloy that includes at least 15 awt. % rhenium into a medical device.

Another and/or alternative non-limiting object of the present disclosure is the provision of a method and process for forming the refractory metal alloy or the metal alloy that includes at least 15 awt. % rhenium in accordance with the present disclosure that inhibits or prevents crack propagation and/or fatigue failure of the refractory metal alloy or the metal alloy that includes at least 15 awt. % rhenium.

Another and/or alternative non-limiting object of the present disclosure is the provision of a medical device that includes a refractory metal alloy or the metal alloy that includes at least 15 awt. % rhenium having a nitriding process to form a nitrided layer on the outer surface of the refractory metal alloy or the metal alloy that includes at least 15 awt. % rhenium.

Another and/or alternative non-limiting object of the present disclosure is the provision of a medical device that includes a refractory metal alloy or the metal alloy that includes at least 15 awt. % rhenium wherein the refractory metal alloy or the metal alloy that includes at least 15 awt. % rhenium has been subjected to a swaging process.

Another and/or alternative non-limiting object of the present disclosure is the provision of a medical device that includes a refractory metal alloy or the metal alloy that includes at least 15 awt. % rhenium wherein the refractory metal alloy or the metal alloy that includes at least 15 awt. % rhenium has been subjected to a cold-working process.

Another and/or alternative non-limiting object of the present disclosure is the provision of a medical device that includes a refractory metal alloy or the metal alloy that includes at least 15 awt. % rhenium that has increased strength and/or hardness as compared with stainless steel, chromium-cobalt alloys, or titanium alloys.

Another and/or alternative non-limiting object of the present disclosure is the provision of a medical device that includes a refractory metal alloy or the metal alloy that includes at least 15 awt. % rhenium thereby requiring a less quantity of refractory metal alloy or the metal alloy that includes at least 15 awt. % rhenium to achieve similar strengths compared to medical devices formed of different metals.

Another and/or alternative non-limiting object of the present disclosure is the provision of a medical device that includes a refractory metal alloy or the metal alloy that includes at least 15 awt. % rhenium, and wherein at least a portion of the medical device is configured to be crimped and/or expanded (e.g., stent, frame of heart valve, etc.), the medical device has a smaller crimped profile as compared to medical devices formed of different metals.

Another and/or alternative non-limiting object of the present disclosure is the provision of a medical device that includes a refractory metal alloy or the metal alloy that includes at least 15 awt. % rhenium, and wherein at least a portion of the medical device is configured to be crimped and/or expanded (e.g., stent, frame of heart valve, etc.), and wherein the medical device has thinner walls and/or struts than in frames of a same shape that are formed of stainless steel, cobalt and chromium alloy or titanium alloy, and such frame formed of refractory metal alloy or the metal alloy that includes at least 15 awt. % rhenium has the same or increase radial strength when the frame is expanded form a crimped configuration to an expanded configuration as compared to such frames formed of stainless steel or cobalt and chromium alloy, or titanium alloy.

Another and/or alternative non-limiting object of the present disclosure is the provision of a medical device that includes a refractory metal alloy or the metal alloy that includes at least 15 awt. % rhenium wherein the medical device has improved stress-strain properties, bendability properties, elongation properties, and/or flexibility or bendability properties as compared to medical devices formed of stainless steel, titanium alloy, or chromium-cobalt alloys.

Another and/or alternative non-limiting object of the present disclosure is the provision of a medical device that includes a refractory metal alloy or the metal alloy that includes at least 15 awt. % rhenium wherein the medical device has an increase life as compared to medical devices formed of stainless steel, titanium alloy, or chromium-cobalt alloys.

Another and/or alternative non-limiting object of the present disclosure is the provision of a medical device that includes a refractory metal alloy or the metal alloy that includes at least 15 awt. % rhenium, and wherein at least a portion of the medical device is configured to be crimped and/or expanded (e.g., stent, frame of heart valve, etc.), and wherein the medical device has a reduced degree of recoil during the crimping and/or expansion of the medical device compared with frames of a similar size, shape and configuration that are formed of stainless steel, chromium-cobalt alloys, or titanium alloys.

Another and/or alternative non-limiting object of the present disclosure is the provision of a medical device that includes a refractory metal alloy or the metal alloy that includes at least 15 awt. % rhenium wherein the medical device better conforms to an irregularly shaped body passageway when expanded in the body passageway as compared with frames of a similar size, shape and configuration that are formed of stainless steel, chromium-cobalt alloys, or titanium alloys.

Another and/or alternative non-limiting object of the present disclosure is the provision of a medical device that includes a refractory metal alloy or the metal alloy that includes at least 15 awt. % rhenium wherein the medical device has improved fatigue ductility when subjected to cold-working as compared to the cold-working of frames of a similar size, shape and configuration that are formed of stainless steel, chromium-cobalt alloys, or titanium alloys.

Another and/or alternative non-limiting object of the present disclosure is the provision of a medical device that includes a refractory metal alloy or the metal alloy that includes at least 15 awt. % rhenium wherein the medical device has improved durability as compared to stainless steel, chromium-cobalt alloys, or titanium alloys.

Another and/or alternative non-limiting object of the present disclosure is the provision of a medical device that includes a refractory metal alloy or the metal alloy that includes at least 15 awt. % rhenium wherein the medical device has improved hydrophilicity as compared to stainless steel, chromium-cobalt alloys, or titanium alloys.

Another and/or alternative non-limiting object of the present disclosure is the provision of a medical device that includes a refractory metal alloy or the metal alloy that includes at least 15 awt. % rhenium wherein the medical device has reduced ion release in the body passageway as compared to stainless steel, chromium-cobalt alloys, or titanium alloys.

Another and/or alternative non-limiting object of the present disclosure is the provision of a medical device that includes a refractory metal alloy or the metal alloy that includes at least 15 awt. % rhenium wherein the medical device is less of an irritant to the body than stainless steel, cobalt-chromium alloy, or titanium alloys, thus can result in reduced inflammation, faster healing, and increased success rates of the medical device.

Another and/or alternative non-limiting object of the present disclosure is the provision of a medical device that includes a refractory metal alloy or the metal alloy that includes at least 15 awt. % rhenium wherein the medical device is subjected to a final heat treatment process along a portion or all of the longitudinal length of the metal alloy.

Another and/or alternative non-limiting object of the present disclosure is the provision of a medical device that includes a refractory metal alloy or the metal alloy that includes at least 15 awt. % rhenium wherein the medical device is subjected to a final heat treatment process along a portion or all of the longitudinal length of the metal alloy to soften the metal alloy (e.g., increase flexibility, increase bendability, reduce the yield strength and/or ultimate tensile strength).

Another and/or alternative non-limiting object of the present disclosure is the provision of a medical device that includes a refractory metal alloy or the metal alloy that includes at least 15 awt. % rhenium wherein the medical device is subjected to a final heat treatment process along a portion or all of the longitudinal length of the metal alloy to soften the metal alloy, and the metal alloy is not subjected to quenching after the final heat treatment process.

Another and/or alternative non-limiting object of the present disclosure is the provision of a medical device that includes a refractory metal alloy or the metal alloy that includes at least 15 awt. % rhenium wherein the medical device is subjected to a final heat treatment process is thereafter cooled by mere exposure to the ambient temperature (e.g., 55-95° F. and all values and ranges therebetween, 70-80° F., etc.) of the atmosphere about the heated metal alloy, and the cooling can optionally occur in a non-oxidizing gas environment, inert gas environment, partial or full vacuum [e.g., 0-0.5 atm.], etc.).

Another and/or alternative non-limiting object of the present disclosure is the provision of a medical device that includes a refractory metal alloy or the metal alloy that includes at least 15 awt. % rhenium wherein the medical device is subjected to a final heat treatment process along a portion of the longitudinal length of the metal alloy, and a heat shielding arrangement is used to prevent another portion of the piece of the metal alloy from being subjected to the final heat treatment.

Another and/or alternative non-limiting object of the present disclosure is the provision of a rods or posts having different degrees of flexibility or bendability, but have the same cross-sectional size and shape along the longitudinal length of the rod or post.

Another and/or alternative non-limiting object of the present disclosure is the provision of a rods or posts having different degrees of flexibility or bendability, but have the same cross-sectional size and shape along the longitudinal length of the rod or post, and the rods or posts can be color coded (e.g., color coating, etc.) or have other or additional markings to indicate the flexibility or bendability of the rod or post.

Another and/or alternative non-limiting object of the present disclosure is the provision of a plurality of devices (e.g., medical devices [e.g., spinal rods, spinal screws (e.g., pedicle screws), spinal posts, etc.], medical tools, etc.) that a) are formed of the same metal alloy (e.g., refractory metal alloy [e.g., MoRe alloy, Re alloy, Mo alloy, Ta alloy, W alloy, etc.], metal alloy that includes at least 15 awt. % rhenium, etc.), b) are the exact same size, shape and configuration or have a similar size, shape and configuration (e.g., devices have the exact same size, shape and configuration along 40-100% of the longitudinal length of the device [and all values and ranges therebetween]), c) have different physical properties with regard to at least the flexibility or bendability, yield strength and/or ultimate tensile strength due at least in part to the subjecting of the metal alloy on the plurality of devices to different final heating processes, and d) that optionally have one or more markings (e.g., color coatings, etchings on outer surface, symbols on outer surface, etc.) that are used to visually indicate the properties or relative properties of the plurality of the devices.

Another and/or alternative non-limiting object of the present disclosure is the provision of a method for using medical devices in the form of spinal rods or spinal posts in a spinal surgery procedure comprising: a) provided a plurality of spinal rods or spinal posts that i) are formed of the same metal alloy (e.g., refractory metal alloy [e.g., MoRe alloy, Re alloy, Mo alloy, Ta alloy, W alloy, etc.], metal alloy that includes at least 15 awt. % rhenium, etc.), ii) have a body member that is generally cylindrical in shape and has a constant cross-sectional shape and cross-sectional size o diameter along 60-100% (and all values and ranges therebetween) of the longitudinal length of the body member, iii) the body member of a plurality of the spinal rods or spinal posts has different physical properties with regard to at least the flexibility or bendability, yield strength and/or ultimate tensile strength due at least in part to the subjecting of the metal alloy on the body member of the plurality of the spinal rods or spinal posts to different final heating processes, and iv) the spinal rod or spinal posts optionally have one or more markings (e.g., color coatings, etchings on outer surface, symbols on outer surface, etc.) that are used to visually indicate the properties or relative properties of one or more or all of the spinal rods or spinal posts; b) identifying the location that a plurality of spinal rods or spinal posts are to be used in the spinal surgical procedure; c) determining the desired flexibility or bendability of each spinal rod or post that is be used in a certain location of the spinal during the spinal surgical procedure; d) selecting a spinal rod or post that has the desired flexibility or bendability (and wherein such selection is optionally based on a marking on the spinal rod or spinal post; e) selecting the spinal bone screws (e.g., pedicle screws) for use in the spinal procedure, and wherein each of the spinal bone screws include a top portion that includes a post opening that is configured to receive a portions of the spinal rod or spinal post so that the spinal rod or spinal post can be secured in the post opening to thereby secure the spinal rod or spinal post to the top portion of the spinal bone screw, and wherein the shape, size and configuration of the posting poring for a plurality of all of the spinal bone screws is the same; f) inserting two or more spinal bone screws into the bone (e.g., spine, etc.) of a patient; and g) securing the selected spinal rod or spinal post that have the desired flexibility or bendability to the spinal bone screws during the spinal surgical procedure.

Another and/or alternative non-limiting object of the present disclosure is the provision of a method of providing spinal fusion components to a medical provider or healthcare system comprising; a) providing a plurality of spinal screws or pedicle screws, each of the spinal screws includes a body portion that is configured to be inserted into the spine and a head portion that is connected to the body portion and which head portion is configured to include a post opening and a securing arrangement, wherein the longitudinal length of the body portion is 20-80 mm (and all values and ranges therebetween), wherein the post opening is designed to allow a portion of a spinal rod or post to be inserted there through, wherein the configuration of the post opening is the same of the spinal screw irrespective of the longitudinal length of the body, and the securing arrangement is designed to secure a spinal rod or spinal post in the post opening; b) provided a plurality of spinal rods or spinal posts that i) are formed of the same metal alloy (e.g., refractory metal alloy [e.g., MoRe alloy, Re alloy, Mo alloy, Ta alloy, W alloy, etc.], metal alloy that includes at least 15 awt. % rhenium, etc.), ii) have a body member that is generally cylindrical in shape and has a constant cross-sectional shape and cross-sectional size o diameter along 60-100% (and all values and ranges therebetween) of the longitudinal length of the body member, iii) the body member of a plurality of the spinal rods or spinal posts has different physical properties with regard to at least the flexibility or bendability, yield strength and/or ultimate tensile strength due at least in part to the subjecting of the metal alloy on the body member of the plurality of the spinal rods or spinal posts to different final heating processes, and iv) the spinal rod or spinal posts optionally have one or more markings (e.g., color coatings, etchings on outer surface, symbols on outer surface, etc.) that are used to visually indicate the properties or relative properties of one or more or all of the spinal rods or spinal posts; c) enabling the medical provider or healthcare system to use a plurality of provided spinal screws for use in a particular medical procedure; d) enabling the medical provider or healthcare system to use one or more of the provided spinal rods or posts for use in the particular medical procedure, and wherein the obtained spinal rod or spinal post will properly fit in and be secured to the opening of the spinal screw.

Another and/or alternative non-limiting object of the present disclosure is the provision of medical devices that are at least partially formed of a refractory metal alloy or a metal alloy that includes at least 15 awt. % rhenium that overcome several unmet needs that exist in comparable medical device formed of CoCr alloys, TiAlV alloys, and stainless steel, namely one or more of 1) not having to form a large hole in large arterial vessels or other blood vessels for initial insertion of the crimped medical device into the atrial vessel or other blood vessel, thereby reducing the incidence of lethal bleeding during a treatment; 2) enabling the medical device to be delivered and implanted in abnormally shaped heart valves or through an abnormally shaped arterial vessel due to calcination in the medical device and/or calcination and/or plaque in the arterial vessel by creating a medical device (e.g., stent, medical device, etc.) having a reduced crimped profile that is smaller than medical devices formed of CoCr alloys, TiAlV alloys, and stainless steel; 3) reducing the incidence of a perivalvular leak and/or other types of leakage about the implanted medical device when the medical device is expanded in the treatment region by using a frame formed of the refractory metal alloy or the metal alloy that includes at least 15 awt. % rhenium that better conforms to the shape of the abnormally shaped heart valve orifice upon expansion of the medical device comparted to prior art medical devices formed of CoCr alloys, TiAlV alloys, and stainless steel, thereby reducing the incidence of stroke and/or by increasing the incidence of success of the implanted medical device; 4) improving the radial strength of the expanded struts, posts, and/or strut joints in the expandable frame and the strength of the expandable frame itself after expansion the medical device; 5) reducing the amount of recoil of the expandable frame during the crimping and/or expansion of the expandable frame of the medical device; 6) enabling the medical device to be used in a heart that has a permanent pacemaker; 7) reducing the incidence of minor stroke during the insertion and operation of the medical device at the treatment area; 8) reducing the incidence of coronary ostium compromise; 9) improving foreshortening; 10) reducing further aortic valve calcification and/or calcification in a blood vessel after implantation of the medical device; 11) reducing the need for multiple crimping cycles when inserting the medical device on a catheter or other type of delivery system; 12) reducing the incidence of frame/stent fracture during the crimping and/or expansion of the medical device; 13) reducing the incidence of biofilm-endocarditis after implantation of the medical device; 14) reducing allergic reactions to the medical device after implantation of the medical device; 15) improving the hydrophilicity of the medical device to improve tissue growth on and/or about the implanted medical device, 16) reduce the magnetic susceptibility of the medical device, 17) reduce the toxicity of the medical device, 18) reduce the amount of metal ion release from the medical device, and/or 19) increasing the longevity of leaflets and/or stent/frame and/or other components of the medical device after insertion of the medical device.

These and other advantages will become apparent to those skilled in the art upon the reading and following of this description.

### BRIEF DESCRIPTION OF THE DRAWINGS

Reference may now be made to the drawings, which illustrate various non-limiting embodiments that the disclosure may take in physical form and in certain parts and arrangement of parts wherein:

FIG. 1 illustration a non-limiting process for the final heating of a medical device.

FIG. 2 is a graph that illustrates the amount of recoil of several different metal alloys.

FIG. 3 is an illustration that compares the conformability of a metal strip or wire formed of the refractory metal alloy or the metal alloy that includes at least 15 awt. % rhenium to the shape of a die surface as compared to the conformity of a metal strip or wire of CoCr alloy on the same die surface.

FIGS. 4A-4C illustrate stress vs. reduction in percent area graphs of TiAlV alloy, CoCr alloy and MoRe alloy.

FIG. 5 is a graph that illustrates the differences of stiffness and yield strength of a MoRe alloy, CoCr alloy and TiAlV alloy.

FIGS. 6-8 are graphs that illustrate the strength and fatigue ductility of a TiAlV alloy, CoCr alloy and MoRe alloy.

FIG. 9 illustrates the hydrophilicity of a MoRe alloy, a CoCr alloy, and a TiAlV alloy.

FIGS. 10-11 illustrate the ion release rates of a refractory metal alloy or a metal alloy that includes at least 15 awt. % rhenium such as MoRe.

FIG. 12 illustrates the ion release rates in tissue from a MoRe alloy, a CoCr alloy, and a TiAlV alloy.

FIG. 13 is a Table that illustrated different final heat treat spinal rods and the force that was required to bend each of the spinal rods.

Although specific terms are used in the following description for the sake of clarity, these terms are intended to refer only to the particular structure of the embodiments selected for illustration in the drawings and are not intended to define or limit the scope of the disclosure. In the drawings and the following description below, it is to be understood that like numeric designations refer to components of like function.

The singular forms “a,” “an,” and “the” include plural referents unless the context clearly dictates otherwise.

As used in the specification and in the claims, the term “comprising” may include the embodiments “consisting of” and “consisting essentially of” The terms “comprise(s),” “include(s),” “having,” “has,” “can,” “contain(s),” and variants thereof, as used herein, are intended to be open-ended transitional phrases, terms, or words that require the presence of the named ingredients/steps and permit the presence of other ingredients/steps. However, such description should be construed as also describing compositions or processes as “consisting of” and “consisting essentially of” the enumerated ingredients/steps, which allows the presence of only the named ingredients/steps, along with any unavoidable impurities that might result therefrom, and excludes other ingredients/steps.

Numerical values in the specification and claims of this application should be understood to include numerical values which are the same when reduced to the same number of significant figures and numerical values which differ from the stated value by less than the experimental error of conventional measurement technique of the type described in the present application to determine the value.

All ranges disclosed herein are inclusive of the recited endpoint and independently combinable (for example, the range of “from 2 grams to 10 grams” is inclusive of the endpoints, 2 grams and 10 grams, and all the intermediate values).

The terms “about” and “approximately” can be used to include any numerical value that can vary without changing the basic function of that value. When used with a range, “about” and “approximately” also disclose the range defined by the absolute values of the two endpoints, e.g., “about 2 to about 4” also discloses the range “from 2 to 4.” Generally, the terms “about” and “approximately” may refer to plus or minus 10% of the indicated number.

Percentages of elements should be assumed to be percent by weight of the stated element, unless expressly stated otherwise.

The medical device in accordance with the present disclosure can be any medical device that can be inserted or otherwise applied to a patient. Non-limiting medical devices in accordance with the present disclosure include orthopedic devices, PFO devices, stents, valves (e.g., heart valve, etc.), spinal implants, devices for treating aneurysms, occlusive devices for use in blood vessels and other body passageways, flow adjusting and/or diversion devices for blood vessels, devices for de-endothelializing a wall of an aneurysm, frame and other structures for use with a spinal implants, vascular implant, graft, dental implant, wire for used in medical procedures, bone implant; artificial disk, artificial spinal disk, prosthetic implant or device to repair, replace and/or support a bone and/or cartilage, bone plate, nail; rod, screw, post; cage, plate, pedicle screw, joint system, anchor, bone spacer, or disk that is used in a body to support a structure, mount a structure, and/or repair a structure in a body such as, but not limited to, a human body, animal body, etc.

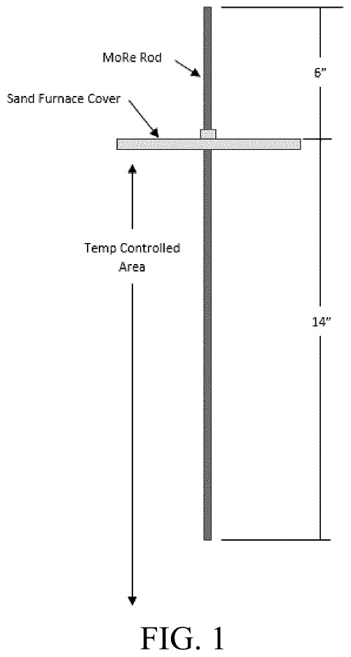

As discussed above, FIG. 1 illustrates a non-limiting process for subjecting a portion or all of a medical device to a final heating process so as to reduce the yield strength and/or ultimate tensile strength of the portion of the refractory metal alloy or the metal alloy that includes at least 15 awt. % rhenium that has been subjected to the final heating process. As illustrated in FIG. 1, the rod or post that is being subjected to the final heating is illustrated as being 20 inches; however, it will be appreciated that other rod or post lengths can be used. As also illustrated in FIG. 1, the bottom portion of the rod or post that is located in the heating oven is about 14 inches and the top portion of the rod or post located outside of the heating oven is about 6 inches; however, it can be appreciated that more of the rod or post or less of the rod or post can be positioned in the heating oven for a final heating process. In one non-limiting arrangement, the rod portion that is located in the heating oven is exposed to temperatures of up to 1000° C. (e.g., 100-1000° C. and all values and ranges therebetween). In one particular non-limiting arrangement, the rod portion that was located in the furnace was initially exposed to a temperature of 10-250° C. (and all values and ranges therebetween, 90-160° C.) (e.g., Preheat step), and then the heat was increased to 500-1000° C. (and all values and ranges therebetween, 550-700° C.) over a period of 0.5-10 hours (and all values and ranges therebetween, 1-3 hours), and thereafter the heat in the heating oven was maintained at the maximum temperature for 1-15 hours (and all values and ranges therebetween, 2-10 hours).

Once the final heating of the rod or post is completed, the rod or post can be removed from the heating oven and allowed to slowly cool. Generally, the rate of cooling after the final heat treatment step is less than 100° C./s (e.g., less than 1-100° C./s and all values and ranges therebetween), and typically less than 50° C./s, and more typically less than 25° C./s.

Once the rod or post has been cooled, the rod or post can then be cut to desired lengths (e.g., 20-500 mm [and all values and ranges therebetween] with a diameter of 3-8 mm [and all values and ranges therebetween]). As can be appreciated, the cut portions of the rod or post can be a) rod or post portions that were subjected to the final heating process, b) rod or post portions that were not subject to the final heating process, or c) rod or post portions where a portion was subjected to the final heating process and a portion that was not subjected to the final heating process. The physical properties of the rod or post portions can a) be uniform or substantially uniform along the longitudinal length of the rod or post portions, or b) vary along the longitudinal length of the rod or post portions.

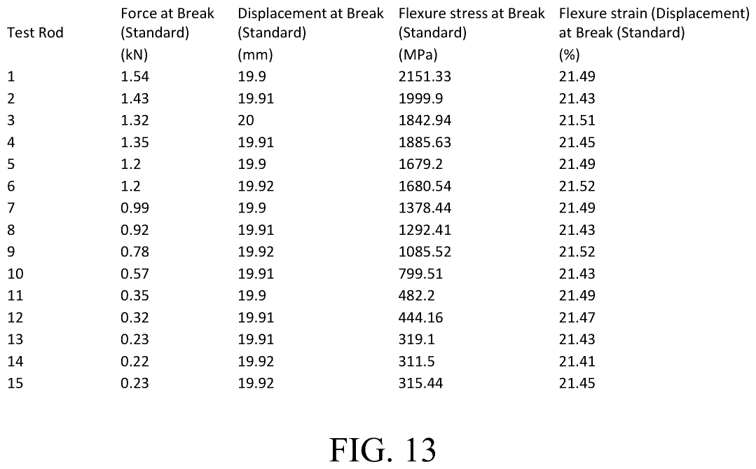

As illustrated in FIG. 13, a refractory metal alloy or the metal alloy that includes at least 15 awt. % rhenium such as a MoRe alloy (e.g., 40-60 wt. % Re & 40-60 wt. % Mo) is subjected to different final heating processes wherein Test Rod 1 was not subjected to a final heating process and Test Rod 14 was subjected to the final heating process for the longest time period. Test Rods 2-13 were incrementally subjected to the final heating process for a longer period of time. As illustrated in Table 1, the longer the rod or post was subjected to the final heating process (which maximum temperature for each of the final heating processes was the same [e.g., 550-650° C., 600° C., etc.], the less force that was required to obtain a bending displacement of about 21.5%. As such, Table 1 illustrates rods or posts having the same cross-sectional shape and same cross-sectional area and formed of the same refractory metal alloy or the metal alloy that includes at least 15 awt. % rhenium can have different yield strength and ultimate tensile strengths after subjecting the rods or posts to different final heating processes. None of the rods or posts represented in FIG. 13 were quenched after the final heating processes. The rods or posts were allowed to cool by exposure of the rods or posts to the ambient temperature (e.g., 65° F.-85° F.) while placed in a non-oxidizing environment.

A non-limiting method for using a plurality of rods or posts having the same or similar size, shape and configuration and that are formed of the same refractory metal alloy or the metal alloy that includes at least 15 awt. % rhenium in a medical procedure, but the rods or posts have different physical properties with regard to at least the flexibility or bendability, yield strength and/or ultimate tensile strength includes: a) provided a plurality of spinal rods or spinal posts that i) are formed of the same metal alloy (e.g., refractory metal alloy [e.g., MoRe alloy, Re alloy, Mo alloy, Ta alloy, W alloy, etc.], metal alloy that includes at least 15 awt. % rhenium, etc.), ii) have a body member that is generally cylindrical in shape and has a constant cross-sectional shape and cross-sectional size or diameter along 60-100% (and all values and ranges therebetween) of the longitudinal length of the body member, iii) the body member of a plurality of the spinal rods or spinal posts has different physical properties with regard to at least the flexibility or bendability, yield strength and/or ultimate tensile strength due at least in part to the subjecting of the metal alloy on the body member of the plurality of the spinal rods or spinal posts to different final heating processes, and iv) the spinal rod or spinal posts optionally have one or more markings (e.g., color coatings, etchings on outer surface, symbols on outer surface, etc.) that are used to visually indicate the properties or relative properties of one or more or all of the spinal rods or spinal posts; b) identifying the location that a plurality of spinal rods or spinal posts are to be used in the spinal surgical procedure; c) determining the desired flexibility or bendability of each spinal rod or post that is be used in a certain location of the spinal during the spinal surgical procedure; d) selecting a spinal rod or post that has the desired flexibility or bendability (and wherein such selection is optionally based on a marking on the spinal rod or spinal post; e) selecting the spinal bone screws (e.g., pedicle screws) for use in the spinal procedure, and wherein each of the spinal bone screws include a top portion that includes a post opening that is configured to receive a portions of the spinal rod or spinal post so that the spinal rod or spinal post can be secured in the post opening to thereby secure the spinal rod or spinal post to the top portion of the spinal bone screw, and wherein the shape, size and configuration of the posting poring for a plurality of all of the spinal bone screws is the same; f) inserting two or more spinal bone screws into the bone (e.g., spine, etc.) of a patient; and g) securing the selected spinal rod or spinal post that have the desired flexibility or bendability to the spinal bone screws during the spinal surgical procedure.

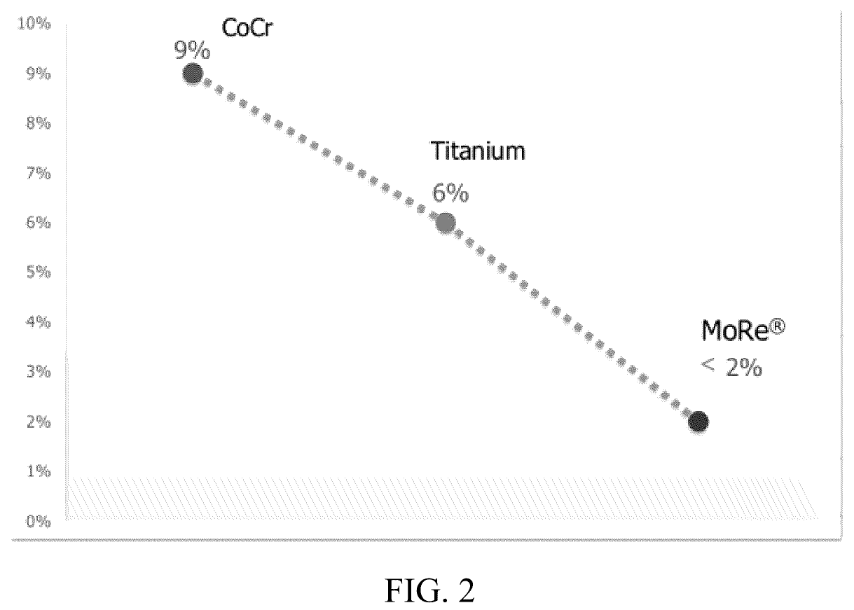

Referring now to FIG. 2, when the medical device includes a frame that can be expanded or crimped (e.g., stent, frame of a heart valve, etc.), and the frame is partially or fully formed of a refractory metal alloy or a metal alloy that includes at least 15 awt. % rhenium, the amount of recoil of the frame after the crimping or expansion of the frame can be less than the amount of recoil of the same sized, shaped and configured frame that is formed of a different metal such as a CoCr alloy or a Ti alloy. As illustrated in FIG. 2, the crimping or expanding of a frame that is formed of CoCr alloy (e.g., 35Co-35Ni-2OCr-10Mo) will recoil by 9% or more (e.g., 9-15% and all values and ranges therebetween) after the radial crimping forces are removed from the frame. Frames formed of Ti alloy (e.g., e.g., Ti-6Al-4V) will recoil by 6% or more (e.g., 6-10% and all values and ranges therebetween) after the radial crimping or expanding forces are removed from the frame. Frames formed of a refractory metal alloy or the metal alloy that includes at least 15 awt. % rhenium (e.g., MoRe alloy, etc.) have a recoil of less than 2% when crimped or expanded as compared to similar shaped, size and configured frames formed of CoCr alloy or Ti alloy. Although not illustrated in FIG. 2, frame that are formed of stainless steel (e.g., 316, 316L) will also recoil by 7% or more (e.g., 6-15% and all values and ranges therebetween) after the radial crimping forces or expansion forces are removed from the frame.

Due to the recoil of frames formed of CoCr alloy, stainless steel or TiAlV alloy, the number of crimping cycles required to crimp a frame formed of refractory metal alloy or the metal alloy that includes at least 15 awt. % rhenium is significantly less than the number of crimping cycles needed to crimp a frame formed of stainless steel, CoCr or TiAlV. Typically, a frame formed of refractory metal alloy or the metal alloy that includes at least 15 awt. % rhenium requires only one crimping cycle to obtain the desired crimped profile of the frame. Typically, a frame formed of stainless steel, CoCr or Ti alloy requires at least two and generally three of more crimping cycles to obtain the desired crimped profile of the frame. Due to such recoil of frames formed of stainless steel, CoCr alloy or Ti alloy, the frame must be repeatedly subjected to a crimping force to attempt to obtain the smallest crimping outer diameter of the crimped frame. The need to subject the frame to multiple crimping cycles or procedures can potentially result in damage to the frame, and/or damage to other components of the medical device (e.g., leaflets, skirts, damage to balloon on the catheter, damage to one or more components on the catheter, etc.). Likewise, a frame formed of a refractory metal alloy or the metal alloy that includes at least 15 awt. % rhenium such as MoRe alloy also has less recoil after being expanded than a frame formed of stainless steel, CoCr or Ti alloy. As such, a frame formed of a refractory metal alloy or the metal alloy that includes at least 15 awt. % rhenium will better conform to the shape of the passageway wherein the frame is expanded. Furthermore, a frame formed of a refractory metal alloy or the metal alloy that includes at least 15 awt. % rhenium will expand to its desired expanded state from a single inflation of the balloon of the balloon delivery catheter. Due to the significant recoil of a frame formed of CoCr alloy, stainless steel (e.g., 316, 316L), and Ti alloy after expansion, the balloon of the balloon delivery catheter typically needs to be inflated multiple times to cause the frame to conform to the shape of the heart passageway wherein the frame is expanded. Such multiple inflations of the balloon can result in increased incidence of damage to a body passageway or the component of the medical device (e.g., leaflets, skirt, etc.).

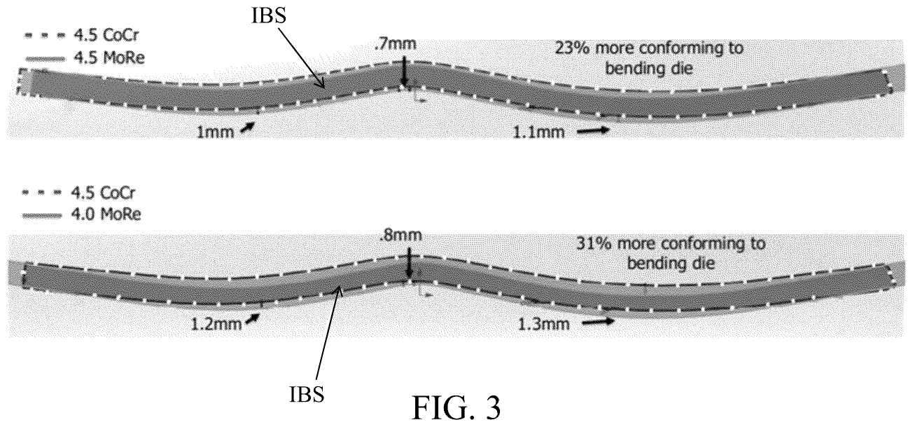

FIG. 3 illustrates increased conformability to bending of a wire, rod or post formed of a refractory metal alloy or a metal alloy that includes at least 15 awt. % rhenium such as MoRe as compared to the same shaped, sized and configured wire, rod or post formed of CoCr alloy. When the frame of a medical device is expanded, the struts and posts of the frame plastically deform (e.g., generally deform outwardly) due to the expansion of the inflatable balloon or from some other expansion device. Generally, the treatment location where the medical device is expanded is not perfectly cylindrical nor has a perfectly shaped circular cross-sectional shape. Generally, the treatment area is damaged and/or includes plaque, calcium deposits and/or other materials (e.g., prior implanted medical devices, etc.) that cause the shape of the treatment area to be non-cylindrical-shaped or have a non-circular cross-sectional shape. As such, frames that can better conform to the irregular shapes in a treatment location result in a medical device that better fits the treatment area. It has been found that frames, wires, rods, struts, posts, etc. that are partially or fully formed of a refractory metal alloy or a metal alloy that includes at least 15 awt. % rhenium (e.g., MoRe alloys, etc.) better conforms to bending and/or shape of the passageway that the medical device is expanded into as compared to the same shaped, sized and configured frames, wires, rods, struts, posts, etc. that are partially or fully formed of metal alloys such as stainless steel, CoCr, Nitinol, and TiAlV alloys and stainless steel (e.g., 316, 316L). FIG. 3 illustrates that when the same sized, shaped and configured MoRe alloy wire and CoCr alloy wire are subjected to the same bending force, the MoRe alloy wire better conforms to the ideal bending shape IBS than the CoCr alloy wire, namely the MoRe alloy wire had 23% to 31% better conformity to the ideal bending shape than the wire formed of CoCr alloy. Tt has been found that wires formed of a refractory metal alloy or a metal alloy that includes at least 15 awt. % rhenium (e.g., MoRe alloy, ReW alloy, MoReCr alloy, MoReTa alloy, MoReTi alloy, WCu alloy, ReCr alloy, Mo alloy, Re alloy, W alloy, Ta alloy, Nb alloy) have about 15-45% (and all values and ranges therebetween) better conformity to bending to an idea bending shape than the same sized, shaped and configured wire formed of stainless steel, CoCr alloy, and TiAlV alloy.

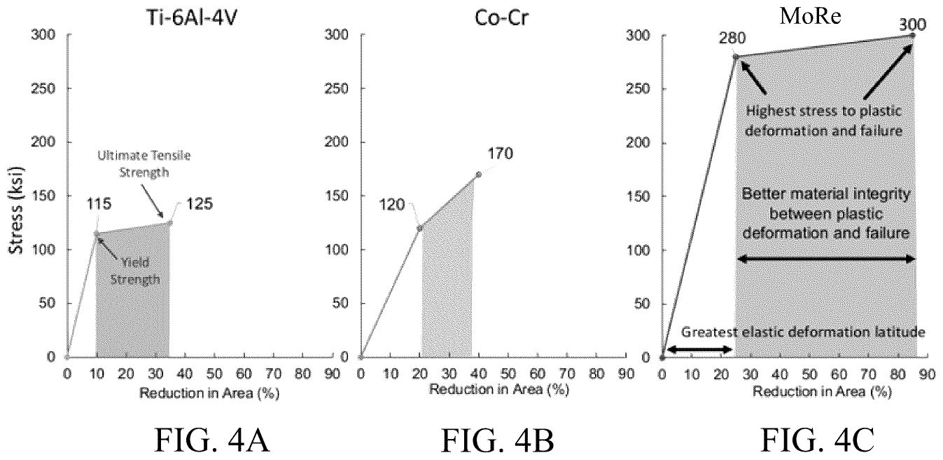

Referring now to FIGS. 4A-4C, there are three graphs that illustrate stress vs. reduction in percent area a wire formed of TiAlV alloy, a CoCr alloy, and a MoRe alloy, and wherein each of the wires has the same size, shape and configuration. These three graphs illustrate that a medical device that is partially or fully formed of a frame, rod, post, strut, etc. made of a refractory metal alloy or a metal alloy that includes at least 15 awt. % rhenium such as MoRe will have has improved properties such as strength, yield strength, ultimate tensile strength, fatigue ductility, greater deformation latitude, material integrity between plastic deformation and failure, and durability as compared to the same shaped, sized and configured medical device that is partially or fully formed of a frame, rod, post, strut, etc. made of materials such CoCr alloys and TiAlV alloys. A medical device that is partially or fully formed of a frame, rod, post, strut, etc. made of refractory metal alloy or the metal alloy that includes at least 15 awt. % rhenium such as MoRe can have a strength of 1.5-5 times or more (and all values and ranges therebetween) greater than medical device that is partially or fully formed of a frame, rod, post, strut, etc. made of CoCr alloys and TiAlV alloys and stainless steel (e.g., 316, 316L).

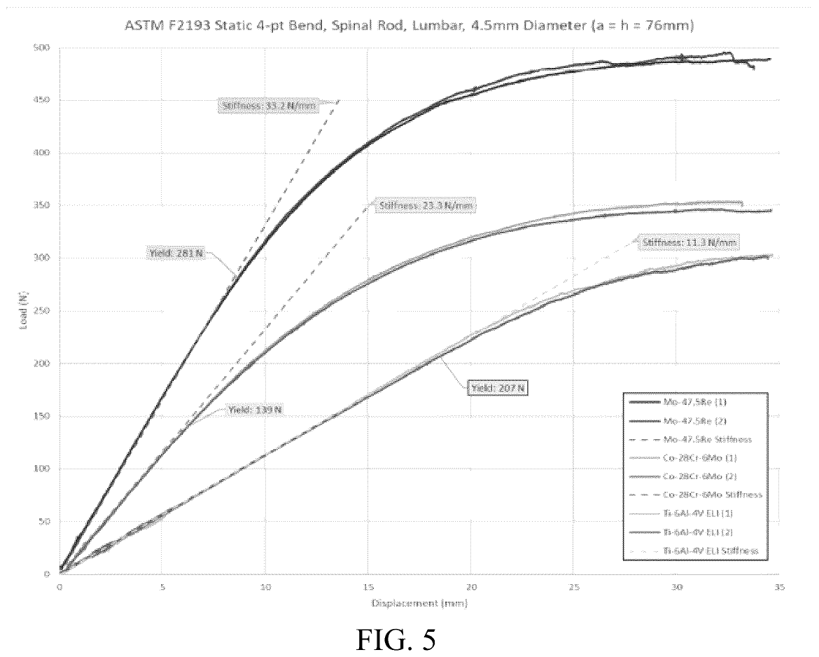

Referring now to FIG. 5, a medical device that is partially or fully formed of a frame, rod, post, strut, etc. made of a refractory metal alloy or a metal alloy that includes at least 15 awt. % rhenium such as MoRe alloy generally has a greater stiffness and yield strength as compared to a medical device that is partially or fully formed of a frame, rod, post, strut, etc. made of CoCr alloys and TiAlV alloys or stainless steel (e.g., 316, 316L). The top curve of FIG. 5 is a MoRe alloy that includes 47.5 wt. % Re and the balance Mo. The middle curve of FIG. 5 is a CoCr alloy that includes 28 wt. % Cr, 6 wt. % Mo and the balance Co. The bottom curve of FIG. 5 is a TiAlV alloy that includes 6 wt. % Al, 4 wt. % V and the balance Ti. Although not illustrated, a refractory metal alloy or a metal alloy that includes at least 15 awt. % rhenium such as MoRe alloy will also have a greater stiffness and yield strength as compared to a medical device that is partially or fully formed of a frame, rod, post, strut, etc. made of stainless steel (e.g., 316, 316L, etc.).

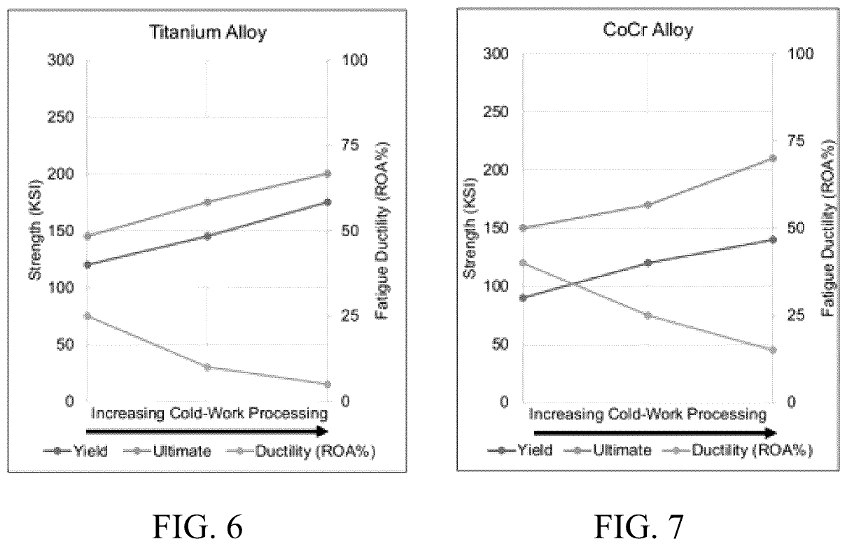

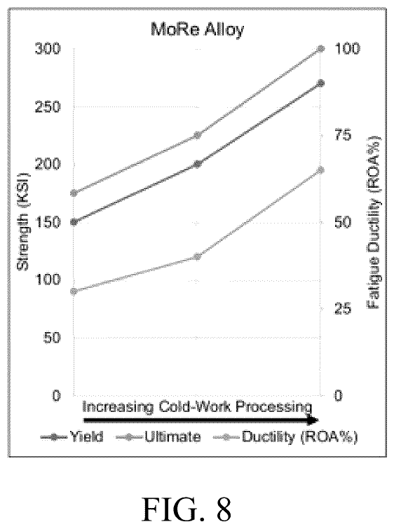

Referring now FIGS. 6-8, there are three graphs that illustrate the yield strength, ultimate strength, and fatigue ductility of a wire formed of TiAlV alloy, CoCr alloy, and MoRe alloy after such alloys are cold worked to reduce the cross-sectional area of the alloy, and wherein the wires have the same size, shape and configuration. After being cold worked, wire that is formed a refractory metal alloy or a metal alloy that includes at least 15 awt. % rhenium such as MoRe alloy generally has greater fatigue ductility, yield strength, and ultimate strength than the shape shaped, sized and configured wire formed of CoCr alloy and TiAlV alloy and stainless steel (e.g., 316, 316L, etc.). Also, the cold working of a wire formed of a refractory metal alloy or a metal alloy that includes at least 15 awt. % rhenium such as MoRe alloy results in increased ductility of the wire. The graphs illustrate the opposite effect on ductility for a wire formed of CoCr alloy and TiAlV alloy and stainless steel (not shown) after the wire is subjected to additional cold working.

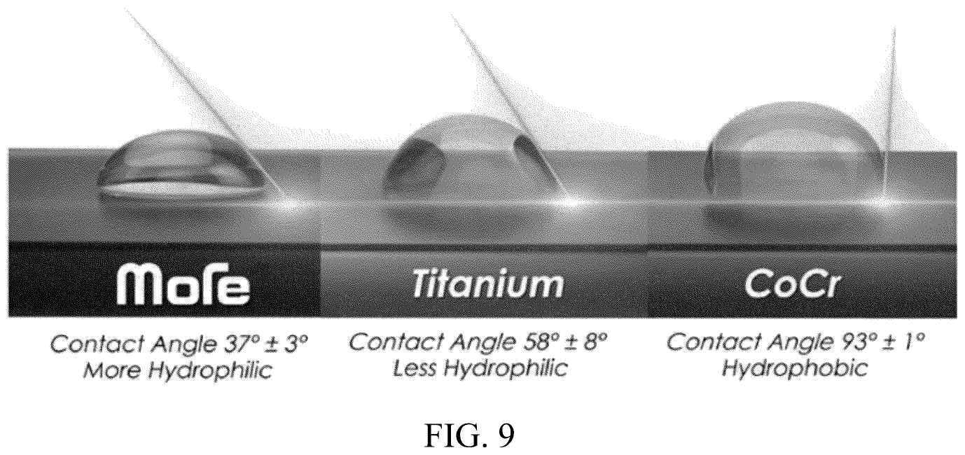

Referring now to FIG. 9, the hydrophilicity of wire formed of a refractory metal alloy or a metal alloy that includes at least 15 awt. % rhenium such as a MoRe alloy is compared to the same shaped, sized and configured wire formed of a CoCr alloy or TiAlV alloy. As illustrated in FIG. 9, CoCr alloys are hydrophobic materials resulting in a large contact angle (93°±1) of a water droplet (e.g., distilled water) positioned on the surface of the wire formed of CoCr alloy. TiAlV alloys are a little more hydrophilic than CoCr alloys and exhibit a contact angle of 58°±8° when a water droplet is positioned on the surface of the wire formed of Ti alloy. Refractory metal alloy or a metal alloy that includes at least 15 awt. % rhenium such as a MoRe alloy has a much greater hydrophilicity than CoCr alloys or TiAlV alloys. The wire formed of MoRe alloy has a contact angle of 37°±3° when a water droplet is positioned on the surface of the wire formed of MoRe alloy. The surface of a frame, wire, strut, post, etc. that is formed of a refractory metal alloy or a metal alloy that includes at least 15 awt. % rhenium generally has a hydrophilicity wherein the contact angle of a water droplet on the surface of such frame, wire, strut, post, etc. is 25°-45° (and all values and ranges therebetween), and typically 30-42°.

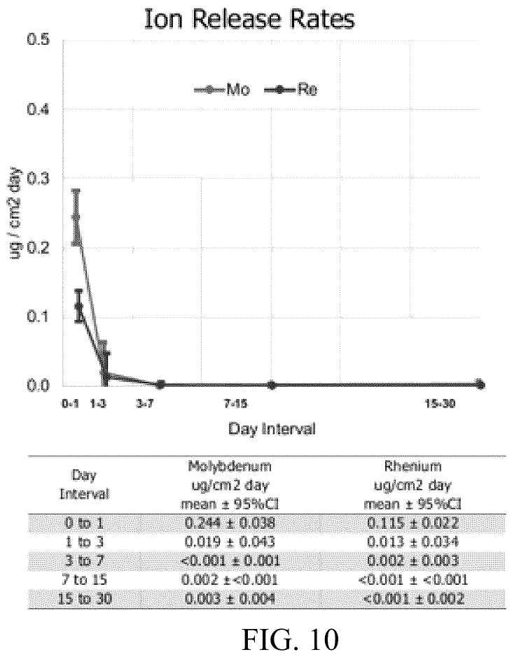

Referring now to FIGS. 10-12, there are illustrated graphs and a table showing the ion release of a refractory metal alloy or a metal alloy that includes at least 15 awt. % rhenium such as MoRe alloy. As illustrated in FIG. 10, during the first day of implanting the medical device that includes a frame, strut, post, wire, etc. form of a refractory metal alloy or a metal alloy that includes at least 15 awt. % rhenium such as a MoRe alloy (e.g., 40-60 wt. % Re and 40-60 wt. % Mo) in a patient, the ion release of molybdenum was about 0.244 μg/cm² per day and the ion release of rhenium was about 0.115 μg/cm² per day. From days 1-3, the ion release of molybdenum was about 0.019 μg/cm² per day and the ion release of rhenium was about 0.013 μg/cm² per day. From days 3-7, the ion release of molybdenum was less than 0.001 μg/cm² per day and the ion release of rhenium was about 0.002 μg/cm² per day. From days 7-15, the ion release of molybdenum was about 0.002 μg/cm² per day and the ion release of rhenium was less than 0.001 μg/cm² per day. From days 15-30, the ion release of Mo was about 0.003 μg/cm² per day and the ion release of rhenium was less than 0.001 μg/cm² per day. The graph illustrates that after the seventh day of implantation in tissue, the ion release of molybdenum and rhenium from the MoRe alloy was effectively nonexistent.

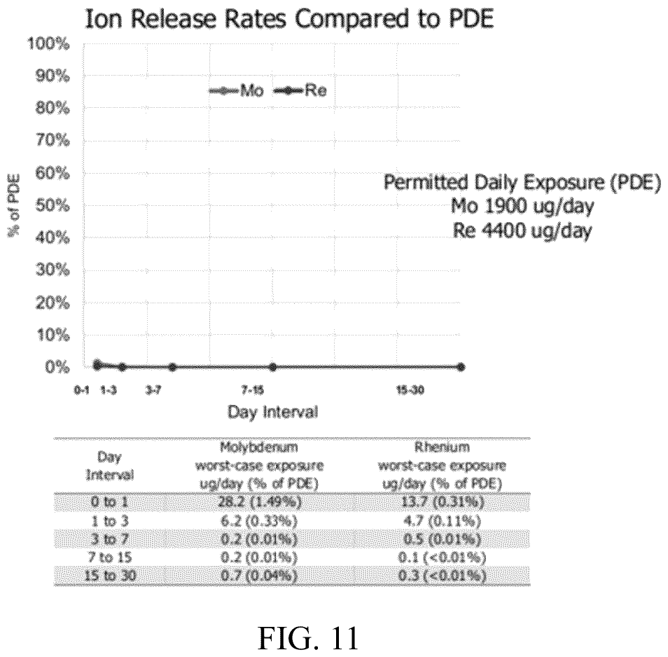

Referring now to FIG. 11, the graph illustrates that the ion release of molybdenum from the MoRe alloy (e.g., 40-60 wt. % Re and 40-60 wt. % Mo) in a frame, strut, post, wire, etc. that was implanted or otherwise inserted in a patient was less than 1.5% of the allowed daily exposure to molybdenum during the first day of insertion of the MoRe alloy in a patient, and such daily ion exposure of molybdenum drops to 0.04% of the allowed daily exposure after 15 days.

FIG. 11 also illustrates that the ion release of rhenium from the MoRe alloy in a frame, strut, post, wire, etc. that was implanted or otherwise inserted in a patient was less than 0.31% of the allowed daily exposure to rhenium during the first day of insertion of the MoRe alloy in a patient, and such daily ion exposure of rhenium drops to less than 0.01% of the allowed daily exposure after 15 days.

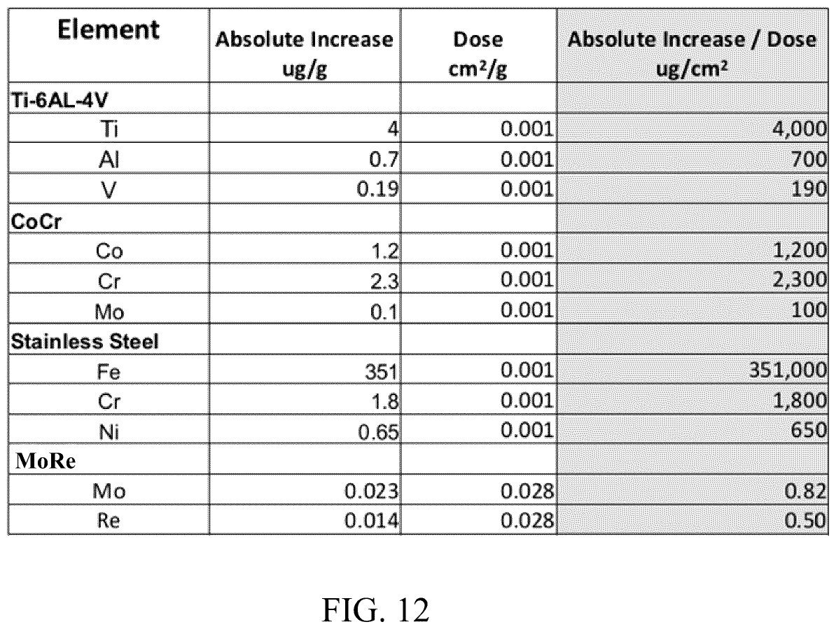

Referring now to FIG. 12, there is illustrated a table showing the amount of primary metals in the alloys of TiAlV, CoCr, stainless steel, and MoRe released into a patient after the medical device including such alloys is inserted into a patient for 90 days. The medical device that includes these allow is the same size, shape and configuration. As illustrated in the table of FIG. 12, the amount of molybdenum and rhenium contained in the tissue surrounding the MoRe alloy (e.g., 40-60 wt. % Re and 40-60 wt. % Mo) after 90 days is significantly lower than any of the primary metals of the other alloys. The amount of molybdenum metal ion in a gram of tissue from a 0.028 cm²/g dose of MoRe (e.g., 40-60 wt. % Re and 40-60 wt. % Mo) in the tissue after 90 days is 0.023 μg/g. As such, the absolute increase in molybdenum metal ion relative to the dose size of the MoRe alloy in the tissue was 0.82. The amount of rhenium metal ion in a gram of tissue from a 0.028 cm²/g dose of MoRe in the tissue after 90 days is 0.014 μg/g. As such, the absolute increase in Re metal ion relative to the dose size of the MoRe alloy in the tissue was 0.5. Based on absolute increase in metal ions in the tissue relative to the dose of the metal alloy in the tissue, both the molybdenum and rhenium content in the tissue from the MoRe alloy after the MoRe alloy was implanted in the tissue for 90 days was over 120 times less than the cobalt or chromium from the CoCr alloy, and many more times less than the other primary metals of the other tested alloys. Generally, the absolute ion release of the primary elements of the refractory metal alloy or the metal alloy that includes at least 15 awt. % rhenium (e.g., primary is an element in an alloy that is at least 2 wt. % of the alloy) relative to the dose of the refractory metal alloy or the metal alloy that includes at least 15 awt. % rhenium in the tissue after 90 days is at least 120 times less than any of the primary components of the alloys of TiAlV, CoCr, stainless steel when a similar does of these other alloys are imp[lated or otherwise inserted in a patient.

It will thus be seen that the objects set forth above, among those made apparent from the preceding description, are efficiently attained, and since certain changes may be made in the constructions set forth without departing from the spirit and scope of the disclosure, it is intended that all matter contained in the above description and shown in the accompanying drawings shall be interpreted as illustrative and not in a limiting sense. The disclosure has been described with reference to preferred and alternate embodiments. Modifications and alterations will become apparent to those skilled in the art upon reading and understanding the detailed discussion of the disclosure provided herein. This disclosure is intended to include all such modifications and alterations insofar as they come within the scope of the present disclosure. It is also to be understood that the following claims are intended to cover all of the generic and specific features of the disclosure herein described and all statements of the scope of the disclosure, which, as a matter of language, might be said to fall therebetween.

1-32. (canceled)

33. A metal rod or tube that that is formed of a metal alloy and which metal rod or tube fully forms a medical device or forms a portion of a medical device; said metal rod or tube has one or more physical properties that are different along a longitudinal length of said metal rod or tube that are selected from a) a different flexibility or bendability along said longitudinal length of said metal rod or tube due to said metal rod or tube being subjected to different processing parameters at different locations along said longitudinal length of said metal rod or tube, b) a different yield strength along said longitudinal length of said metal rod or tube due to said metal rod or tube being subjected to different processing parameters at different locations along said longitudinal length of said metal rod or tube, c) a different ultimate tensile strength along said longitudinal length of said metal rod or tube due to said metal rod or tube being subjected to different processing parameters at different locations along said longitudinal length of said metal rod or tube, and/or d) a different metal alloy crystalline structure along said longitudinal length of said metal rod or tube due to said metal rod or tube being subjected to different processing parameters at different locations along said longitudinal length of said metal rod or tube; said different processing parameters includes subjecting different portions of said metal rod or tube along said longitudinal length of said metal rod or tube to a different final heat treatment process; said different final heat treatment process includes I) subjecting said metal rod or tube to a different final heat treatment temperature at different locations along said longitudinal length of said metal rod or tube, II) exposing said metal rod or tube to said final heat treatment temperature for different time periods at different locations along said longitudinal length of said metal rod or tube; and/or III) cooling said metal rod or tube after subjecting said metal rod or tube to said final heat treatment temperature at different cooling rates at different locations along said longitudinal length of said metal rod or tube.

34. The metal rod or tube as defined in claim 33, wherein said metal alloy includes at least 15 awt. % (atomic percent) rhenium.

35. The metal rod or tube as defined in claim 33, wherein said metal rod or tube has a constant cross-sectional shape and size along 80%-100% of said longitudinal length of said metal rod or tube.

36. The metal rod or tube as defined in claim 34, wherein said metal rod or tube has a constant cross-sectional shape and size along 80%-100% of said longitudinal length of said metal rod or tube.

37. The metal rod or tube as defined in claim 33, wherein at least a portion of said metal rod or tube has not subjected to a quench process after subjecting said metal rod or tube to said final heat treatment temperature; a maximum temperature of said final heat treatment temperature is 500-1000° C.; said final heat treatment is for 0.5-25 hours.

38. The metal rod or tube as defined in claim 36, wherein at least a portion of said metal rod or tube has not subjected to a quench process after subjecting said metal rod or tube to said final heat treatment temperature; a maximum temperature of said final heat treatment temperature is 500-1000° C.; said final heat treatment is for 0.5-25 hours.

39. The metal rod or tube as defined in claim 33, wherein said metal rod or tube has been cooled a rate of less than 100° C./s after said metal rod or tube has been subjected to said final heat treatment process.

40. The metal rod or tube as defined in claim 38, wherein said metal rod or tube has been cooled a rate of less than 100° C./s after said metal rod or tube has been subjected to said final heat treatment process.

41. The metal rod or tube as defined in claim 33, wherein at least a portion of an outer surface of said metal rod or tube includes a marking to indicate a relatively degree of flexibility of said metal rod or tube.

42. The metal rod or tube as defined in claim 40, wherein at least a portion of an outer surface of said metal rod or tube includes a marking to indicate a relatively degree of flexibility of said metal rod or tube.

43. The metal rod or tube as defined in claim 33, wherein said metal alloy includes a) stainless steel that includes at least 15 awt. % (atomic percent) rhenium, b) cobalt-chromium alloy that includes at least 15 awt. % rhenium, c) TiNi alloy that includes at least 15 awt. % rhenium, d) TiAlV alloy that includes at least 15 awt. % rhenium, e) Al alloy that includes at least 15 awt. % rhenium, f) Ni alloy that includes at least 15 awt. % rhenium, g) Ti alloy that includes at least 15 awt. % rhenium, h) W alloy that includes at least 15 awt. % rhenium, i) Cu alloy that includes at least 15 awt. % rhenium, j) beryllium-copper alloy that includes at least 15 awt. % rhenium, k) at least 30 wt. % of one or more of niobium, tantalum, titanium, cobalt, chromium, zirconium or tungsten; and further includes at least 15 awt. % rhenium, l) at least 50 wt. % of one or more of niobium, tantalum, titanium, cobalt, chromium, zirconium or tungsten and further incudes 1-40 wt. % of one or more of aluminum, bismuth, calcium, carbon, cerium oxide, copper, gold, hafnium, iridium, iron, lanthanum, lanthanum oxide, lead, magnesium, manganese, nickel, osmium, platinum, rare earth metals, rhodium, ruthenium, silicon, silver, technetium, tin, titanium, tungsten, vanadium, yttrium, yttrium oxide, zinc, and zirconium oxide; and further includes at least 15 awt. % rhenium, m) at least 60 wt. % tungsten, and at least 15 awt. % rhenium, n) at least 60 wt. % tungsten, at least 15 awt. % rhenium, and at least 1 wt. % molybdenum, o) at least 50 wt. % rhenium, at least 20 wt. % chromium, and 0.1-80 wt. % of one or more of aluminum, bismuth, calcium, carbon, cerium oxide, chromium, cobalt, copper, gold, hafnium, iridium, iron, lanthanum, lanthanum oxide, lead, magnesium, manganese, molybdenum, nickel, niobium, osmium, platinum, rare earth metals, rhodium, ruthenium, silicon, silver, tantalum, technetium, tin, titanium, tungsten, vanadium, yttrium, yttrium oxide, zinc, zirconium, and zirconium oxide, p) greater than 50 wt. % titanium, 15-45 wt. % niobium, 1-10 wt. % zirconium, and 1-15 wt. % tantalum, q) greater than 50 wt. % titanium, 15-45 wt. % niobium, and 1-10 wt. %, r) 30-60 wt. % cobalt, 10-30 wt. % chromium, 5-20 wt. % iron, 5-22 wt. % nickel, and 2-12 wt. % molybdenum, s) 40-60 wt. % zirconium, and 40-60 wt. % molybdenum, t) 90-99.5 wt. % niobium, and 0.5-10 wt. % zirconium, or u) 55-75 wt. % niobium, 18-40 wt. % tantalum, 1-7 wt. % tungsten, and 0.5-4 wt. % zirconium.

44. The metal rod or tube as defined in claim 42, wherein said metal alloy includes a) stainless steel that includes at least 15 awt. % (atomic percent) rhenium, b) cobalt-chromium alloy that includes at least 15 awt. % rhenium, c) TiNi alloy that includes at least 15 awt. % rhenium, d) TiAlV alloy that includes at least 15 awt. % rhenium, e) Al alloy that includes at least 15 awt. % rhenium, f) Ni alloy that includes at least 15 awt. % rhenium, g) Ti alloy that includes at least 15 awt. % rhenium, h) W alloy that includes at least 15 awt. % rhenium, i) Cu alloy that includes at least 15 awt. % rhenium, j) beryllium-copper alloy that includes at least 15 awt. % rhenium, k) at least 30 wt. % of one or more of niobium, tantalum, titanium, cobalt, chromium, zirconium or tungsten; and further includes at least 15 awt. % rhenium, l) at least 50 wt. % of one or more of niobium, tantalum, titanium, cobalt, chromium, zirconium or tungsten and further incudes 1-40 wt. % of one or more of aluminum, bismuth, calcium, carbon, cerium oxide, copper, gold, hafnium, iridium, iron, lanthanum, lanthanum oxide, lead, magnesium, manganese, nickel, osmium, platinum, rare earth metals, rhodium, ruthenium, silicon, silver, technetium, tin, titanium, tungsten, vanadium, yttrium, yttrium oxide, zinc, and zirconium oxide; and further includes at least 15 awt. % rhenium, m) at least 60 wt. % tungsten, and at least 15 awt. % rhenium, n) at least 60 wt. % tungsten, at least 15 awt. % rhenium, and at least 1 wt. % molybdenum, o) at least 50 wt. % rhenium, at least 20 wt. % chromium, and 0.1-80 wt. % of one or more of aluminum, bismuth, calcium, carbon, cerium oxide, chromium, cobalt, copper, gold, hafnium, iridium, iron, lanthanum, lanthanum oxide, lead, magnesium, manganese, molybdenum, nickel, niobium, osmium, platinum, rare earth metals, rhodium, ruthenium, silicon, silver, tantalum, technetium, tin, titanium, tungsten, vanadium, yttrium, yttrium oxide, zinc, zirconium, and zirconium oxide, p) greater than 50 wt. % titanium, 15-45 wt. % niobium, 1-10 wt. % zirconium, and 1-15 wt. % tantalum, q) greater than 50 wt. % titanium, 15-45 wt. % niobium, and 1-10 wt. %, r) 30-60 wt. % cobalt, 10-30 wt. % chromium, 5-20 wt. % iron, 5-22 wt. % nickel, and 2-12 wt. % molybdenum, s) 40-60 wt. % zirconium, and 40-60 wt. % molybdenum, t) 90-99.5 wt. % niobium, and 0.5-10 wt. % zirconium, or u) 55-75 wt. % niobium, 18-40 wt. % tantalum, 1-7 wt. % tungsten, and 0.5-4 wt. % zirconium.

45. A set of spinal surgery materials for use in a spinal surgery comprising:
a. first and second support rods; each of said first and second support rods has a same cross-sectional shape and size along a longitudinal length of said first and second support rods; said first support rod has a flexibility, bendability, yield strength and/or ultimate tensile strength that is different from said second support rod due to said first support rod and second support rods being subjected to I) different processing times of a final heat treatment process, II) different final heat treatment temperatures during said final heat treatment process, and/or III) different cooling rates after said first and second support rods have been subjected to said final heat treatment temperature during said final heat treatment process; said first support rod includes a first rod visual marking; said second support rod includes a second rode visual marking; said first and second rod visual markings are different; and
b. first and second bone screws; said first and second bone screws each include a threaded lower body portion and an upper portion that includes a rod securing arrangement; said body portion of each of said first and second bone screws is formed of the same material; said upper portion of each of said first and second bone screws is formed of the same material; said rod securing arrangement in said upper portion of each of said first and second bone screws includes a rod slot or rod opening that is the same shape and size; said rod slot or rod opening on each of said first and second bone screws is configured to receive a portion of one of said first or second support rods; said rod slot or rod opening on each of said first and second bone screws has a same shape and size.

46. The set of spinal surgery materials as defined in claim 45, wherein said body portion of said first bone screw has a different shape, size and/or longitudinal length from said second bone screw; said first bone screw includes a first screw visual marking; said second bone screw includes a second screw visual marking; said first and second screw visual markings are different.

47. The set of spinal surgery materials as defined in claim 45, wherein each of said first and second support rods is formed of a metal alloy; said metal alloy includes a) stainless steel that includes at least 15 awt. % (atomic percent) rhenium, b) cobalt-chromium alloy that includes at least 15 awt. % rhenium, c) TiNi alloy that includes at least 15 awt. % rhenium, d) TiAlV alloy that includes at least 15 awt. % rhenium, e) Al alloy that includes at least 15 awt. % rhenium, f) Ni alloy that includes at least 15 awt. % rhenium, g) Ti alloy that includes at least 15 awt. % rhenium, h) W alloy that includes at least 15 awt. % rhenium, i) Cu alloy that includes at least 15 awt. % rhenium, j) beryllium-copper alloy that includes at least 15 awt. % rhenium, k) at least 30 wt. % of one or more of niobium, tantalum, titanium, cobalt, chromium, zirconium or tungsten; and further includes at least 15 awt. % rhenium, l) at least 50 wt. % of one or more of niobium, tantalum, titanium, cobalt, chromium, zirconium or tungsten and further incudes 1-40 wt. % of one or more of aluminum, bismuth, calcium, carbon, cerium oxide, copper, gold, hafnium, iridium, iron, lanthanum, lanthanum oxide, lead, magnesium, manganese, nickel, osmium, platinum, rare earth metals, rhodium, ruthenium, silicon, silver, technetium, tin, titanium, tungsten, vanadium, yttrium, yttrium oxide, zinc, and zirconium oxide; and further includes at least 15 awt. % rhenium, m) at least 60 wt. % tungsten, and at least 15 awt. % rhenium, n) at least 60 wt. % tungsten, at least 15 awt. % rhenium, and at least 1 wt. % molybdenum, o) at least 50 wt. % rhenium, at least 20 wt. % chromium, and 0.1-80 wt. % of one or more of aluminum, bismuth, calcium, carbon, cerium oxide, chromium, cobalt, copper, gold, hafnium, iridium, iron, lanthanum, lanthanum oxide, lead, magnesium, manganese, molybdenum, nickel, niobium, osmium, platinum, rare earth metals, rhodium, ruthenium, silicon, silver, tantalum, technetium, tin, titanium, tungsten, vanadium, yttrium, yttrium oxide, zinc, zirconium, and zirconium oxide, p) greater than 50 wt. % titanium, 15-45 wt. % niobium, 1-10 wt. % zirconium, and 1-15 wt. % tantalum, q) greater than 50 wt. % titanium, 15-45 wt. % niobium, and 1-10 wt. %, r) 30-60 wt. % cobalt, 10-30 wt. % chromium, 5-20 wt. % iron, 5-22 wt. % nickel, and 2-12 wt. % molybdenum, s) 40-60 wt. % zirconium, and 40-60 wt. % molybdenum, t) 90-99.5 wt. % niobium, and 0.5-10 wt. % zirconium, or u) 55-75 wt. % niobium, 18-40 wt. % tantalum, 1-7 wt. % tungsten, and 0.5-4 wt. % zirconium.

48. A method for using a set of spinal surgery materials for use in a spinal surgery comprising:
a. providing first and second support rods; each of said first and second support rods has a same cross-sectional shape and size along a longitudinal length of said first and second support rods; said first support rod has a flexibility that is different from said second support rod due to said first support rod and said second support rod being subjected to I) different processing times of a final heat treatment process, II) different final heat treatment temperatures during said final heat treatment process, and/or III) different cooling rates after said first and second support rods have been subjected to said final heat treatment temperature during said final heat treatment process; said first support rod includes a first rod visual marking; said second support rod includes a second rode visual marking; said first and second rod visual markings are different;
b. providing first and second bone screws; said first and second bone screws each include a threaded lower body portion and an upper portion that includes a rod securing arrangement; said body portion of each of said first and second bone screws is formed of the same material; said upper portion of each of said first and second bone screws is formed of the same material; said rod securing arrangement in said upper portion of each of said first and second bone screws includes a rod slot or rod opening that is the same shape and size; said rod slot or rod opening on each of said first and second bone screws is configured to receive a portion of one of said first or second support rods; said rod slot or rod opening on each of said first and second bone screws has a same shape and size;
c. inserting said first bone screw in a first bone in a vertebrae of a patient;
d. inserting said second bone screw in a second bone in said vertebrae of the patient;
e. determining a desired flexibility of a support device that is to be connected to said first and second bone screw;
f. selecting either said first or second support rod to be at least partially used as said support device based on said flexibility of said first and second rods and said desired flexibility of said support device; difference in said flexibility of said first and second support rods is visually identifiable by a medical provider based on said first and second rod markings; and
g. securing said selected first or second support rod to said rod securing arrangement on said first and second bone screws.

49. The method as defined in claim 48, wherein said body portion of said first bone screw has a different shape, size and/or longitudinal length from said second bone screw; said first bone screw includes a first screw visual marking; said second bone screw includes a second screw visual marking; said first and second screw visual markings are different; and further including the steps of i) determining a desired shape, size and/or longitudinal length of a screw for insertion into said first bone; ii) determining a desired shape, size and/or longitudinal length of a screw for insertion into said second bone, and iii) selecting either said first or second bone screw to be inserted into said first bone based on said determined desired shape, size and/or longitudinal length of a screw for insertion into said first bone, and thereafter inserting said selected first or second bone screw into said first bone; a difference in said shape, size and/or longitudinal length of said first and second bone screws is visually identifiable by a medical provider based on said first and second screw markings.

50. The method as defined in claim 48, wherein each of said first and second support rods is formed of a metal alloy; said metal alloy includes a) stainless steel that includes at least 15 awt. % (atomic percent) rhenium, b) cobalt-chromium alloy that includes at least 15 awt. % rhenium, c) TiNi alloy that includes at least 15 awt. % rhenium, d) TiAlV alloy that includes at least 15 awt. % rhenium, e) Al alloy that includes at least 15 awt. % rhenium, f) Ni alloy that includes at least 15 awt. % rhenium, g) Ti alloy that includes at least 15 awt. % rhenium, h) W alloy that includes at least 15 awt. % rhenium, i) Cu alloy that includes at least 15 awt. % rhenium, j) beryllium-copper alloy that includes at least 15 awt. % rhenium, k) at least 30 wt. % of one or more of niobium, tantalum, titanium, cobalt, chromium, zirconium or tungsten; and further includes at least 15 awt. % rhenium, l) at least 50 wt. % of one or more of niobium, tantalum, titanium, cobalt, chromium, zirconium or tungsten and further incudes 1-40 wt. % of one or more of aluminum, bismuth, calcium, carbon, cerium oxide, copper, gold, hafnium, iridium, iron, lanthanum, lanthanum oxide, lead, magnesium, manganese, nickel, osmium, platinum, rare earth metals, rhodium, ruthenium, silicon, silver, technetium, tin, titanium, tungsten, vanadium, yttrium, yttrium oxide, zinc, and zirconium oxide; and further includes at least 15 awt. % rhenium, m) at least 60 wt. % tungsten, and at least 15 awt. % rhenium, n) at least 60 wt. % tungsten, at least 15 awt. % rhenium, and at least 1 wt. % molybdenum, o) at least 50 wt. % rhenium, at least 20 wt. % chromium, and 0.1-80 wt. % of one or more of aluminum, bismuth, calcium, carbon, cerium oxide, chromium, cobalt, copper, gold, hafnium, iridium, iron, lanthanum, lanthanum oxide, lead, magnesium, manganese, molybdenum, nickel, niobium, osmium, platinum, rare earth metals, rhodium, ruthenium, silicon, silver, tantalum, technetium, tin, titanium, tungsten, vanadium, yttrium, yttrium oxide, zinc, zirconium, and zirconium oxide, p) greater than 50 wt. % titanium, 15-45 wt. % niobium, 1-10 wt. % zirconium, and 1-15 wt. % tantalum, q) greater than 50 wt. % titanium, 15-45 wt. % niobium, and 1-10 wt. %, r) 30-60 wt. % cobalt, 10-30 wt. % chromium, 5-20 wt. % iron, 5-22 wt. % nickel, and 2-12 wt. % molybdenum, s) 40-60 wt. % zirconium, and 40-60 wt. % molybdenum, t) 90-99.5 wt. % niobium, and 0.5-10 wt. % zirconium, or u) 55-75 wt. % niobium, 18-40 wt. % tantalum, 1-7 wt. % tungsten, and 0.5-4 wt. % zirconium.

51. A method for forming a set of support rods or tubes that can be used in a surgical procedure comprising:
a. forming first and second rods; each of said first and second rods having a same cross-sectional shape and size along a longitudinal length of said first and second rods; each of said first and second rods formed of a metal alloy; said metal alloy used to form said first and second rods is the same;
b. subjecting said first metal rod or tube to a final heat treatment process to change a flexibility of said metal alloy, to change a bendability of said metal alloy, to change a yield strength of said metal alloy and/or to change an ultimate tensile strength of said metal alloy on said first metal rod or tube;
c. subjecting said second metal rod or tube to a final heat treatment process to change a flexibility of said metal alloy, to change a bendability of said metal alloy, to change a yield strength of said metal alloy and/or to change an ultimate tensile strength of said metal alloy in said second metal rod or tube;
d. cooling said first metal rod or tube after said final heat treatment process;
e. cooling said second metal rod or tube after said final heat treatment process;
f. applying a first rod visual marking to said first metal rod or tube; and
g. applying a second rod visual marking to said second metal rod or tube; and
wherein said first and second rod visual markings are different; and
wherein said flexibility, bendability, yield strength and/or ultimate tensile strength of said first metal rod or tube is different from said second metal rod or tube due to said first metal rod or tube and second metal rods or tubes being subjected to a I) different processing times of a final heat treatment process, II) different final heat treatment temperatures during said final heat treatment process, and/or III) different cooling rates after said first and second support rods have been subjected to said final heat treatment temperature during said final heat treatment process.

52. The method as defined in claim 51, wherein at least a portion of one or both of said first and second metal rods or tubes is absent quenching after subjecting said one or both of said first and second metal rods or tubes said final heat treatment temperature; said final heat treatment temperature is at least 100° C.

53. The method as defined in claim 51, wherein said metal alloy of each of said first and second support rods includes a) stainless steel that includes at least 15 awt. % (atomic percent) rhenium, b) cobalt-chromium alloy that includes at least 15 awt. % rhenium, c) TiNi alloy that includes at least 15 awt. % rhenium, d) TiAlV alloy that includes at least 15 awt. % rhenium, e) Al alloy that includes at least 15 awt. % rhenium, f) Ni alloy that includes at least 15 awt. % rhenium, g) Ti alloy that includes at least 15 awt. % rhenium, h) W alloy that includes at least 15 awt. % rhenium, i) Cu alloy that includes at least 15 awt. % rhenium, j) beryllium-copper alloy that includes at least 15 awt. % rhenium, k) at least 30 wt. % of one or more of niobium, tantalum, titanium, cobalt, chromium, zirconium or tungsten; and further includes at least 15 awt. % rhenium, l) at least 50 wt. % of one or more of niobium, tantalum, titanium, cobalt, chromium, zirconium or tungsten and further incudes 1-40 wt. % of one or more of aluminum, bismuth, calcium, carbon, cerium oxide, copper, gold, hafnium, iridium, iron, lanthanum, lanthanum oxide, lead, magnesium, manganese, nickel, osmium, platinum, rare earth metals, rhodium, ruthenium, silicon, silver, technetium, tin, titanium, tungsten, vanadium, yttrium, yttrium oxide, zinc, and zirconium oxide; and further includes at least 15 awt. % rhenium, m) at least 60 wt. % tungsten, and at least 15 awt. % rhenium, n) at least 60 wt. % tungsten, at least 15 awt. % rhenium, and at least 1 wt. % molybdenum, o) at least 50 wt. % rhenium, at least 20 wt. % chromium, and 0.1-80 wt. % of one or more of aluminum, bismuth, calcium, carbon, cerium oxide, chromium, cobalt, copper, gold, hafnium, iridium, iron, lanthanum, lanthanum oxide, lead, magnesium, manganese, molybdenum, nickel, niobium, osmium, platinum, rare earth metals, rhodium, ruthenium, silicon, silver, tantalum, technetium, tin, titanium, tungsten, vanadium, yttrium, yttrium oxide, zinc, zirconium, and zirconium oxide, p) greater than 50 wt. % titanium, 15-45 wt. % niobium, 1-10 wt. % zirconium, and 1-15 wt. % tantalum, q) greater than 50 wt. % titanium, 15-45 wt. % niobium, and 1-10 wt. %, r) 30-60 wt. % cobalt, 10-30 wt. % chromium, 5-20 wt. % iron, 5-22 wt. % nickel, and 2-12 wt. % molybdenum, s) 40-60 wt. % zirconium, and 40-60 wt. % molybdenum, t) 90-99.5 wt. % niobium, and 0.5-10 wt. % zirconium, or u) 55-75 wt. % niobium, 18-40 wt. % tantalum, 1-7 wt. % tungsten, and 0.5-4 wt. % zirconium.

54. The method as defined in claim 51, wherein a maximum temperature of said final heat treatment temperature of one or both of said first and second metal rods or tubes is 500-1000° C.

55. The method as defined in claim 51, wherein one or both of said first and second metal rods or tubes is subjected to said final heat treatment process for about 0.5-25 hours.

56. The method as defined in claim 51, wherein said step of subjecting said first metal rod or tube to said final heat treatment process includes a) initially increasing a temperature about said first metal rod or tube from a minimum temperature to maximum temperature for a first period of time, and b) maintaining said maximum temperature about said first metal rod or tube for a second period of time; said step of subjecting said second metal rod or tube to said final heat treatment process includes a) initially increasing a temperature about said second metal rod or tube from a minimum temperature to maximum temperature for a first period of time, and b) maintaining said maximum temperature about said second metal rod or tube for a second period of time.

57. The method as defined in claim 51, wherein said minimum temperature is 10-250° C. for one or both of said first and second metal rods or tubes; said first period of time is 0.5-10 hours for one or both of said first and second metal rods or tubes; said second period of time is 0.01-15 hours for one or both of said first and second metal rods or tubes; said first period of time and/or said second period of said for said first metal rod or tube is different from said second metal rod or tube; said maximum temperature is 500-1000° C.

58. The method as defined in claim 51, wherein one or both of said first and second metal rods or tubes is cooled during said step of cooling at a rate of less than 100° C./s after one or both of said metal rods or tubes has been subjected to said final heat treatment temperature.

59. The method as defined in claim 58, wherein said step of cooling occurs a) in non-oxidizing gas environment at a temperature of 10-100° C., b) an inert gas environment at a temperature of 10-100° C., or c) an air environment at a temperature of 10-100° C.
